(19) United States
(12) Patent Application Publication
(10) Pub. No.: US 2024/0363245 A1
(43) Pub. Date: Oct. 31, 2024

(54) CANCER DETECTION THROUGH INTEGRATED ANALYSIS OF WHOLE GENOME SEQUENCING

(71) Applicant: Personal Genome Diagnostics, Inc., Baltimore, MD (US)

(72) Inventors: Andrew Georgiadis, Baltimore, MD (US); Mark Sausen, Morrestown, NJ (US); James R. White, Baltimore, MD (US); Samuel V. Angiuoli, Baltimore, MD (US); David Riley, Baltimore, MD (US)

(73) Assignee: Personal Genome Diagnostics, Inc., Baltimore, MD (US)

(21) Appl. No.: 18/638,669

(22) Filed: Apr. 17, 2024

Related U.S. Application Data
(60) Provisional application No. 63/496,643, filed on Apr. 17, 2023.
(60) Provisional application No. 63/501,219, filed on May 10, 2023.

(51) Int. Cl. G16H 50/20; G16B 30/10; G16B 40/20; G16H 10/40; G16H 20/10; G16H 50/30

(52) U.S. Cl. CPC G16H 50/20; G16B 30/10; G16B 40/20; G16H 10/40; G16H 20/10; G16H 50/30

(57) ABSTRACT

The present disclosure pertains to techniques that leverage machine learning models to identify tumor-specific mutations through an integrated analysis of next generation sequencing data. In a particular aspect, a computer-implemented method is provided that includes generating sequence reads from one or more samples collected from the same patient, generating variant call files by analyzing the sequence reads corresponding respectively to the one or more samples, comparing variant call files to generate a list of candidate somatic variants, generating, by a classification machine learning model, scores for each of the candidate somatic variants in the list of candidate somatic variants, where the scores are generated based on a plurality of classifications generated by the classification machine learning model, determining, based on the scores, a ctDNA status for the patient, where the ctDNA status is either positive or negative, and generating a report that provides the ctDNA status for the patient.

### CROSS-REFERENCE TO RELATED APPLICATIONS

The present application claims priority and benefit from U.S. Provisional Application No. 63/496,643, filed Apr. 17, 2023, and U.S. Provisional Application No. 63/501,219, filed May 10, 2023, the entire contents of each of which are incorporated herein by reference for all purposes.

### FIELD

This disclosure relates to cancer detection techniques that leverage machine learning models to identify tumor-specific mutations through an integrated analysis of next generation sequencing data.

### BACKGROUND

Next-generation sequencing (NGS) technologies have revolutionized routine diagnostics for detecting mutations in clinical laboratories around the world due to its massively parallel sequencing capabilities. Whole-genome sequencing (WGS) is a comprehensive NGS method for analyzing entire genomes (sequences all or substantially all of the 3 billion DNA base pairs that make up an entire genome by determining the order of the nucleotides (A, C, G, T)). The goal of WGS is, typically, to look for genetic aberrations (e.g., single nucleotide variants, deletions, insertions, and structural variants). Because the entire genome is being sequenced, changes in the noncoding or intronic regions of the genome can also be determined. WGS has been particularly impactful in the field of oncology for detecting tumor-specific (somatic) mutations and aiding oncologists in diagnostic and therapeutic management decisions for their patients.

In addition to the standard high coverage NGS approaches typically use, low coverage WGS (1× to 10×) and ultra-low coverage WGS (coverage below 1×) have been developed for analysis of low quality/concentrated DNA samples, such as cell-free circulating tumor DNA (ctDNA) in blood or plasma samples. Low-coverage and ultra-low coverage WGS can accurately assess common genetic variations and large sub-chromosomal and whole chromosomal events using approximately 0.4× sequencing coverage on circulating tumor DNA (ctDNA).

Cell free DNA (cfDNA) is DNA that circulates throughout the body of an individual that has been released by cells undergoing apoptosis or necrosis. CfDNA can be isolated from blood, plasma, sputum, saliva, cerebral spinal fluid, surgical drain fluid, urine, cyst fluid etc. CfDNA isolated from a noncancerous individual mostly comprises white blood cell derived DNA; however, individuals with cancer may also have ctDNA. When a tumor grows in a person's body, small fragments of DNA from the tumor may be found circulating in the person's blood. That ctDNA carries information such as mutations and structural alterations specific to the tumor. For several decades, researchers and clinicians have used ctDNA from the bloodstream of cancer patients to facilitate therapy selection, identify drug resistance, and monitor treatment response by detecting oncology signal through measuring genomic instability. For example, one way clinicians will monitor therapy effectiveness and predict cancer recurrence is by detecting and measuring levels of ctDNA before, during, and after surgical and therapeutic treatment. The practice is often referred to by physicians as minimal or molecular residual disease (MRD) surveillance.

Despite these applications, challenges surrounding the reliability of NGS and WGS to detect somatic mutations, particularly those that are sub-clonal or from low purity tumor samples remain. Further, challenges in distinguishing somatic mutations from germline mutations or technical artifacts have led to concerns regarding the overall accuracy of NGS methods.

### BRIEF SUMMARY

In various embodiments, a computer-implemented method is provided that includes: generating sequence reads from a tumor nucleic acid sample, a noncancerous nucleic acid sample, and a non-tissue nucleic acid sample collected from the same patient, wherein the sequence reads are generated using whole genome sequencing (WGS); generating a tumor variant call file, a noncancerous variant call file, and a non-tissue variant call file by analyzing the sequence reads corresponding respectively to the tumor nucleic acid sample, the noncancerous nucleic acid sample, and the non-tissue sample; comparing the tumor variant call file to the noncancerous variant call file to generate a list of somatic variants; comparing the list of somatic variants to the non-tissue variant call file to generate a list of candidate somatic variants; generating, by a classification machine learning model, scores for each of the candidate somatic variants in the list of candidate somatic variants, wherein the scores are generated based on a plurality of classifications generated by the classification machine learning model; determining, based on the scores, a ctDNA status for the patient, wherein the ctDNA status is either positive or negative; and generating a report that provides the ctDNA status for the patient.

In some embodiments, the tumor nucleic acid sample is any bodily tissue or fluid containing nucleic acid that is considered to be cancer positive, wherein the noncancerous sample is any bodily tissue or fluid containing nucleic acid that is considered to be cancer-free, and wherein the non-tissue sample is any bodily fluid containing nucleic acid that is considered to comprise cell free DNA and circulating tumor DNA.

In some embodiments, the tumor nucleic acid sample is cancer positive tissue, wherein the noncancerous nucleic acid sample is white blood cells, and wherein the non-tissue nucleic acid sample is plasma.

In some embodiments, the computer implemented method of claim 3, wherein the non-tissue nucleic acid sample is circulating tumor DNA.

In some embodiments, the noncancerous nucleic acid sample and the non-tissue nucleic acid sample are collected from the same whole blood sample.

In some embodiments, the tumor nucleic acid sample is sequenced to a depth of at least 50×, wherein the noncancerous nucleic acid sample is sequenced to a depth of at least 30×, and wherein the non-tissue nucleic acid sample is sequenced to a depth of at least 20×.

In some embodiments, the tumor nucleic acid sample is sequenced to a depth of 80×, wherein the noncancerous nucleic acid sample is sequenced to a depth of 40×, and wherein the non-tissue nucleic acid sample is sequenced to a depth of 30×.

In some embodiments, the patient is diagnosed with cancer, received surgery to remove one or more tumors, and received a therapeutic treatment post-surgery.

In some embodiments, the therapeutic treatment is adjuvant chemotherapy therapy.

In some embodiments, the patient is diagnosed with colorectal cancer, head and neck cancer, lung cancer, breast cancer, or melanoma.

In some embodiments, the patient is diagnosed with colorectal cancer.

In some embodiments, the tumor nucleic acid sample, the noncancerous samples, and the non-tissue samples are collected (i) pre-surgery, (ii) during surgery, (iii) about 3 days to about 65 days post-surgery and before receiving a therapeutic treatment, (iv) about every 6 months up to 3 years post-surgery and after receiving the therapeutic treatment, or (v) any combination thereof.

In some embodiments, the tumor variant call file and the noncancerous variant call file are filtered using a set of filtering criteria, and wherein the set of filtering criteria include removing: (i) variants annotated as low confidence, (ii) variants annotated as indels, (iii) variants observed in genomic databases, (iv) variants overlapping simple tandem repeat tracks, (v) variants at genomic positions with less than 10× coverage, (vi) variants at genomic positions with an alternate allele count less than 4 in the tumor nucleic acid sample or greater than 1 in the noncancerous nucleic acid sample, (vii) variants with a variant allele frequency less than 0.05, or (viii) any combination thereof.

In some embodiments, the list of candidate somatic variants comprises substitutions, small indels, chromosomal rearrangements, copy number variation, microsatellite instabilities, or any combination thereof.

In some embodiments, the list of candidate somatic variants includes at least 40,000 to at least 70,000 somatic variants.

In some embodiments, each candidate somatic variant on the list of candidate somatic variants has at least 50 corresponding features.

In some embodiments, the features comprise quality metrics output from sequencing, alignment, and variant calling.

In some embodiments, sequencing features comprise quality scores for any given base in the sequence reads, wherein alignment features comprise quality of alignment, quality of reads, strand information, metrics relating to a complexity of a region in the genome, or any combination thereof, and wherein variant calling features comprise variant confidence scores, quality of a base variant, or any combination thereof.

In some embodiments, prior to generating the scores, the classification model filters, using a set of noncancerous donor samples, the list of candidate somatic variants to generate a filtered list of candidate somatic variants.

In some embodiments, the classification machine learning model is a random forest classifier comprising an ensemble of trees having at least 500 decision trees, wherein: each of the trees generates a score for an input candidate somatic variant, the random forest classifier averages the scores generated by each of the trees to determine a final score, the final score is compared to a predetermined threshold to determine whether a ctDNA status of the non-tissue nucleic acid sample is positive or negative, the ensemble of trees considers at least 50 features associated with the candidate somatic variants, and each tree considers a different subset of features from the at least 50 features to make a prediction for the class.

In some embodiments, the predetermined threshold is a maximum normalized score plus one standard deviation of a cohort of reference variants.

In some embodiments, the final score is greater than or equal to the predetermined threshold and the ctDNA status is positive, and wherein the final score is less than the predetermined threshold and the ctDNA status is negative.

In some embodiments, the ctDNA status is determined by normalizing the scores and comparing the normalized score to a maximum normalized score plus one standard deviation, and wherein the ctDNA status is positive when the normalized score is greater than or equal to the maximum normalized score.

In some embodiments, the ctDNA status represents a post-surgery ctDNA status.

In some embodiments, the ctDNA status is correlated with clinicopathological risk factors to predict survival rate, wherein the clinicopathological risk factors predict recurrence risk, and wherein the clinicopathological risk factors include depth of tumor invasion and spread of tumor to neighboring lymph nodes.

In some embodiments, the correlation between the ctDNA status and the clinicopathological risk factors is included in the report, and wherein the report further describes a recurrence risk and a predicted survival rate of the patient, based on the ctDNA status and clinicopathological risk factors of the patient.

In various embodiments, a computer-implemented method is provided that includes: generating sequence reads from a non-tissue nucleic acid sample collected from a patient, wherein the sequence reads are generated using whole genome sequencing (WGS); generating a non-tissue variant call file by analyzing the sequence reads corresponding to the non-tissue sample; comparing a list of somatic variants to the non-tissue variant call file to generate a list of candidate somatic variants; generating, by a classification machine learning model, scores for each of the candidate somatic variants in the list of candidate somatic variants, wherein the scores are generated based on a plurality of classifications generated by the classification machine learning model; determining, based on the scores, a ctDNA status for the patient, wherein the ctDNA status is either positive or negative; and generating a report that provides the ctDNA status for the patient.

In various embodiments, a computer-implemented method is provided that includes: accessing a labeled training dataset, wherein the labeled training dataset comprises ground truth true positive variants and associated features collected from patients with cancer and ground truth false positive variants and associated features collected from noncancerous patients; training, a classification model, using the labeled training dataset to generate scores, wherein the training is an iterative process starting at a first node of a first tree that comprises: inputting a portion of the labeled training data into the classification model, selecting, at random, a number of variant features from the portion of the labeled training dataset, determining which of the variant features from the number of variant features that provides a best binary split, wherein the determination is based on a subset of variant features that minimizes an objective function, and assigning, to the first node, the determined variant feature; repeating the iterative process at a second and subsequent nodes of the classification model for a number of iteration or epochs; repeating the iterative process at a first node of a second and subsequent tree until all variant features have been assigned to a tree; and outputting a trained classification model.

In some embodiments, a system is provided that includes one or more processors, and a memory that is coupled to the one or more processors and stores a plurality of instructions which, when executed by the one or more processors, cause the one or more processors to perform any of the methods disclosed herein.

In some embodiments, a computer-program product is provided that is tangibly embodied in a non-transitory computer-readable memory that includes instructions which, when executed by the one or more processors, cause the one or more processors to perform any of the methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The drawings illustrate certain embodiments of the technology and are not limiting. For clarity and ease of illustration, the drawings are not made to scale, and in some instances, various aspects may be shown exaggerated or enlarged to facilitate an understanding of particular embodiments.

FIG. 1 shows statistical data associated with post-surgery ACT treatments for stage III colon cancer patient outcomes.

FIG. 2 shows a computing environment in accordance with various embodiments.

FIG. 3 shows an exemplary sample processing and bioinformatic workflow for detecting ctDNA in a non-tissue sample in accordance with various embodiments.

FIG. 4 shows a block diagram of an exemplary machine learning pipeline comprising several subsystems that work together to train, validate, and implement one or more machine learning models in accordance with various embodiments.

FIGS. 5A-5B show exemplary workflows for using a machine learning pipeline during inference phase (FIG. 5A) and the training of a classification model (FIG. 5B) in accordance with various embodiments.

FIG. 6 shows an exemplary illustration of a random forest machine learning model in accordance with various embodiments.

FIG. 7 shows an example of a computing environment to perform the disclosed techniques in accordance with various embodiments.

FIG. 8 shows an overview of the PROVENC3 study. FIG. 8A illustrates the PROVENC3 study population and main exclusion criteria from final analysis. FIG. 8B an exemplary schematic of the PROVENC3 study design showing the number of patients analyzed for each research question in accordance with various embodiments.

FIG. 9 shows a dot graph (FIG. 9A) and a box and whiskers (FIG. 9B) for post-surgery ctDNA status and cfDNA concentration in accordance with various embodiments.

FIG. 10A shows an exemplary schematic of tumor-informed detection of ctDNA through integrated WGS analysis and machine learning model techniques in accordance with various embodiments.

FIG. 10B illustrates the analytical sensitivity studies performed using contrived reference models derived from commercially available cell lines in accordance with various embodiments.

FIG. 10C illustrates the analytical specificity studies performed in accordance with various embodiments.

FIG. 10D demonstrates the high reproducibility of the external contrived reference control sample (SeraSeq gDNA TMB-mix Score 26) for the estimated tumor fraction across 45 independent runs evaluated for the PROVENC3 clinical study (n=45 runs, CV=7.2%)

FIG. 11A is a graph showing the analytical specificity of across noncancerous donor plasma samples. FIG. 11B is a graph illustrating the analytical sensitivity of the methods in accordance with various embodiments.

FIG. 12 shows the results of an assessment of ctDNA status across multiple solid tumor types in accordance with various embodiments.

FIGS. 13A-13E shows a detection of ctDNA post-surgery is independently associated with recurrence at 3-years in ACT treated stage III colon cancer in accordance with various embodiments. FIG. 13A shows Kaplan-Meier estimate for TTR stratified by post-surgery ctDNA status and FIG. 13B shows the proportion of patients at risk of recurrence after three years. FIG. 13C shows Kaplan-Meier estimate for TTR stratified by clinicopathological risk and FIG. 13D shows the proportion of patients at risk of recurrence after three years. FIG. 13E shows Kaplan-Meier estimate for TTR stratified by clinicopathological risk and ctDNA status and FIG. 13F shows the proportion of patients at risk of recurrence after three years. Abbreviations: ctDNA, circulating tumor DNA; ACT, adjuvant chemotherapy; TTR, time to recurrence; ctDNA-pos, ctDNA positive; ctDNA-neg, ctDNA-negative; n, number; Clin.path., Clinicopathological; HR, Hazard ratio.

FIG. 14 shows Kaplan-Meier estimates for cox regression analyses for clinicopathological low-risk (FIG. 14A) and high-risk (FIG. 14B) groups stratified by post-surgery ctDNA status, including confidence intervals, in accordance with various embodiments.

FIG. 15 shows time to recurrence based on ctDNA status in accordance with various embodiments. 15A shows a Kaplan-Meier estimate for TTR stratified by post-surgery ctDNA status for all patients experiencing disease recurrence. FIG. 15B is a swimmer plot for all patients with a recurrence (n=47). FIG. 15C is a Kaplan-Meier estimate for TTR stratified by post-ACT ctDNA status. FIG. 15D shows the proportion of patients at risk of recurrence after three years. Abbreviations: TTR, time to recurrence; ctDNA, circulating tumor DNA; ACT, adjuvant chemotherapy; HR, Hazard ratio.

### TERMS

As used herein, the singular forms “a,” “an,” and “the” include plural references unless the context clearly dictates otherwise. For example, references to “the method” include one or more methods, and/or steps of the type described herein, which will become apparent to those persons skilled in the art upon reading this disclosure and so forth. Additionally, the term “a nucleic acid” includes a plurality of nucleic acids, including mixtures thereof.

The terms “about” and “approximately” are used interchangeably and mean within an acceptable error range for the particular value as determined by one of ordinary skill in the art, and thus depend in part on how the value is measured or determined, e.g., the limitations of the measurement system. For example, the term “substantially,” “approximately,” or “about” may be substituted with “within [a percentage] of” what is specified, where the percentage includes 0.1, 1, 5, and 10 percent. Where particular values are described in the application and claims, unless otherwise stated, the term “about” means within an acceptable error range for the particular value.

As used herein, the term “allele” refers to any alternative forms of a gene at a particular locus. There may be one or more alternative forms, all of which may relate to one trait or characteristic at the specific locus. In a diploid cell of an organism, alleles of a given gene can be located at a specific location, or locus (loci plural) on a chromosome. The genetic sequences that differ between different alleles at each locus are termed “variants,” “polymorphisms,” or “mutations.” The term “single nucleotide polymorphisms (SNP)” is used interchangeably with “single nucleotide variants (SNVs)” throughout.

The terms “allele frequency” or “allelic frequency,” as used herein, generally refer to the relative frequency of an allele (e.g., variant of a gene) in a sample, e.g., expressed as a fraction or percentage. In some cases, allelic frequency may refer to the relative frequency of an allele (e.g., variant of a gene) in a sample, such as a CFNA sample. In some cases, allelic frequency may refer to the relative frequency of an allele (e.g., variant of a gene) in a sample, such as a CFNA standard. The allelic frequency of a mutant allele may refer to the frequency of the mutant allele relative to the wild-type allele in a sample, e.g., a cell-free nucleic acid sample. For example, if a sample includes 100 copies of a gene, five of which are a mutant allele and 95 of which are the wild-type allele, an allelic frequency of the mutant allele is about 5/100 or about 5%. A sample having no copies of a mutant allele (e.g., about 0% allelic frequency) may be used, for example, as a negative control. A negative control may be a sample in which no mutant allele is expected to be detected. A sample including a mutant allele at about 50% allelic frequency may, for example, be representative of a germline heterozygous mutation.

Cancer refers to an abnormal state or condition characterized by rapidly proliferating cell growth. Rapidly proliferating cells may be categorized as pathologic (i.e., characterizing or constituting a disease state), or may be categorized as non-pathologic (i.e., a deviation from normal but not associated with a disease state). In general, cancer will be associated with the presence of one or more tumors (i.e., abnormal cell masses). In addition, cancer cells can spread locally or through the bloodstream and lymphatic system to other parts of the body. Examples of cancer include malignancies of various organ systems, such as lung cancers, breast cancers, thyroid cancers, lymphoid cancers, gastrointestinal cancers, and -urinary tract cancers. Cancer can also refer to adenocarcinomas, which include malignancies such as colon cancers, renal-cell carcinoma, prostate cancer and/or testicular tumors, non-small cell carcinoma of the lung, cancer of the small intestine, and cancer of the esophagus. Carcinomas are malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures. A “sarcoma” refers to a malignant tumor of mesenchymal derivation. “Melanoma” refers to a tumor arising from a melanocyte. Melanomas occur most commonly in the skin and are frequently observed to metastasize widely.

The term “cell-free nucleic acid” or “CFNA” refers to extracellular nucleic acids, as well as circulating free nucleic acid. As such, the terms “extracellular nucleic acid,” “cell-free nucleic acid” and “circulating free nucleic acid” are used interchangeably. Extracellular nucleic acids can be found in biological sources such as blood, urine, and stool. CFNA may refer to cell-free DNA (cfDNA), circulating free DNA (cfDNA), cell-free RNA (cfRNA), or circulating free RNA (cfRNA). CFNA may result from the shedding of nucleic acids from cells undergoing apoptosis or necrosis. Previous studies have demonstrated that CFNA, for example cfDNA, exists at steady-state levels and can increase with cellular injury or necrosis. In some cases, CFNA is shed from abnormal cells or unhealthy cells, such as tumor cells. cfDNA shed from tumor cells, commonly referred to as ctDNA in some cases, can be distinguished from cfDNA shed from normal or noncancerous cells using genomic information, such as by identifying genetic variations including mutations and/or structural alterations distinguishing between normal and abnormal cells, as well as additional discriminators such as polynucleotide length, end position, and base modifications (e.g., methylation, hydroxymethylation, formylation, carboxylation, and the like). In some cases, CFNA is shed from cells associated with a fetus into maternal circulation. In some cases, CFNA may originate from a pathogen that has infected a host, such as a subject (e.g., patient).

The term nucleic acid or nucleotide refers to deoxyribonucleic acids (DNA) or ribonucleic acids (RNA) and polymers thereof in either single- or double-stranded form. Unless specifically limited, the term encompasses nucleic acids containing known analogues of natural nucleotides that have comparable properties as the reference nucleic acid. A nucleic acid sequence can comprise combinations of deoxyribonucleic acids and ribonucleic acids. Such deoxyribonucleic acids and ribonucleic acids include both naturally occurring molecules and synthetic analogues. Nucleic acids also encompass all forms of sequences including, but not limited to, single-stranded forms, double-stranded forms, hairpins, stem-and-loop structures, and the like.

The term “mutant” or “variant,” when made in reference to an allele or sequence, generally refers to an allele or sequence that does not encode the phenotype most common in a particular natural population. The terms “mutant allele” and “variant allele” can be used interchangeably. In some cases, a mutant allele can refer to an allele present at a lower frequency in a population relative to the wild-type allele. In some cases, a mutant allele or sequence can refer to an allele or sequence mutated from a wild-type sequence to a mutated sequence that presents a phenotype associated with a disease state and/or drug resistant state. Mutant alleles and sequences may be different from wild-type alleles and sequences by only one base but can be different up to several bases or more. The term mutant when made in reference to a gene generally refers to one or more sequence mutations in a gene, including a point mutation, a SNP, an insertion, a deletion, a substitution, a transposition, a translocation, a copy number variation, or another genetic mutation, alteration, or sequence variation.

The terms “polynucleotide,” “nucleic acid” and “oligonucleotide” are used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cfDNA and cell-free RNA (cfRNA), nucleic acid probes, and primers. A polynucleotide may include one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “standard” or “reference,” as used herein, generally refer to a substance which is prepared to certain pre-defined criteria and can be used to assess certain aspects of, for example, an assay. Standards or references preferably yield reproducible, consistent, and reliable results. These aspects may include performance metrics, examples of which include, but are not limited to, accuracy, specificity, sensitivity, linearity, reproducibility, limit of detection and/or limit of quantitation. Standards or references may be used for assay development, assay validation, and/or assay optimization. Standards may be used to evaluate quantitative and qualitative aspects of an assay. It will be appreciated that standards may be used in any application in which a defined reference is necessary and/or useful. In some aspects, applications may include monitoring, comparing and/or otherwise assessing a QC sample/control, an assay control (product), a filler sample, a training sample, and/or lot-to-lot performance for a given assay.

In general, the term “sequence variant” refers to any variation in sequence relative to one or more reference sequences. Typically, the sequence variant occurs with a lower frequency than the reference sequence for a given population of individuals for whom the reference sequence is known. In some cases, the reference sequence is a single known reference sequence, such as the genomic sequence of a single individual. In some cases, the reference sequence is a consensus sequence formed by aligning multiple known sequences, such as the genomic sequence of multiple individuals serving as a reference population, or multiple sequencing reads of polynucleotides from the same individual. In some cases, the sequence variant occurs with a low frequency in the population (also referred to as a “rare” sequence variant). For example, in non-tissue samples, the sequence variant may occur with a frequency of about or less than about 5%, 4%, 3%, 2%, 1.5%, 1%, 0.75%, 0.5%, 0.25%, 0.1%, 0.075%, 0.05%, 0.04%, 0.03%, 0.02%, 0.01%, 0.005%, 0.001%, or lower. In some non-tissue sample cases, the sequence variant occurs with a frequency of about or less than about 0.1%. In tissue, the sequence variant may occur with a frequency of about or less than about 100%, 90%, 80%, 70%, 60%, 50%, 40%, 30%, 20%, 10%, 5%, or lower. A sequence variant can be any sequence that varies from a reference sequence. A sequence variation may consist of a change in, insertion of, or deletion of a single nucleotide, or of a plurality of nucleotides (e.g., 2, 3, 4, 5, 6, 7, 8, 9, 10, or more nucleotides). Where a sequence variant includes two or more nucleotide differences, the nucleotides that are different may be contiguous with one another, or discontinuous. Non-limiting examples of types of sequence variants include single nucleotide polymorphisms (SNP), deletion/insertion polymorphisms (INDEL), copy number variants (CNV), loss of heterozygosity (LOH), microsatellite instability (MSI), variable number of tandem repeats (VNTR), and retrotransposon-based insertion polymorphisms. Additional examples of types of sequence variants include those that occur within short tandem repeats (STR) and simple sequence repeats (SSR), or those occurring due to amplified fragment length polymorphisms (AFLP) or differences in epigenetic marks that can be detected (e.g., methylation differences). In some aspects, a sequence variant can refer to a chromosome rearrangement, including but not limited to a translocation or fusion gene, or rearrangement of multiple genes resulting from, for example, chromothripsis.

The term “wild type” when made in reference to an allele or sequence, refers to the allele or sequence that encodes the phenotype most common in a particular natural population. In some cases, a wild-type allele can refer to an allele present at highest frequency in the population. In some cases, a wild-type allele or sequence refers to an allele or sequence associated with a normal state relative to an abnormal state, for example a disease state.

Unless defined otherwise, all technical and scientific terms used herein have the same meaning as commonly understood by one of ordinary skill in the art to which this invention belongs. Although any methods and materials similar to or equivalent to those described herein can be used in the practice or testing of the invention, the preferred methods and materials are now described.

### DETAILED DESCRIPTION

The ensuing description provides preferred exemplary embodiments only, and is not intended to limit the scope, applicability, or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

### I. INTRODUCTION

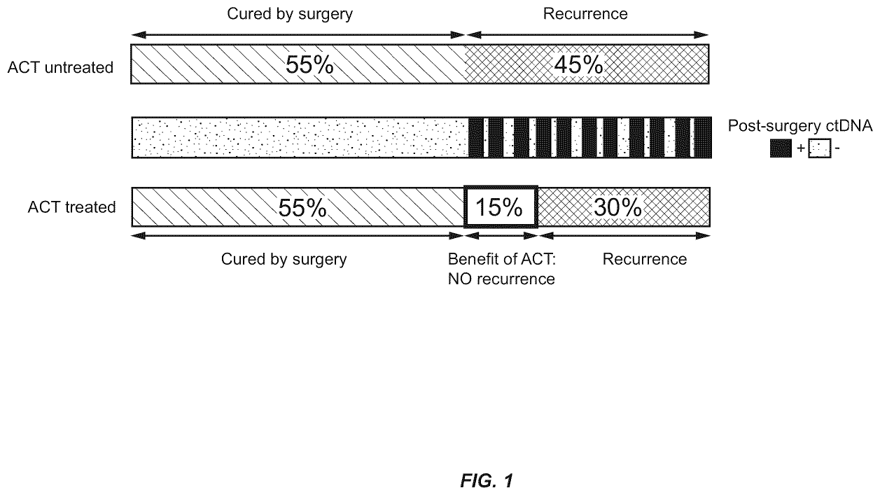

Cancer is a complex group of diseases characterized by the uncontrolled growth and spread of abnormal cells. Advancements in medical science have made it increasingly possible to cure cancer, especially when detected early. Surgery, chemotherapy, radiation therapy, targeted therapy, immunotherapy, and hormone therapy are among many approaches used to treat cancer. In addition to primary approaches to treating cancer (e.g., surgery) secondary therapeutic options are becoming more common when treating cancer patients in an effort to decrease the likelihood of cancer recurrence. One example of this practice is for patients with stage III colon cancer, where standard clinical guidelines recommend performing surgery followed by adjuvant chemotherapy (ACT) as the standard of care. As shown in FIG. 1, recent studies have shown that approximately 50%-55% of the subjects who undergo surgery plus ACT treatment (bottom) would be cured by surgery alone and are therefore receiving ACT unnecessarily. On the other hand, about 30-35% of the subjects who receive ACT may experience a recurrence of the cancer, resulting in only 15-20% of the subjects with stage III colon cancer benefiting from ACT (bottom). Therefore, there is a need for prognostic biomarkers that can identify patients at a higher risk of recurrence that are more likely to benefit from ACT from those who will have a recurrence and not improve from ACT. In so doing, clinicians can reduce the overtreatment of their patients and explore alternative secondary therapeutics that could be more beneficial.

Recent observational and interventional studies in non-metastatic colon cancer have shown that detection of post-surgery cell-free circulating tumor DNA (ctDNA) in blood indicates the presence of minimal residual disease (MRD) and is highly prognostic for development of recurrence of cancer. Hence, ctDNA analysis is a promising approach to guide treatment decisions in stage III colon cancer and other cancers with similar ACT treatment paradigms. Cell-free ctDNA are small random fragments of DNA that break away from the tumor and are found circulating in the person's blood. With respect to post-surgical detection, ctDNA can originate from a small number of cancer cells that may remain in the subject after surgical treatment. Early detection of MRD therefore is crucial for indicating the effectiveness of an initial treatment and for assessing the risk of relapse and tailoring treatment plans accordingly.

Detecting ctDNA in early-stage cancer or in patients with low tumor burden can be challenging due to ctDNA's low abundance, often present at levels of less than 0.10% of total cell free DNA. Furthermore, when evaluating a single landmark timepoint after surgery, radiation therapy, or systemic therapy, the sensitivity for detection of patients who will ultimately relapse can be <50%, as compared to surveillance testing where sensitivity often rises to >80%. Taken together, the clinical data highlights the continued unmet need for technologies to enable detection of ctDNA at low levels for improved clinical sensitivity to identify high-risk patients with early-stage disease who may benefit from additional intervention.

Methods described in the prior art use fixed gene panels with specific genetic alterations, or probes designed to detect specific mutations. Both approaches are restricted in their clinical performance and utility. For example, ctDNA breaks off from the tumor in random, non-predictable fragments, rendering target gene panels and probes useless if the random ctDNA fragment is not complimentary to the sequence detected by the gene panel/probe. Additionally, manufacturing patient-specific bespoke panels for ctDNA detection is also costly, time consuming, and impractical. Achieving high sensitivity without compromising specificity can be challenging with NGS approaches. Current next-generation sequencing (NGS)-based technologies for ctDNA detection rely on analyzing various cell free DNA features to enhance sensitivity. Further, the specificity associated with NGS technologies can also be prone to sequencing errors, background noises, and other artificial errors.

To overcome the challenges faced by current NGS technologies several methods have been attempted. One method is “tumor-uninformed,” where only plasma-derived cfDNA specimens are evaluated for the presence and level of ctDNA. An example of a tumor-uninformed method involves a fixed panel for analysis of sequence alterations and methylation loci. However, due to the lack of a priori knowledge of which specific positions within the tumor are mutated, the sensitivity of this method is dependent upon the alterations being present across the predetermined panel content. Another method is a “tumor-informed” approach; however, it requires patient-specific bespoke panel to be manufactured to detect and quantify ctDNA. This introduces several operational and technical complexities into the assay workflow, mainly prolonged turnaround times of several weeks. There have been attempts to obviate the need for patient-specific panels through development of fixed panels where the content represents common regions altered across specific, pre-specified tumor types, however, these methods are also limited to detecting alterations in the regions included in the panel, which limits the sensitivity.

Attempts to expand fixed panels to the entire human genome have been made; however, these require specialized ctDNA detection algorithms, which have not been fully exploited to maximize analytical sensitivity and specificity. Previously proposed methods to optimize the ctDNA signal to background noise ratio either result in reduction of the actual ctDNA signal or create workflow inefficiencies to maintain analytical performance. Specifically, these methods include inefficient redundant sequencing of independent cfDNA replicates to improve specificity, detection of sufficient somatic alterations to enable analyses of mutational signatures associated with pre-determined mutagenesis processes, and establishment of thresholds for detection of ctDNA based on the observed ctDNA level, which, taken together do not maximize technical performance across the breadth of tumor-specific alterations identified for each patient's tumor.

Other difficulties, problems, and challenges may be associated with the underlying cancer. For example, because colon cancer is a heterogeneous disease, the patient's individual genetic makeup and the location of the tumor makes it difficult to predict the prognosis of ACT. Additionally, prognostic biomarkers for colon cancer may have small effect sizes, making it difficult to identify their significance and predict their impact on patient outcomes. Moreover, the biology of many cancers such as colon cancer is complex and not fully understood, which makes it more challenging to identify and validate prognostic biomarkers. Also, developing and validating prognostic biomarkers can be expensive and time-consuming, which may limit their availability and use in clinical settings.

In order to address and overcome the above-mentioned challenges and others, this disclosure describes an innovative method of detecting cancer using WGS analysis of matched tumor tissue, noncancerous, and non-tissue samples as both test samples and reference samples in the development and implementation of genetic analysis assays to train and evaluate the performance of the assay. High confidence, tumor-specific somatic variants are identified from the patient-matched tumor and noncancerous variant datasets, which are then used to compare to the non-tissue (e.g., plasma) variant dataset through a tumor-informed approach. The non-tissue variants are then filtered and scored through a pretrained machine learning model to determine if circulating tumor DNA (ctDNA) is present (based on variant scores) and the related level within the total cell-free DNA (cfDNA) given the distribution of variant scores observed from a reference cohort. The non-tissue variants and their corresponding variant scores may also be used for other downstream applications.

Because of the challenges described above (sequencing errors, background noises, artifact errors, etc.), using WGS is not an apparent approach because overcoming these fundamental limitations of NGS approaches is not a simple matter. As described, the disclosed method overcomes the challenges of background noise, artifact error, and germline mutations by initial comparing a WGS tumor sample to a WGS noncancerous sample that are both obtained from the same patient. In so doing, a tumor-specific profile is obtained that is free from noise, artifacts, and germline mutations, leaving only somatic tumor-associated mutations. Further, the patient's own tumor-specific mutations are compared to the patient's non-tissue (e.g., plasma) variant profile to generate a patient-specific list of candidate somatic variants. The addition of one or more machine learning models that take advantage of the high-quality candidate somatic variants is also not apparent or previously described in the art. At least one of the machine learning models further filters and generates variant scores for each candidate somatic variant and the variant scores are then used to determine the presence or absence of ctDNA and estimates the ctDNA level. This particular method greatly improves the specificity, sensitivity, and reproducibility of detecting ctDNA and ultimately MRD allowing for even early detection of cancer and thus improved survival outcomes for patients.

### II. COMPUTER ENVIRONMENT

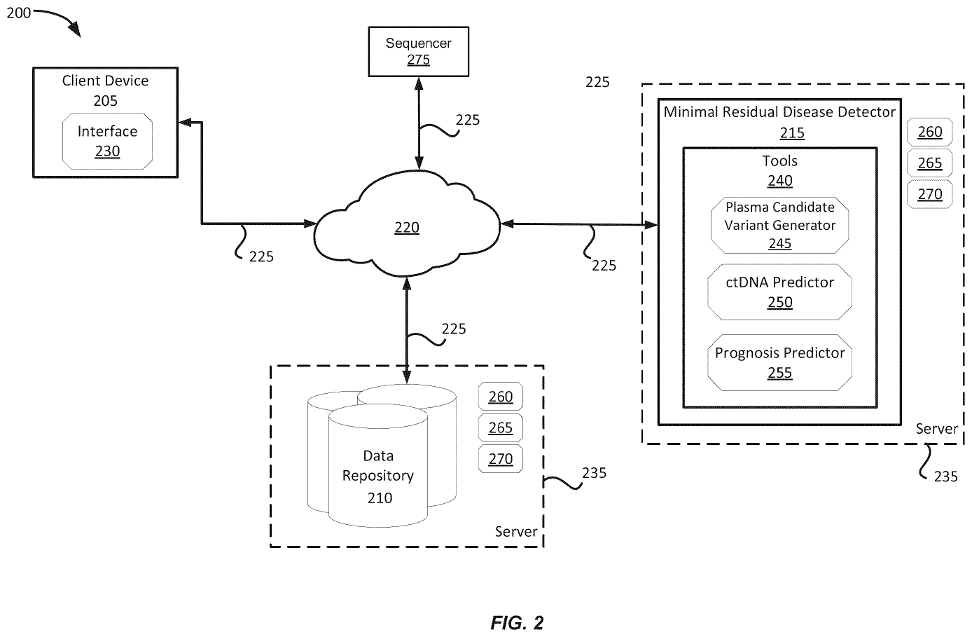

FIG. 2 shows a computing environment 200 in accordance with aspects of the present disclosure. Computing environment 200 includes a client device 205, a data repository 210, a minimal residual disease (MRD) detector platform 215, and a sequencer 275 connected to each other by a network 220. Although FIG. 2 illustrates a particular arrangement of a client device 205, a data repository 210, MRD detector platform 215, and a network 220, this disclosure contemplates any suitable arrangement of a client device 205, a data repository 210, MRD detector platform 215, a sequencer 275, and a network 220. As an example, and not by way of limitation, two or more client devices 205, a data repository 210, MRD detector platform 215, and a sequencer 275 may be connected to each other directly, bypassing network 220. As another example, two or more client devices 205, a data repository 210, a MRD detector platform 215, and a sequencer 275 may be physically or logically co-located with each other in whole or in part. Moreover, although FIG. 2 illustrates a particular number of a client device 205, a data repository 210, a MRD detector platform 215, a sequencer 275, and network 220, this disclosure contemplates any suitable number of client devices 205, data repositories 210, MRD detector platform 215, a sequencer 275, and networks 220. As an example, and not by way of limitation, computing environment 200 may include multiple client devices 205, data repositories 210, MRD detector platforms 215, a sequencer 275, and networks 215.

This disclosure contemplates any type of network 220 familiar to those skilled in the art that may support data communications using any of a variety of available protocols including without limitation TCP/IP (transmission control protocol/Internet protocol), SNA (systems network architecture), IPX (Internet packet exchange), AppleTalk®, and the like. Merely by way of example, network(s) 220 may be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Links 225 may connect a client device 205, a data repository 210, and a MRD detector platform 215 to a network 220 or to each other. This disclosure contemplates any suitable links 225. In particular embodiments, one or more links 225 include one or more wireline (such as for example Digital Subscriber Line (DSL) or Data Over Cable Service Interface Specification (DOCSIS)), wireless (such as for example Wi-Fi or Worldwide Interoperability for Microwave Access (WiMAX)), or optical (such as for example Synchronous Optical Network (SONET) or Synchronous Digital Hierarchy (SDH)) links. In particular embodiments, one or more links 225 each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 225, or a combination of two or more such links 225. Links 225 need not necessarily be the same throughout a computing environment 200. One or more first links 225 may differ in one or more respects from one or more second links 225.

A client device 205 is an electronic device including hardware, software, or embedded logic components or a combination of two or more such components and capable of interacting with the data repository 210 and the MRD detector platform 215 with respect to appropriate product target discovery functionalities in accordance with techniques of the disclosure. The client devices may include several types of computing systems such as portable handheld devices, general purpose computers such as personal computers and laptops, workstation computers, wearable devices, gaming systems, thin clients, various messaging devices, sensors, or other sensing devices, and the like. These computing devices may run various types and versions of software applications and operating systems (e.g., Microsoft Windows®, Apple Macintosh®, UNIX® or UNIX-like operating systems, Linux or Linux-like operating systems such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone), tablets (e.g., iPad®), personal digital assistants (PDAs), and the like. Wearable devices may include Google Glass® head mounted display, and other devices. Client device 205 may be capable of executing various applications such as various Internet-related apps, communication applications (e.g., E-mail applications, short message service (SMS) applications) and may use various communication protocols. This disclosure contemplates any suitable client device 205 configured to generate and output product target discovery content to a user. For example, users may use client device 205 to execute one or more applications, which may generate one or more discovery or storage requests that may then be serviced in accordance with the teachings of this disclosure. A client device 205 may provide an interface 230 (e.g., a graphical user interface) that enables a user of the client device 205 to interact with the client device 205. Client device 205 may also output information to the user via this interface 230. Although FIG. 2 depicts only one client device 205, any number of client devices 205 may be supported.

A data repository 210 is a data storage entity (or sometimes entities) into which data has been specifically partitioned for an analytical or reporting purpose. The data repository 210 may be used to store data and other information for use by the MRD detector platform 215 and client device 205. For example, one or more of the data repositories 210(a) and 210(b) may be used to store data and information to be used as input into the MRD detector platform 215 for generating a prognosis prediction for a patient. In some instances, the data and information relate to various sequencing and variant call files for at least 2 or more samples obtained from the same patient generated by performing WGS. The data may also include any other information used by the MRD detector platform 215 when MRD assay functions. The data repositories 210 may reside in various locations including servers 235. For example, a data repository used by server 235 may be local to server 235 or may be remote from server 235 and in communication with server 235 via a network-based or dedicated connection of network 220. Data repositories 210(a) and 210(b) may be of distinct types or of the same type. In certain examples, a data repository may be a database which is an organized collection of data stored and accessed electronically from one or more storage devices such as one or more servers 235. The one or more servers 235 may be configured to execute a database application that provides database services to other computer programs or to computing devices (e.g., client device 205 and MRD detector platform 215) within the computing environment, as defined by a client-server model. One or more of these databases may be adapted to enable storage, update, and retrieval of data to and from the database in response to SQL-formatted commands or like programming language that is used to manage databases and perform various operations on the data within them.

The MRD detector platform 215 comprises a set of tools 240 for analyzing and visualizing data (i.e., data stored in data repository 210). The MRD detector platform 215 is used to execute a process to identify high-risk patients with early-stage disease, such as those with MRD and predict whether the patient will benefit from a secondary treatment therapeutic. In the configuration depicted in FIG. 2, the set of tools 240 includes three processors: a candidate somatic variant generator 245, a ctDNA predictor 250, and a prognosis predictor 255. The candidate somatic variant generator 245 is responsible for loading, processing, and saving data accessed from the data repository 210 to be used by the candidate somatic variant generator 245 itself, by the ctDNA predictor 250, and/or the prognosis predictor 255. The ctDNA predictor 250 uses the processed data (e.g., high confident candidate somatic variant calls) from the candidate somatic variant generator 245 to generate variant scores for the candidate somatic variants, classify a patient sample (e.g., non-tissue such a plasma) as ctDNA+ or ctDNA−, and/or estimate a ctDNA level in a patient sample. The prognosis predictor 255 uses the candidate somatic variant scores generated by ctDNA predictor 250 and outputs predictions related to whether the patient has a low or high-risk of cancer recurrence and whether the patient will benefit from a disease therapy. The MRD detector platform 215 may reside in various locations including servers 235. For example, MRD detector platform 215 used by server 235 may be local to server 235 or may be remote from server 235 and in communication with server 235 via a network-based or dedicated connection of network 220. The MRD detector platform 215 may be of different configurations or of the same configuration. The one or more servers 235 may be configured to execute a discovery application that provides discovery services to other computer programs or to computing devices (e.g., client device 205) within the computing environment, as defined by a client-server model.

In various instances, server 235 may be adapted to run one or more services or software applications that enable one or more embodiments described in this disclosure. In certain instances, server 235 may also provide other services or software applications that may include non-virtual and virtual environments. In some examples, these services may be offered as web-based or cloud services, such as under a Software as a Service (SaaS) model to the users of client device 205. Users operating client device 205 may in turn utilize one or more client applications to interact with server 235 to utilize the services provided by these components (e.g., database and rescue applications). In the configuration depicted in FIG. 2, server 235 may include one or more components 260, 265, and 270 that implement the functions performed by server 235. These components may include software components that may be executed by one or more processors, hardware components, or combinations thereof. It should be appreciated that multiple different device configurations are possible, which may be different from computing environment 200. The example shown in FIG. 2 is thus one example of a computing environment (e.g., a distributed system for implementing an example computing system) and is not intended to be limiting.

Server 235 may be composed of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. Server 235 may include one or more virtual machines running virtual operating systems, or other computing architectures involving virtualization such as one or more flexible pools of logical storage devices that may be virtualized to maintain virtual storage devices for the server. In various instances, server 235 may be adapted to run one or more services or software applications that provide the functionality described in the foregoing disclosure.

The computing systems in server 235 may run one or more operating systems including any of those discussed above, as well as any commercially available server operating system. Server 235 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, Sybase®, IBM® (International Business Machines), and the like.

In some implementations, server 235 may include one or more applications to analyze and consolidate data feeds and/or data updates received from users of client computing devices 205. As an example, data feeds and/or data updates may include, but are not limited to, in vivo feeds, in silico feeds, or real-time updates received from public studies, user studies, one or more third party information sources, and data streams (continuous, batch, or periodic), which may include real-time events related to sensor data applications, biological system monitoring, and the like. Server 235 may also include one or more applications to display the data feeds, data updates, and/or real-time events via one or more display devices of client computing devices 205.

Sequencer 275 is a sequencing device which is any machine capable of sequencing one or more nucleic acid molecules to generate raw sequencing data (e.g., reads). Library prepared nucleic acid samples may be pooled and loaded into lanes of a sequencing flow cell. The flow cell may be loaded into sequencer 275 and imaged to generate sequence data. For example, reagents that interact with the nucleic acid samples fluoresce at particular wavelengths in response to an excitation beam and thereby return a signal for imaging. For instance, the fluorescent components may be generated by fluorescently tagged nucleic acids that hybridize to complementary molecules of the components or to fluorescently tagged nucleotides that are incorporated into an oligonucleotide using a polymerase. As will be appreciated by those skilled in the art, the wavelength at which the dyes of the sample are excited and the wavelength at which they fluoresce will depend upon the absorption and emission spectra of the specific dyes. Sequencer 275 may optionally include or be operably coupled to its own dedicated sequencer computer with its own input/output mechanisms, one or more processors, and memory. Additionally or alternatively, sequencer 275 may be operably coupled to a server 235 or client device 205 via network 220. Client device 205 may access the raw sequencing data files from data repositories 210 and execute instructions for analyzing or communicating the sequence data to network 220.

### III. WORKFLOW FOR DETECTING CTDNA IN A NON-TISSUE SAMPLE

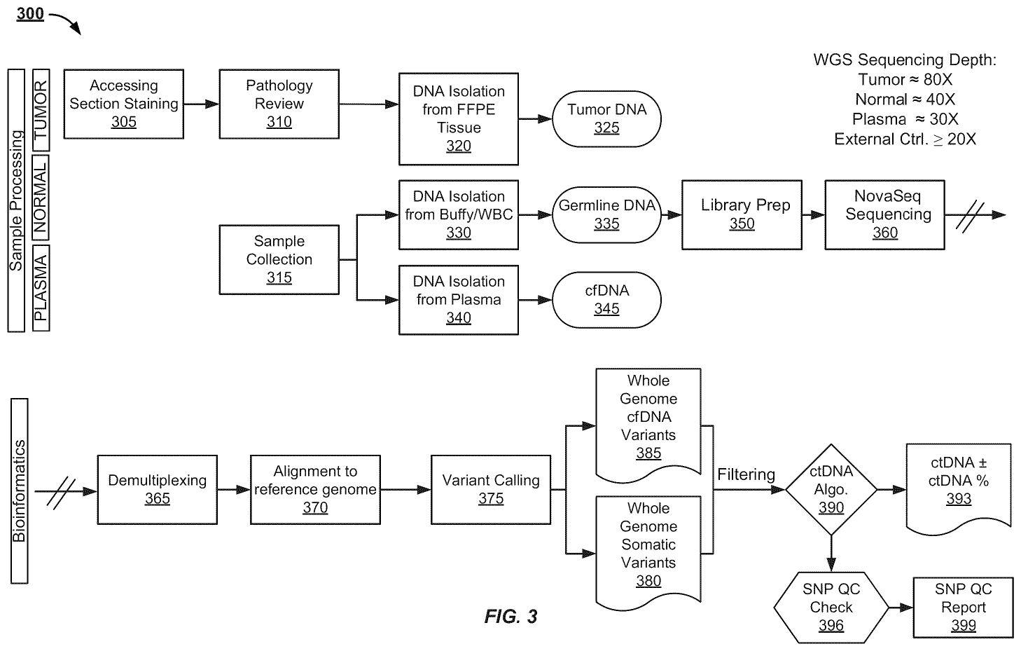

FIG. 3 shows an exemplary sample processing and computational workflow 300 for detecting cancer using WGS data to address the limitations of current technologies. The computational portion of workflow 300 (e.g., equivalent to candidate somatic variant generator 245 with respect to FIG. 2) analyzes WGS data to enable detection of ctDNA at low levels, thereby providing improved clinical sensitivity. Briefly, the sample processing workflow comprises accessing/obtaining samples (e.g., tumor, normal (noncancerous), and non-tissue samples), DNA isolation, library preparation, and sequencing. The experimental procedures may be performed in a laboratory by qualified research personnel, while the bioinformatic procedures may be performed on a client (e.g., researcher, clinician, and the like) electronic device that includes hardware, software, or embedded logic components or a combination of two or more such components. The client devices may include several types of computing systems such as portable handheld devices, general purpose computers such as personal computers and laptops, workstation computers, wearable devices, gaming systems, thin clients, various messaging devices, sensors, or other sensing devices, and the like. These computing devices may run various types and versions of software applications and operating systems (e.g., Microsoft Windows®, Apple Macintosh®, UNIX® or UNIX-like operating systems, Linux or Linux-like operating systems such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone), tablets (e.g., iPad®), personal digital assistants (PDAs), and the like. Wearable devices may include Google Glass® head mounted display, and other devices. The client device may be capable of executing various applications such as various Internet-related apps, communication applications (e.g., E-mail applications, short message service (SMS) applications) and may use various communication protocols. This disclosure contemplates any suitable client device configured to perform the bioinformatic workflow described in FIG. 3.

### Obtaining Samples

In the sample processing portion of workflow 300 (top), at least two or more samples are obtained from a single patient. The sample, or biological sample can be a cell-containing liquid or a tissue. The sample can comprise, but is not limited to, amniotic fluid, tissue biopsies, blood, blood cells, bone marrow, fine needle biopsy samples, peritoneal fluid, amniotic fluid, plasma, pleural fluid, saliva, semen, serum, tissue, or tissue homogenates, frozen or paraffin sections of tissue. Methods of obtaining the specimen include, but are not limited to, biofilms, aspirations, tissue sections, swabs, drawing blood or other fluids, surgical or needle biopsies, and the like. The at least two or more samples obtained from the same patient may be nucleic acid samples (e.g., DNA and/or RNA in both natural and synthetic forms).

The sample can be obtained from a noncancerous subject or a subject with a disease (e.g., solid tumor malignancies). As shown in FIG. 3 the at least two or more samples may be a tumor sample (e.g., cancer positive sample), a normal sample (any bodily tissue or fluid containing nucleic acid that is generally cancer-free (e.g., lymphocytes, saliva, buccal cells, or other tissues and fluids)), and a non-tissue sample. All samples (e.g., tumor, normal, and non-tissue) are collected from the same patient. Although FIG. 3 illustrates patient samples as specifically including plasma samples, additional cfDNA non-tissue samples may be contemplated such as sputum, saliva, cerebral spinal fluid, surgical drain fluid, urine, cyst fluid, to name a few non-limiting examples. In some instances, only two samples may be collected (e.g., a tumor sample and a whole blood sample) because the plasma sample may be isolated from the blood sample leaving white blood cells, for example, as the normal cancer-free sample. In other instances, more than two samples are collected from the same patient, for example three samples that include a tumor sample, a normal sample (e.g., a cancer-free sample obtained from any tissue or fluid), and a whole blood sample for plasma isolation. The tumor sample and/or the normal sample can be tissue samples or body fluid samples. Additionally, more than one whole blood sample may be collected from the patient at a single timepoint or across multiple timepoints, such as over the course of treatment. For example, at a first appointment, at least two whole blood samples may be collected. Then at a second and subsequent timepoints, one or more whole blood samples may be collected.

The tumor sample may be obtained as a formalin-fixed paraffin-embedded (FFPE) sample (e.g., tissue) that is previously prepared. A portion of the FFPE tumor sample, prior to DNA isolation, may first be section and stained 305 in a tissue pathology lab (or any other lab suitable for tissue preparations and staining). The processes of tissue/cell fixation, embedding, sectioning, staining, and imaging are well known in the art and any appropriate method may be used. Briefly, a sample (e.g., tissue) may first be fixed with a fixing agent to preserve the sample and slow down degradation. The fixed sample may then be embedded with, for example, paraffin, in preparation for tissue sectioning. The fixed and/or embedded sample may be sectioned into slices using, for example, a cryostat into appropriately thick sections. The sectioned sample is mounted on a slide where various staining methods may be performed to render relevant structures more visible. Examples of staining methods that may be used include histopathological staining methods, histochemical methods, hematoxylin and eosin (H&E) staining, trichrome stains, periodic acid-Schiff, silver stains, iron stains, immunohistochemistry (IHC), etc.

Following sectioning and staining 305 of the tumor sample, the stained image is reviewed/analyzed by a pathologist 310. The pathologist may review and manually annotate the sample by indication features of interest (e.g., tissue degeneration, tissue damage, cancer positive/negative etc.). If the tumor sample is considered acceptable after pathology review 310, the tumor sample may be sent for experimental processing, such as DNA isolation.

As described above, the normal sample and the non-tissue sample (e.g., plasma) may be collected 315 from a single sample or from multiple samples collected from the same patient as the tumor sample. As an example of single sample collection, a whole blood sample can be collected from the patient using venipuncture of other routine methods known in the art. By way of example, and without limitation, the non-tissue sample can be a plasma sample. Plasma is separated from a blood sample by adding an anticoagulant to the blood sample and centrifuging the blood sample at sufficient speed to separate the plasma from the blood cells. The plasma sample can include nucleic acids (e.g., cell-free DNA, ctDNA) associated with a patient's MRD. The remaining fraction that is separated from the plasma comprises blood cells (e.g., white blood cells (monocytes, lymphocytes, neutrophils, eosinophils, basophils, and macrophages), red blood cells (erythrocytes), platelets, and a buffy coat fraction (e.g., includes leukocytes and thrombocytes), all of which may be used as the normal sample. As an example of when the normal sample and the non-tissue sample (e.g., plasma) are collected from different biological samples from the same patient, the normal sample may be any bodily tissue or fluid containing nucleic acid considered generally cancer-free. The non-tissue sample can be collected from any biological sample that includes cell free DNA and/or ctDNA such as plasma, sputum, saliva, cerebral spinal fluid, surgical drain fluid, urine, cyst fluid, to name a few non-limiting examples.

In certain embodiments, tumor samples may include, for example, cell-free nucleic acid (including DNA or RNA) or nucleic acid isolated from a tumor tissue sample such as biopsied or resected tissue. Normal samples, in certain aspects, may include nucleic acid isolated from any non-tumor tissue of the patient, including, for example, patient lymphocytes or cells obtained via buccal swab. Cell-free nucleic acids may be fragments of DNA or ribonucleic acid (RNA) present in a patient's blood stream. For example, the circulating cell-free nucleic acid is one or more fragments of DNA obtained from a non-tissue sample (e.g., plasma, saliva, urine, etc.) of the patient.

As described herein, “patient,” and “subject” are used interchangeably and refer to a mammal, such as a human or non-human primate, wherein the mammalian subject can be of any age. In any of the methods set forth herein, the subject can be suspected of having a disease, diagnosed with a disease, or receiving treatment for a disease. For example, the subject may be suspected of having cancer, may be diagnosed with cancer, or is receiving treatment for cancer. In one embodiment, the subject may be suspected of having colon cancer, may be diagnosed with colon cancer, or is receiving treatment for colon cancer. Subjects may also include living humans that are receiving medical care for a disease or condition. This includes people with no defined illness who are being investigated for signs of disease. In some embodiments, the patient has received surgery to remove a cancer tumor (e.g., a colon cancer tumor) and may or may not have received ACT post-surgery. In other embodiments, post-surgical ctDNA may be detected indicating the presence of MRD, which is a strong prognostic factor for cancer.

Once at least two or more samples are collected from the same patient, the samples are ready for DNA isolation. DNA is isolated from the FFPE tumor tissue sample 320 to generate purified tumor DNA 325, DNA may be isolated from the buffy fraction or white blood cells (WBC) 330 layer of a blood sample to generate purified germline DNA 335, and DNA may be isolated from the plasma 340 layer of a blood sample to generate cfDNA 345. The germline DNA 335 is the normal, noncancerous sample. In some instances, the normal (germline) DNA 335 and the plasma cfDNA 345 are not collected from the same sample (e.g., same whole blood collection) and may instead be collected from two different samples collected from the same patient. For example, germline DNA 335 can be collected from any biological sample considered to be generally cancer free while cfDNA 345 can be collected from any biological sample considered to comprise cfDNA and/or ctDNA such as plasma, sputum, saliva, cerebral spinal fluid, surgical drain fluid, urine, cyst fluid, etc.

Various methods are known in the art for isolating DNA from a sample (e.g., cells, tissue, non-tissue, etc.) One method for isolating DNA may include the using a reagent kit (e.g., tubes and DNA extraction reagents, etc.). The kit may include tools for library preparation such as probes for hybrid capture as well as any useful reagents & protocols for fragmentation, adapter ligation, purification/isolation, etc. Using kits or other techniques known in the art a sample containing DNA is obtained. Other methods for isolating/extracting DNA from a sample involve disruption and lysis of the starting material followed by the removal of proteins and other contaminants and finally recovery of the DNA. Cell lysis procedures and reagents are known in the art and may generally be performed by chemical (e.g., detergent, hypotonic solutions, enzymatic procedures, and the like), physical (e.g., French press, sonication, and the like), or electrolytic lysis methods. Removal of proteins can be achieved, for example, by digestion with proteinase K, followed by salting-out, organic extraction, gradient separation, or binding of the DNA to a solid-phase support (either anion-exchange or silica technology). DNA may be recovered by precipitation using ethanol or isopropanol. The choice of method depends on many factors including, for example, the amount of sample, the required quantity and molecular weight of the DNA, the purity required for downstream applications, and the time and expense. The sample DNA isolated/extracted may be whole genomic DNA, circulating cell-free DNA, ctDNA, mitochondrial DNA, circular DNA, and the like. As shown in FIG. 3, tumor DNA 325 and germline DNA 335 are whole genomic DNA samples while the DNA isolated from non-tissue (e.g., plasma) is cfDNA 345. The amount of DNA isolated from a sample can depend on several factors such as sample type (tissue versus cells versus low concentrated cfDNA) sample size, sample quality, etc. DNA isolation from a tumor tissue sample can yield at least 200 ng of DNA. DNA isolated from a normal sample can yield at least 50 ng of DNA and DNA isolated from a non-tissue sample (e.g., plasma) can yield at least 10 ng of cfDNA. In some instances, to ensure sufficient (e.g., at least 10 ng) of cfDNA is isolated, more than one whole blood sample is collected from the patient and the DNA isolated from the more than one whole blood samples is pooled. For example, at least 2 10 mL volumes of whole blood are collected from the patient to obtain sufficient plasma to isolate at least 10 ng of cfDNA from.

Other examples for isolating DNA from tumor, normal, and non-tissue samples may further include the QIAmp system from Qiagen (Venlo, Netherlands); the Triton/Heat/Phenol protocol (THP); a blunt-end ligation-mediated whole genome amplification (BL-WGA); or the NucleoSpin system from Macherey-Nagel, GmbH & Co.KG (Duren, Germany). See Xue, 2009, Optimizing the yield and utility of circulating cell-free DNA from plasma and serum, Clin Chim Acta 404(2):100-104. Also see Li, 2006, Whole genome amplification of plasma-circulating DNA enables expanded screening for allelic imbalances in plasma, J Mol Diag 8(1):22-30. Both are incorporated by reference.

In some instances, when it is determined that there is an insufficient amount of nucleic acid for analysis, amplification may be used to increase the amount of nucleic acid. Amplification refers to production of additional copies of a nucleic acid sequence and is generally carried out using polymerase chain reaction (PCR) or other technologies known in the art (e.g., Dieffenbach and Dveksler, PCR Primer, a Laboratory Manual, 1995, Cold Spring Harbor Press, Plainview, NY). PCR refers to methods by K. B. Mullis (U.S. Pat. Nos. 4,683,195 and 4,683,202, hereby incorporated by reference) for increasing concentration of a segment of a nucleic acid sequence in a mixture of genomic DNA without cloning or purification.

### Whole Genome Sequencing

All three obtained nucleic acid samples (e.g., tumor, normal, and non-tissue) are sequenced using any suitable whole genome sequencing (WGS) methods. The nucleic acids may be amplified before sequencing. Sequencing data is obtained from the WGS, and the sequencing data comprises sequence reads.

### 1. DNA Fragmentation and Library Preparation

Following DNA isolation, the isolated DNA (tumor 325, germline 335, and cfDNA 345) undergoes library preparation 350. Whole genomic DNA (tumor 325 and germline 335) are fragmented into a plurality of shorter double stranded DNA target fragments, while cfDNA from non-tissue samples may not be fragmented. In general, fragmentation of DNA may be performed physically, or enzymatically. For example, physical fragmentation may be performed by acoustic shearing, sonication, microwave irradiation, or hydrodynamic shear. Acoustic shearing and sonication are the main physical methods used to shear DNA. For example, the Covaris® instrument (Woburn, MA) is an acoustic device for breaking DNA into 100 bp-5 kb. Covaris also manufactures tubes (gTubes) which will process samples in the 6-20 kb for Mate-Pair libraries. Another example is the Bioruptor® (Denville, NJ), a sonication device utilized for shearing chromatin, DNA and disrupting tissues. Small volumes of DNA can be sheared to 150 bp-1 kb in length. The Hydroshear® from Digilab (Marlborough, MA) is another example and utilizes hydrodynamic forces to shear DNA. Nebulizers, such as those manufactured by Life Technologies (Grand Island, NY) can also be used to atomize liquid using compressed air, shearing DNA into 100 bp-3 kb fragments in seconds. As nebulization may result in loss of sample, in some instances, it may not be a desirable fragmentation method for limited quantities samples. Sonication and acoustic shearing may be better fragmentation methods for smaller sample volumes because the entire amount of DNA from a sample may be retained more efficiently. Other physical fragmentation devices and methods that are known or developed can also be used.

Various enzymatic methods may also be used to fragment DNA. For example, DNA may be treated with DNase I, or a combination of maltose binding protein (MBP)-T7 Endo I and a non-specific nuclease such as Vibrio vulnificus nuclease (Vvn). The combination of non-specific nuclease and T7 Endo synergistically work to produce non-specific nicks and counter nicks, generating fragments that disassociate 8 nucleotides or less from the nick site. In another example, DNA may be treated with NEBNext® dsDNA Fragmentase® (NEB, Ipswich, MA). NEBNext® dsDNA Fragmentase generates dsDNA breaks in a time-dependent manner to yield 50-1,000 bp DNA fragments depending on reaction time. NEBNext dsDNA Fragmentase contains two enzymes, one randomly generates nicks on dsDNA and the other recognizes the nicked site and cuts the opposite DNA strand across from the nick, producing dsDNA breaks. The resulting DNA fragments contain short overhangs, 5′-phosphates, and 3′-hydroxyl groups.

In some instances, the whole genomic DNA samples are fragmented into specific size ranges of target fragments. For example, whole genomic DNA samples may be fragmented into fragments in the range of about 25-100 bp, about 25-150 bp, about 50-200 bp, about 25-200 bp, about 50-250 bp, about 25-250 bp, about 50-300 bp, about 25-300 bp, about 50-500 bp, about 25-500 bp, about 150-250 bp, about 100-500 bp, about 200-800 bp, about 500-1300 bp, about 750-2500 bp, about 1000-2800 bp, about 500-3000 bp, about 800-5000 bp, or any other size range within these ranges. For example, the whole genomic DNA samples may be fragmented into fragments of about 300-800 bp. In some instances, the fragments may be larger or smaller by about 25 bp. After fragmentation, DNA fragments may be blunt ended.

Using the DNA fragments (or unfragmented cfDNA) generated in the above-described process, a DNA library is prepared. A DNA library is a plurality of polynucleotide molecules (e.g., a sample of nucleic acids) that are prepared, assembled and/or modified for a specific process, non-limiting examples of which include immobilization on a solid phase (e.g., a solid support, a flow cell, a bead), enrichment, amplification, cloning, detection and/or for nucleic acid sequencing. A DNA library can be prepared prior to or during a sequencing process. A DNA library (e.g., sequencing library) can be prepared by a suitable method as known in the art. A DNA library can be prepared by a targeted or a non-targeted preparation process.

A DNA library is modified to comprise one or more polynucleotides of known composition, non-limiting examples of which include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, the like or combinations thereof. Polynucleotides of known sequence can be added at a suitable position, for example on the 5′ end, 3′ end or within a nucleic acid sequence. Polynucleotides of known sequence can be the same or different sequences. In some embodiments, a polynucleotide of known sequence is configured to hybridize to one or more oligonucleotides immobilized on a surface (e.g., a surface in flow cell). For example, a nucleic acid molecule comprising a 5′ known sequence may hybridize to a first plurality of oligonucleotides while the 3′ known sequence may hybridize to a second plurality of oligonucleotides. A DNA library can comprise chromosome-specific tags, capture sequences, labels and/or adapters. A DNA library can comprise one or more detectable labels. One or more detectable labels may be incorporated into a DNA library at a 5′ end, at a 3′ end, and/or at any nucleotide position within a nucleic acid in the library. A DNA library can comprise hybridized oligonucleotides that are labeled probes that may be added prior to immobilization on a solid phase.

A ligation-based library preparation method is used (e.g., ILLUMINA TRUSEQ, Illumina, San Diego Calif). Ligation-based library preparation methods often make use of an adapter design which can incorporate an index sequence (e.g., a sample index sequence to identify sample origin for a nucleic acid sequence) at the initial ligation step and often can be used to prepare samples for single-read sequencing, paired-end sequencing, and multiplexed sequencing. For example, nucleic acids (e.g., fragmented or unfragmented nucleic acids) may be end repaired by a fill-in reaction, an exonuclease reaction, or a combination thereof. The resulting blunt-end repaired nucleic acid can then be extended by a single nucleotide, which is complementary to a single nucleotide overhang on the 3′ end of an adapter/primer. Any nucleotide can be used for the extension/overhang nucleotides.

DNA library preparation comprises ligating an adapter oligonucleotide to the sample DNA fragments or ctDNA. The adapter sequences are attached to the template nucleic acid molecule with an enzyme. The enzyme may be a ligase or a polymerase. The ligase may be any enzyme capable of ligating an oligonucleotide (RNA or DNA) to the template nucleic acid molecule. Suitable ligases include T4 DNA ligase and T4 RNA ligase, available commercially from New England Biolabs (Ipswich, MA). Methods for using ligases are well known in the art. The polymerase may be any enzyme capable of adding nucleotides to the 3′ and the 5′ terminus of template nucleic acid molecules.

Adapter oligonucleotides are often complementary to flow-cell anchors, and sometimes are utilized to immobilize a nucleic acid library to a solid support, such as the inside surface of a flow cell, for example. An adapter oligonucleotide may comprise an identifier, one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). An adapter oligonucleotide may comprise one or more of primer annealing polynucleotide (e.g., for annealing to flow cell attached oligonucleotides and/or to free amplification primers), an index polynucleotide (e.g., sample index sequence for tracking nucleic acid from different samples, also referred to as a sample ID), and a barcode polynucleotide (e.g., single molecule barcode (SMB) for tracking individual molecules of sample nucleic acid that are amplified prior to sequencing; also referred to as a molecular barcode). A primer annealing component of an adapter oligonucleotide comprises one or more universal sequences (e.g., sequences complementary to one or more universal amplification primers). An index polynucleotide (e.g., sample index; sample ID) is a component of an adapter oligonucleotide and/or a component of a universal amplification primer sequence.

Adapter oligonucleotides may be used in combination with amplification primers (e.g., universal amplification primers) to generate library constructs comprising one or more of universal sequences, molecular barcodes, sample ID sequences, spacer sequences, and a sample nucleic acid sequence. Adapter oligonucleotides, when used in combination with universal amplification primers, are designed to generate library constructs comprising an ordered combination of one or more of universal sequences, molecular barcodes, sample ID sequences, spacer sequences, and a sample nucleic acid sequence. For example, a library construct may comprise a first universal sequence, followed by a second universal sequence, followed by first molecular barcode, followed by a spacer sequence, followed by a template sequence (e.g., sample nucleic acid sequence), followed by a spacer sequence, followed by a second molecular barcode, followed by a third universal sequence, followed by a sample ID, followed by a fourth universal sequence. Additionally or alternatively, adapter oligonucleotides, when used in combination with amplification primers (e.g., universal amplification primers), are designed to generate library constructs to differentiate each strand of a template molecule (e.g., sample nucleic acid molecule). In some cases, adapter oligonucleotides are duplex adapter oligonucleotides.

A universal sequence is a specific nucleotide sequence that is integrated into two or more nucleic acid molecules or two or more subsets of nucleic acid molecules where the universal sequence is the same for all molecules or subsets of molecules that it is integrated into. A universal sequence is often designed to hybridize to and/or amplify a plurality of different sequences using a single universal primer that is complementary to a universal sequence. Two (e.g., a pair) or more universal sequences and/or universal primers may be used. A universal primer often comprises a universal sequence. In some instances, one or more universal sequences are used to capture, identify and/or detect multiple species or subsets of nucleic acids.

Optionally, the DNA library, or parts thereof, are amplified (e.g., amplified by a PCR-based method). For example, a sequencing method may comprise amplification of a DNA library. A DNA library can be amplified prior to or after immobilization on a bead or solid support (e.g., a solid support in a flow cell). Nucleic acid amplification includes the process of amplifying or increasing the numbers of a nucleic acid template and/or of a complement thereof that are present (e.g., in a nucleic acid library), by producing one or more copies of the template and/or its complement. Amplification can be carried out by a suitable method. A DNA library can be amplified by a thermocycling method, by an isothermal amplification method, or a rolling circle amplification method. In certain sequencing methods, a DNA library is added to a flow cell and immobilized by hybridization to anchors under suitable conditions. This type of nucleic acid amplification is often called solid phase amplification. During solid phase amplification, all, or a portion of, the amplified products are synthesized by an extension initiating from an immobilized primer. Solid phase amplification reactions are analogous to standard solution phase amplifications except that at least one of the amplification oligonucleotides (e.g., primers) is immobilized on a solid support. In some instances, modified nucleic acids (e.g., nucleic acid modified by addition of adapters) are amplified.

### 2. Whole Genome Sequencing

The library prepped nucleic acids (e.g., tumor, normal, cfDNA) are sequenced 360 using a machine capable of sequencing nucleic acids (e.g., sequencer 275 described with respect to FIG. 2). Examples of sequencing may include, without limitation, NovaSeq, HiSeq, Genome Analyzer IIx, MiSeq, HiScanSQ, 454 DNA sequencer, GS FLX+, GS Junior System, OLiD next-generation sequencing platform, Ion PGM System, Ion Proton System, Ion S5, Ion S5xl, CEQ 8000, RS system, Sequel system, nanopore sequencers, DNBSEQ-G50, DNBSEQ-G400, DNBSEQ-T7, Ultima Genomics UG100, etc. In certain instances, a full or substantially full sequence is obtained and sometimes a partial sequence is obtained.

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2^{nd} and 3^{rd} generation) sequencing techniques capable of sequencing DNA in a massively parallel fashion can be used for methods described herein and are collectively referred to herein as “massively parallel sequencing” (MPS). In certain embodiments, a non-targeted approach is used where most or all nucleic acids in a sample are sequenced, amplified and/or captured randomly.

Other suitable sequencing technologies may include single molecule, real-time (SMRT) technology of Pacific Biosciences (in SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW) where the fluorescent label is excited and produces a fluorescent signal, and the fluorescent tag is cleaved off. Detection of the corresponding fluorescence of the dye indicates which base was incorporated); nanopore sequencing (DNA is passed through a nanopore and each base is determined by changes in current across the pore, as described in Soni & Meller, 2007, Progress toward ultrafast DNA sequence using solid-state nanopores, ClinChem 53(11):1996-2001); chemical-sensitive field effect transistor (chemPET) array sequencing (e.g., as described in U.S. Pub. 2009/0026082); and electron microscope sequencing (as described, for example, by Moudrianakis, E. N. and Beer M., in Base sequence determination in nucleic acids with the electron microscope, III. Chemistry and microscopy of guanine-labeled DNA, PNAS 53:564-71 (1965).

In some embodiments, WGS is performed on the prepared DNA library samples. WGS is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, an image is captured, and the identity of the first base is recorded. The 3′ terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

The WGS method described above may sequence samples at different depths. For example, WGS may be performed at a depth of 80× for the tumor DNA samples 325, a depth of 40× for the normal (e.g., germline) DNA samples 335, a depth of 30× for the non-tissue cfDNA samples 345, and a depth of greater than or equal to 20× for external control samples.

Sequencing methods (e.g., WGS) generate a large number of reads. As used herein, “reads” (e.g., “a read,” “a sequence read”) are short nucleotide sequences produced by any sequencing process described herein or known in the art. Reads can be generated from one end of nucleic acid fragments (“single-end reads”), and sometimes are generated from both ends of nucleic acid fragments (e.g., paired-end reads, double-end reads). The length of a sequence read is often associated with the particular sequencing technology. High-throughput methods, for example, provide sequence reads that can vary in size from tens to hundreds of base pairs (bp). Sequencing reads may have a mean, median, average, or absolute length of about 15 bp to about 1000 bp. For example, sequencing reads may be about 15 bp, 16 bp, 17 bp, 18 bp, 19 bp, 20 bp, 25 bp, 50 bp, 100 bp, 150 bp, 200 bp, 300 bp, 400 bp, 500 bp, 600 bp, 700 bp, 800 bp, 900 bp, or about 1000 bp or about any integer value between 15 bp and 1000 bp. Sequencing reads, and their associated quality scores, are stored in files known as FASTQ files or FASTA files. Typically, FASTQ files can comprise about 1 million to about 5 million reads per sample; however, more or less reads may be generated depending on the sample. Nonlimiting examples can include: (i) FASTQ files for tumor samples can include about 2 billion reads to about 4 billion reads per sample, (ii) FASTQ files for normal (noncancerous) samples can include about 800 million reads to about 1.5 billion reads per sample, and (iii) FASTQ files for non-tissue (e.g., plasma) samples can include about 800 million reads to about 2 billion reads per sample.

### 3. Bioinformatics Workflow

In some embodiments, sequence reads are generated, obtained, gathered, assembled, manipulated, transformed, processed, and/or provided by a sequence subsystem. A machine comprising a sequence subsystem can be a suitable machine and/or apparatus that determines the sequence of a nucleic acid utilizing a sequencing technology known in the art. In some embodiments a sequence subsystem can align, assemble, fragment, complement, reverse complement, and/or error check (e.g., error correct sequence reads). The sequence reads are processed using a sequence processing subsystem to obtain sequence read data. The processing of the sequence reads includes read alignment, mapping, and filtering. To perform all these processing steps, the bioinformatics workflow comprises steps including demultiplexing 365, reference genome alignment 370, variant calling 375 to identify whole genome cfDNA variants 380 and whole genome somatic variants 385, a ctDNA algorithm 390, and ctDNA percentage values 395.

As described above, the outputs of sequencing are FASTQ files that comprise all the reads for a single sample. Part of the process of generating FASTQ files is demultiplexing 365 (e.g., sorting) all the different library samples that were pooled together in a single flow cell lane into their own FASTQ file. In a typical WGS sequencing run, multiple library samples (e.g., 4, 12, 16, etc.) are combined and loaded onto a single lane of a sequencing flow cell. Because during the library preparation, each DNA fragment in a sample had a corresponding unique barcode ligated onto the fragments. Accordingly, when multiple libraries are pooled for sequencing, the barcodes allow for the samples to be distinguished from one another. The barcodes are also what are used to sort each sample into its own sequencing FASTQ file (i.e., demultiplexing 365).

Alignment of reads to a reference genome (e.g., a human reference genome) 370 involves mapping any number of reads to a specified nucleic acid region (e.g., a chromosome or portion thereof) and are referred to as counts. As used herein, the term “reference genome” can refer to any known, sequenced or characterized genome, whether partial or complete, of any organism or virus which may be used to reference identified sequences from a subject. For example, a reference genome used for human subjects as well as many other organisms can be found at the National Center for Biotechnology Information at World Wide Web URL ncbi.nlm.nih.gov.

Any suitable mapping/alignment method (e.g., process, algorithm, program, software, subsystem, the like or combination thereof) can be used. Non-limiting examples of computer algorithms that can be used to align sequences include, without limitation, BLAST, BLITZ, FASTA, BOWTIE 1, BOWTIE 2, ELAND, MAQ, PROBEMATCH, SOAP, BWA or SEQMAP, or variations thereof or combinations thereof. The terms “aligned,” “alignment,” or “aligning” generally refer to two or more nucleic acid sequences that can be identified as a match (e.g., 100% identity) or partial match. Alignments can be done manually or by a computer (e.g., a software, program, subsystem, or algorithm), non-limiting examples of which include the Efficient Local Alignment of Nucleotide Data (ELAND) computer program distributed as part of the Illumina Genomics Analysis pipeline. Alignment of a sequence read can be a 100% sequence match. In some cases, an alignment is less than a 100% sequence match (i.e., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 or 5 mismatches. Two or more sequences can be aligned using either strand (e.g., sense or antisense strand). In certain embodiments a nucleic acid sequence is aligned with the reverse complement of another nucleic acid sequence. The results from alignment are deposited in an alignment file (e.g., BAM).

As a quality control step, all alignment files may be filtered to remove non-primary alignment records, reads mapped to improper pairs, and reads with more than six edits. Individual bases are excluded if their Phred base quality is less than 30 in tumor samples and less than 20 in normal samples. As described herein, the term “less than” comprises all whole numbers and rational numbers. For example, less than 30 includes 29.9, 29.8, 29.7, 29.6, 29.5, 29.4, 29.3, 29.2, 29.1, 29.0, 25, 20, 15, 10, 5, and 0.

During reference genome alignment 370, variations between the sample and the reference genome may be identified. The process of comparing sequence data to a reference is called variant calling 375. As described herein, variants comprise naturally occurring alterations to a DNA sequence not found in the reference sequence, and the alterations can be classified as benign, likely benign, variant of unknown significance, likely pathogenic or pathogenic. Moreover, variants can comprise both germline variants (e.g., variants present in all the body's cells) and somatic variants (variants that arise during the lifetime of an individual, such as if an individual develops cancer). Examples of variants include small sequence variants (less than 50 base pairs) such as single nucleotide variants (SNVs), single nucleotide polymorphisms (SNPs) and small structural variants (SVs) (e.g., deletions, insertions, insertions and deletions, sometimes referred to as indels) and larger (greater than 50 base pairs) SVs such as chromosomal rearrangements (e.g., translocations and inversions) and copy number changes. SNVs/SNPs are the result of single point mutations that can cause synonymous changes (nucleotide change does not alter the encoded amino acid), missense changes (nucleotide change does alter the encoded amino acid), or nonsense changes (resulting amino acid change converts the encoded codon to a stop codon). Further, variants can occur in both coding and non-coding regions of the genome and can be detected by WGS, as opposed to targeted gene panels, and target specific probes.

Variant calling 375 uses one or more variant calling tools to examine the aligned/mapped sequencing data and reference genome side-by-side to determine the existence of sequence mutations (single base changes and small indels). The variant calling tool may extract candidate variants from alignment data, score a number of individual metrics for each variant, and apply these scores both individually and in combination to identify bona fide sequence mutations and to exclude sequence artifacts. In some embodiments, at least one, or more, substitutions, small indels, and larger alterations such as rearrangements, copy number variation, and microsatellite instability can be determined from the sequencing data. Any suitable technique/variant calling tool may be used to detect structural alterations such as, for example, MuTect, Strelka, and/or JointSNVMix2.

The list of detected variants and their properties (e.g., type of variant) are annotated and deposited in a variant file (e.g., variant call format (VCF)). The output VCF files from variant calling 375 may be accessed by ctDNA algorithm 390 or by a machine learning pipeline (described in section III) to determine variant scores (e.g., importance scores). A VCF file for a single sample can include about 1,500 to about 800,000 variants; however, more or less variants may be found depending on the sample.

### Identifying and Filtering Tumor-Specific Somatic Alterations to Generate Candidate Alterations

Any suitable method, like the aforementioned methods above, may be used to compare tumor sequencing data and normal sequencing data to a reference human genome to identify somatic alterations and their associated features (e.g., coverage, mutant allele fraction, quality score, confidence score). Suitable reference human genomes may include a published human genome (e.g., hgl8 or hg36), sequence data from sequencing a related sample (e.g., a patient's nontumor DNA), or some other reference material, such as “gold standard” sequences obtained by, e.g., Sanger sequencing of subject nucleic acid. The variant calling analysis (e.g., patient sample to reference human genome) may identify a variety of chromosomal alterations (e.g., rearrangements or amplifications), genomic signatures (e.g., microsatellite instabilities), as well as sequence mutations (single base substitutions and small indels).

The tumor identified variants and the normal (germline) variants may be filtered using a set of criteria. The filtering criteria can include removing: (i) variants annotated as low confidence, (ii) variants annotated as indels, (iii) variants observed in genomic databases (e.g., 1000 Genomes or gnomAD germline databases), (iv) variants overlapping simple tandem repeats (e.g., the UCSC simple tandem repeats track), (v) variants with positions with less than 10× coverage, (vi) variants with positions with an alternate allele count less than 4 in the tumor or greater than 1 in the normal, (vii) variants with a variant allele frequency less than 0.05, or any combination thereof.

Additionally or alternatively, stricter filtering criteria may be applied to variants with cytosines substituted for thymines, or guanines substituted for adenines, which may be associated with pre-analytical technical artifacts. Variants with these substitution patterns are removed if the variant allele frequency is less than 0.20 or the alternate allele count is less than 10. The final filtered tumor variants and their properties as well as the normal (germline) variants and their properties are stored in VCF files.

To identify high confidence, whole genome tumor-specific somatic alterations 380, any germline mutations that may be present in the tumor variant VCF file are removed. This is achieved by comparing patient tumor identified variants to their non-tumor reference (e.g., sequence data from the same patient's normal/germline DNA). Germline mutations, mutations present in every cell of the patient, are considered background noise or false positive tumor mutations. If the tumor sequencing data were only compared to a reference human genome, the resulting VCF file would include both somatic and germline mutations. By filtering out the germline mutations, the candidate somatic variant calls are significantly more likely to be indicative of the patient's tumor somatic mutation profile. Such a profile cannot be achieved by performing WGS only on tumor samples. Nor can a purified, high confident, whole genome tumor-specific profile be obtained from gene panels or targeted probes.

Candidate somatic variant calls are compared to a set of reference noncancerous plasma donors. If a candidate somatic variant was present in at least 10% of noncancerous donors or any one of the noncancerous donors contained the variant with at least 25% variant allele frequency, the variant was filtered out. The number of candidate somatic variant calls can include about 1,500 to about 800,000 variants; however, more or less candidate somatic variant calls may be identified based on the samples. This step can be performed separately or external to the machine learning model. Alternatively, this step can be configured into the machine learning model so that the threshold can be fine-tuned by training the machine learning model.

Identifying and Filtering ctDNA Alterations to Determine Candidate Alterations

Variant calling 375 also generates whole genome cfDNA variants 385 from the patients' non-tissue sequencing data files. Initially the non-tissue cfDNA sequencing data files may be compared to a reference human genome to identify whole genome cfDNA variants 385. The unfiltered whole genome cfDNA variants 385 may be compared to the list of filtered candidate somatic variant calls, and only the candidate somatic alterations found in both the cfDNA variant list, and the candidate somatic list may be selected to generate a final list of candidate somatic variant calls specific to the patient's MRD tumor profile. The final list of candidate somatic variants can include about 40,000 to about 70,000 variants; however, more or less candidate somatic variant calls may be identified based on the samples.

The final list of candidate somatic variant calls may be input into a ctDNA algorithm 390 (e.g., ctDNA predictor 250 described with respect to FIG. 2) to predict if the patient's non-tissue sample is ctDNA+ or ctDNA−. The ctDNA status can also be used to estimate the level of ctDNA present 393. In some embodiments, the ctDNA algorithm 390 includes a pretrained machine learning model (MLM) that filters the final list of candidate somatic variant calls and generates a variant score for each of the candidate somatic variant calls. The variant score for each candidate somatic variant is between 0-1 (inclusive) and is determined using the set of features corresponding to each of the candidate somatic variant calls. Features refer to all manner of quality features output from sequencing, alignment, variant calling, or any combination thereof. For example, features may include metrics from the FASTQ files such as quality scores for any given base in the sequence data, quality of alignment, quality of reads, strand information, and metrics relating to the complexity of the region in the genome (e.g., repeat regions and other regions prone to NGS sequencing error). Regarding variant calling, features may include a confidence or probability score output by the variant caller when a variant is identified and/or the quality of the base of the variant. Variants with a score greater than a predetermined threshold (e.g., 0.25), distinct overlap mutants greater than zero, tumor mutant reads mapping quality scores greater than or equal to 30, mutant mismatch average less than or equal to five, and any numerical value for average fragment size are retained. The pretrained MLM may also generate variant scores for a reference cohort of variants that are identified from noncancerous samples using the same method just described.

Once each candidate somatic variant call is given a variant score, all the variant scores for the non-tissue sample are summed and divided by the total number of candidate somatic variants to give a normalized variant score. The normalized variant score may be used as the primary measure for detection of cancer (e.g., whether the non-tissue sample is ctDNA+ or ctDNA−). A non-tissue sample is considered ctDNA+ when the normalized variant score is greater than or equal to the maximum normalized variant score plus one standard deviation of the reference cohort variants.

A ctDNA level for the non-tissue sample is determined by taking the total number of distinct overlapping variant reads, where the variant has a scores greater than 0.25, over the sum of (1) distinct overlapping reads per observed variant and (2) the product of the median genome wide distinct overlapping read coverage with the total unobserved candidate somatic variants to give an estimated ctDNA fraction (as a percent). In other words, the estimated ctDNA level represents a proportion of the total cfDNA collected from the patient.

As a quality control check, ctDNA algorithm 390 can also perform a SNP quality control check 396 to confirm that the datasets obtained from the tumor, normal, and non-tissue samples are derived from the same patient based on the detected SNPs and their associated allele fractions. This step ensures that a sample swap did not occur at any point in the preparation or analysis of the sample set. An SNP quality control (QC) report 399 may be generated and an exemplary summary of the quality control metrics for SNP check that may appear in the SNP QC report 399 are provided in Table 1.

TABLE 1

| | Number of | Number of | | | | Median |
|---|---|---|---|---|---|---|
| | Replicates | Replicates Passing | | | Median | Plasma |
| Study | per Study | SNP check | | Threshold | MutPct_Tumor | MutPct |
| LoB | 24 | Not Evaluated | | 0.8 | N/A | N/A |
| LoD | 27 | 100% | (27/27) | | 0.98 | 0.98 |
| Accuracy | 136 | 100% | (136/136) | | 0.97 | 0.97 |
| DNA Input | 27 | 0% | (0/27) | | 0.96 | 0.55* |
| External | 30 | 100% | (30/30) | | 0.88 | 0.88 |
| Control | | | | | | |

*Indicates plasma samples for DNA input study do not match tumor/normal DNA.

Table 1 shows the quality control metrics for SNP checks for a limit of blank (LoB) study, a limit of detection (LoD) study, an accuracy/clinical confirmation study, and for external controls. The objective of a LoB study is to determine the highest apparent concentration of ctDNA expected to be found when replicates of a sample containing no ctDNA (e.g., normal, noncancerous tissue, buffy coated blood fraction, and the like) are tested. The objective of a LoD study is to determine the lowest concentration of ctDNA likely to be reliably distinguished from a LoB study. In other words, LoD determines the lowest feasible concentration at which ctDNA may be detected in a contrived tumor sample (e.g., synthetically generated) at various concentrations. As shown, 100% of replicates passed the SNP check at a threshold of 0.8, indicating that SNPs could be accurately identified with a median of 0.98 MutPct (e.g., variant allele frequency) for both tumor and plasma. The objective of the accuracy/clinical confirmation study is to determine the analytical accuracy (e.g., the closeness of agreement between the true result and a test result) of ctDNA to be detected by assessing concordance of sequencing and variant calling with an orthogonal test. As shown, 100% of replicates passed the SNP check at a threshold of 0.8, with a median of 0.97 MutPct for both tumor and plasma. The objective of a DNA input guard banding study is to determine the range at which the DNA input amount can vary from the recommended input amount and still produce accurate results. In some cases, the range may be ±20% of the recommended input amount. At 0.8 threshold, 0% of the DNA input studies passed SNP QC check indicating that these samples were not derived from the same patient.

The raw sequencing files (e.g., FASTQ files), processed sequencing files (e.g., alignment/mapping files), and variant calling files generated from the sample processing and computational workflow 300 may be stored in a storage device, such as a server, a database, or a data repository like the ones described in FIG. 2. The files may be stored locally, remotely, and/or on a cloud server. Each file may be stored in association with an identifier of a subject and a date (e.g., a date when a sample was collected and/or a date when the file was generated). During analysis, one or more files may further be transmitted to another system (e.g., a machine learning pipeline or deployment system, as described in further detail herein).

### IV. TRAINING AND IMPLEMENTATION OF A MACHINE LEARNING MODEL

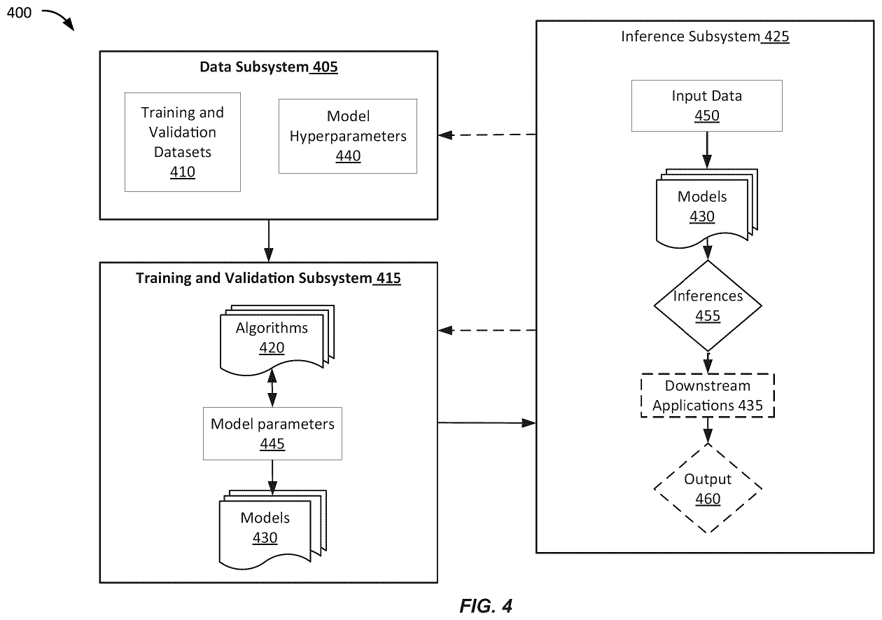

FIG. 4 shows a block diagram of an exemplary machine learning pipeline 400 comprising several subsystems that work together to train, validate, and implement one or more machine learning models in accordance with various embodiments. The machine learning pipeline 400 may be executed as part of the ctDNA predictor 250 or prognosis predictor 255 of the MRD detector platform 215 described in FIG. 2. The machine learning pipeline 400 comprises a data subsystem 405 for collecting, generating, preprocessing, and labeling of training and validation datasets 410, and collecting, generating, setting, or implementing model hyperparameters 440, a training and validation subsystem 415 that facilitates the training and validation of one or more machine learning algorithms 420 and the generation of one or more machine learning models 430, and an inference subsystem 425 for deploying and implementing the one or more trained machine learning models 430 independently or in combination with one or more downstream applications 435 for further processes (e.g., providing diagnosis or administering a treatment).

As used herein, machine learning algorithms (also described herein as simply algorithm or algorithms) are procedures that are run on datasets (e.g., training and validation datasets) and extract features from the datasets, perform pattern recognition on the datasets, learn from the datasets, and/or are fit on the datasets. Examples of machine learning algorithms include linear and logistic regressions, decision trees, random forest, support vector machines, principal component analysis, Apriori algorithms, gradient descent algorithms, Hidden Markov Model, artificial neural networks, k-means clustering, and k-nearest neighbors. As used herein, machine learning models (also described herein as simply model or models) are the output of the machine learning algorithms and are comprised of model parameters and prediction algorithm(s). In other words, the machine learning model is the program that is saved after running a machine learning algorithm on training data and represents the rules, numbers, and any other algorithm-specific data structures required to make inferences. For example, a linear regression algorithm may result in a model comprised of a vector of coefficients with specific values, a decision tree algorithm may result in a model comprised of a tree of if-then statements with specific values, a random forest algorithm may result in a random forest model that is an ensemble of decision trees for classification or regression, or neural network, backpropagation, and gradient descent algorithms together result in a model comprised of a graph structure with vectors or matrices of weights with specific values.

### A. Data Subsystem 405

Data subsystem 405 is used to collect, generate, preprocess, and label data to be used by the training and validation subsystem 415 to train and validate one or more machine learning algorithms 420. The data subsystem 405 comprises training and validation datasets 410 and model hyperparameters 440. Raw data may be acquired through a public database or a commercial database. For example, the data subsystem 405 may access and load paired sequencing data and variant data from data repositories, such as data repositories 210 described in FIG. 2. The paired sequencing data and variant data may be generated by performing WGS and analysis from biological samples obtained from the same patient. The paired sequencing and variant data accessed by data subsystem 405 can include a set of sequences or sequence reads that include mutations and/or structural alterations. The data subsystem 405 may also access WGS and variant files for a set of longitudinal samples collected from the same patient over a treatment plan. The acquired raw data may be further preprocessed to generate the training and validation datasets 410.

Preprocessing may be implemented by the data subsystem 405, serving as a bridge between raw data acquisition and effective model training. The primary objective of preprocessing is to transform raw data into a format that is more suitable and efficient for analysis, ensuring that the data fed into machine learning algorithms is clean, consistent, and relevant. This step can be useful because raw data often comes with a variety of issues such as missing values, noise, irrelevant information, and inconsistencies that can significantly hinder the performance of a model. By standardizing and cleaning the data beforehand, preprocessing helps in enhancing the accuracy and efficiency of the subsequent analysis, making the data more representative of the underlying problem the model aims to solve.

Raw data preprocessing may comprise data synthesis and/or data augmentation. Different data synthesis and/or data augmentation techniques may be implemented by the data subsystem 405 to generate pre-processed data to be used for the training and validation subsystem 415. Data synthesizing involves creating entirely new data points from scratch. This technique may be used when real data is insufficient, too sensitive to use, or when the cost and logistical barriers to obtaining more real data are too high. The synthesized data should be realistic enough to effectively train a machine learning model, but distinct enough to comply with regulations (e.g., privacy regulations (such as the Health Insurance Portability and Accountability Act in the United States) and ethical guidelines), if necessary. Techniques such as Generative Adversarial Networks (GANs) or Variational Autoencoders (VAEs) may be used to generate new data examples. These models learn the distribution of real data and attempt to produce new data examples that are statistically similar but not identical. Data augmentation, on the other hand, refers to techniques used to artificially expand the size of a dataset by creating modified versions of existing data examples. The primary goal of data augmentation is to increase variation in the data in order to make the model more robust to variations it might encounter in the real world, thereby improving its ability to generalize from the training data to unseen data.

Other raw data preprocessing techniques include data cleaning, normalization, feature extraction, dimensionality reduction, and the like. Data cleaning may involve removing duplicates, filling in missing values, or filtering out outliers to improve data quality. Normalization involves scaling numeric values to a common scale without distorting differences in the ranges of values, which helps prevent biases in the model due to the inherent scale of features. Feature extraction involves transforming the input data into a set of useable features, possibly reducing the dimensionality of the data in the process. For instance, raw sequencing data might comprise the initial output generated by sequencing machines from a sequencing assay. This initial output is typically in the form of raw sequence reads, which are short nucleotide sequences (e.g., DNA or RNA) that represent fragments of the genome or transcriptome being sequenced. Feature extraction may transform the raw sequencing data into a set of features including coverage, mutant allele fraction, quality scores, and/or confidence scores. For example, a WGS and analysis assay produce a variety of different sequencing, alignment, mapping, variant calling, quality control files, and the like that each include all types of features that describe characteristics or properties of the sequencing, alignment/mapping, variant calling, and quality control files. Sequencing features extracted may include metrics from FASTQ files such as quality scores for any given base in the sequence data, quality of alignment, quality of reads, and metrics relating to the complexity of the region in the genome (e.g., repeat regions and other regions prone to NGS sequencing error). Variant calling features may also be extracted, including a confidence or probability score that is output by the variant caller when a variant is identified and/or the quality of the base of the variant. The number of features depends on the project's need, for example, about 10 features to about 500 features may be extracted. In some instances, the extracted features include at least 62 predetermined features. It should be understood that more or less features may be considered.

Dimensionality reduction techniques like Principal Component Analysis (PCA) may be used to reduce the number of variables under consideration, by obtaining a set of principal variables. These techniques not only help in reducing the computational load on the model but also in mitigating issues like overfitting by simplifying the data without losing critical information.

In the instance that machine learning pipeline 400 is used for supervised or semi-supervised learning of machine learning models, labeling techniques can be implemented as part of the data preprocessing. The quality and accuracy of data labeling directly influence the model's performance, as labels serve as the definitive guide that the model uses to learn the relationships between the input features and the desired output. Particularly in complex domains such as cancer detection and medical diagnosis, precise and consistent labeling is important because it provides the ground truth or target outcomes against which the model's predictions are compared and adjusted during training. Effective labeling ensures that the model is trained on correct and clear examples, thus enhancing its ability to generalize from the training data to real-world scenarios. In some instances, the ground truth value is provided within the raw data.

In some instances, the ground truth values (labels) are provided within the raw data. For example, when the raw data includes sequencing data, the labels may include variant types. Many different variant types may be included in the variant files accessed and loaded by the data subsystem 405. For example, the variants may include benign, likely benign, variant of unknown significance, likely pathogenic or pathogenic variants. The variants may comprise germline variants, somatic variants, or a combination thereof. Different structural variants may be included such as small structural variants (less than 50 base pairs) such as single nucleotide variants (SNVs), single nucleotide polymorphisms (SNPs) and small structural sequence variants (SVs) (e.g., deletions, insertions, insertions and deletions, sometimes referred to as indels) and larger (e.g., greater than 50 base pairs) SVs such as chromosomal rearrangements (e.g., translocations and inversions). In some instances, the variant types may be substitutions, small indels, and larger alterations such as rearrangements, copy number variation, and microsatellite instabilities.

Labeling techniques can vary significantly depending on the type of data and the specific requirements of the project. Manual labeling, where human annotators label the data, is one method that can be used. This approach may be useful when a detailed understanding and judgment are required, such as in labeling medical data or categorizing text data where context and subtlety are important. However, manual labeling can be time-consuming and prone to inconsistency, especially with a large number of annotators. To mitigate this, semi-automated labeling tools may be used as part of data subsystem 405 to pre-label data using algorithms, which human annotators may then review and correct as needed. Another approach is active learning, a technique where the model being developed is used to label new data iteratively. The model suggests labels for new data points, and human annotators may review and adjust certain predictions such as the most uncertain predictions. This technique optimizes the labeling effort by focusing human resources on a subset of the data, e.g., the most ambiguous cases, improving efficiency and label quality through continuous refinement.

For example, when the raw data includes sequencing data, the labels may include whether a variant is a true positive mutation or a false positive mutation. True positive mutations/variants can be obtained from clinical FFPE tissues, cell lines, plasma cases from patients with cancer or patients with a recurrence after a cancer treatment, or any combination thereof. False positive mutations/variants can be obtained from noncancerous normal FFPE tissues, cells, plasma cases from noncancerous samples or patients without a recurrence after a cancer treatment, or any combination thereof. When a variant is partial-labeled or left unlabeled, a user may update the label of the variant or make an annotation to indicate what portion of the input data should be labeled.

The training and validation datasets 410 may comprise the raw data and/or the preprocessed data. The training and validation datasets 410 are typically split into at least three subsets of data: training, validation, and testing. The training subset is used to fit the model, where the model is configured to make inferences based on the training data. The validation subset, on the other hand, is utilized to tune hyperparameters and prevent overfitting to the training data. Finally, the testing subset serves as a new and unseen dataset for the model, used to simulate real-world applications and evaluate the final model's performance. The process of splitting ensures that the model can perform well not just on the data it was trained on, but also on new, unseen data, thereby validating and testing its ability to generalize.

Various techniques can be employed to split the data effectively, aiming to maintain a good representation of the overall dataset in each subset. A simple random split (e.g., a 70/20/10%, 80/10/10%, or 60/25/15%) is the most straightforward approach, where examples from the data are randomly assigned to each of the three sets. However, more sophisticated techniques may be necessary to preserve the underlying distribution of data. For instance, stratified sampling may be used to ensure that each split reflects the overall distribution of a specific variable, particularly useful in cases where certain categories or outcomes are underrepresented. Another technique, k-fold cross-validation, involves rotating the validation set across different subsets of the data, maximizing the use of available data for training while still holding out portions for validation. These techniques help in achieving more robust and reliable model evaluation and are useful in the development of predictive models that perform consistently across datasets.

Data subsystem 405 can also be used for collecting, generating, setting, or implementing model hyperparameters 440 for the training and validation subsystem 415. The hyperparameters control the overall behavior of the models. Unlike model parameters 445 that are learned automatically during training, model hyperparameters 440 are settings that are external to the model and must be determined before training begins. Model hyperparameters 440 can have a significant impact on the performance of the model. For example, in a neural network, model hyperparameters 440 include the learning rate, number of layers, number of neurons per layer, and/or activation functions, among others, in a random forest, model hyperparameters 440 may include the number of decision trees in the forest, the maximum depth of each decision tree, the minimum number of samples required to be at each leaf node, the maximum number of features to consider when looking for a best split, and/or bootstrap parameters. These settings can determine how quickly a model learns, its capacity to generalize from training data to unseen data, and its overall complexity. Correctly setting hyperparameters is important because inappropriate values can lead to models that underfit or overfit the data. Underfitting occurs when a model is too simple to learn the underlying pattern of the data, and overfitting happens when a model is too complex, learning the noise in the training data as if it were signal. Many different variant types may be included in the variant files accessed and loaded by data generator 405. For example, the variants may include benign, likely benign, variant of unknown significance, likely pathogenic or pathogenic variants. The variants may comprise germline variants, somatic variants, or a combination thereof. Different structural variants may be included such as small structural variants (less than 50 base pairs) such as single nucleotide variants (SNVs), single nucleotide polymorphisms (SNPs) and small structural sequence variants (SVs) (e.g., deletions, insertions, insertions and deletions, sometimes referred to as indels) and larger (greater than 50 base pairs) SVs such as chromosomal rearrangements (e.g., translocations and inversions). In some embodiments the variants may be substitutions, small indels, and larger alterations such as rearrangements, copy number variation, and microsatellite instabilities.

### B. Training and Validation Subsystem 415

The training and validation subsystem 415 is comprised of a combination of specialized hardware and software to efficiently handle the computational demands required for training, validating, and testing machine learning algorithm/model. On the hardware side, high-performance GPUs (Graphics Processing Units) may be used for their ability to perform parallel processing, drastically speeding up the training of complex models, especially deep learning networks. CPUs (Central Processing Units), while generally slower for this task, may also be used for less complex model training or when parallel processing is less critical. TPUs (Tensor Processing Units), designed specifically for tensor calculations, provide another level of optimization for machine learning tasks. In some instances, a Field-Programmable Gate Array (FPGA), or a specifically designed FPGA may be used to perform the training, validating, and/or testing tasks,

Training is the initial phase of developing machine learning models 430 where the model learns to make predictions, classifications, or decisions based on training data provided from the training and validation datasets 410. During this phase, the model iteratively adjusts its internal model parameters 445 to achieve a preset optimization condition. In a supervised machine learning training process, the preset optimization condition can be achieved by minimizing the difference between the model output (e.g., predictions, classifications, or decisions) and the ground truth labels in the training data. In some instances, the preset optimization condition can be achieved when the preset fixed number of iterations or epochs (full passes through the training dataset) is reached. In some instances, the preset optimization condition is achieved when the performance on the validation dataset stops improving or starts to degrade. In some instances, the preset optimization condition is achieved when a convergence criterion is met, such as when the change in the model parameters falls below a certain threshold between iterations. This process, known as fitting, is fundamental because it directly influences the accuracy and effectiveness of the model.

In an exemplary training phase performed by the training and validation subsystem 415, the training subset of data is input into the machine learning algorithms 420 to find a set of model parameters 445 (e.g., weights, coefficients, trees, feature importance, and/or biases) that minimizes or maximizes an objective function (e.g., a loss function, a cost function, a contrastive loss function, a cross-entropy loss function, an Out-of-Bag (OOB) score, etc.). To train the machine learning algorithms 420 to achieve accurate predictions, “errors” (e.g., a difference between a predicted label and the ground truth label) need to be minimized. In order to minimize the errors, the model parameters can be configured to be incrementally updated by minimizing the objective function over the training phase (“optimization”). Various different techniques may be used to perform the optimization. For example, to train machine learning algorithms such as a neural network, optimization can be done using back propagation. The current error is typically propagated backwards to a previous layer, where it is used to modify the weights and bias in such a way that the error is minimized. The weights are modified using the optimization function. Other techniques such as random feedback, Direct Feedback Alignment (DFA), Indirect Feedback Alignment (IFA), Hebbian learning, and the like can also be used to update the model parameters 445 in a manner as to minimize or maximize an objective function. This cycle is repeated until a desired state (e.g., a predetermined minimum value of the objective function) is reached.

The training phase is driven by three primary components: the model architecture (which defines the structure of the algorithm(s) 420), the training data (which provides the examples from which to learn), and the learning algorithm (which dictates how the model adjusts its model parameters). The goal is for the model to capture the underlying patterns of the data without memorizing specific examples, thus enabling it to perform well on new, unseen data.

The model architecture is the specific arrangement and structure of the various components and/or layers that make up a model. In the context of a neural network, the model architecture may include the configuration of layers in the neural network, such as the number of layers, the type of layers (e.g., convolutional, recurrent, fully connected), the number of neurons in each layer, and the connections between these layers. In the context of a random forest consisting of a collection of decision trees, the model architecture may include the configuration of features used by the decision trees, the voting scheme, and hyperparameters such as the number of trees in the forest, the maximum depth of each tree, the minimum number of samples required to split a node, and the maximum number of features to consider when looking for the best split. In some instances, the model architecture is configured to perform multiple tasks. For example, a first component of the model architecture may be configured to perform a feature selection function, and a second component of the model architecture may be configured to perform a feature scoring function. The different components may correspond to different algorithms or models, and the model architecture may be an ensemble of multiple components.

Model architecture also encompasses the choice and arrangement of features and algorithms used in various models, such as decision trees or linear regression. The architecture determines how input data is processed and transformed through various computational steps to produce the output. The model architecture directly influences the model's ability to learn from the data effectively and efficiently, and it impacts how well the model performs tasks such as classification, regression, or prediction, adapting to the specific complexities and nuances of the data it is designed to handle.

The model architecture can encompass a wide range of algorithms 420, suitable for different kinds of tasks and data types. Examples of algorithms 420 include, without limitation, linear regression, logistic regression, decision tree, Support Vector Machines, Naives Bayes algorithm, Bayesian classifier, linear classifier, K-Nearest Neighbors, K-Means, random forest, dimensionality reduction algorithms, grid search algorithm, genetic algorithm, AdaBoosting algorithm, Gradient Boosting Machines, and Artificial Neural Networks such as convolutional neural network (“CNN”), an inception neural network, a U-Net, a V-Net, a residual neural network (“Resnet”), a transform neural network, a recurrent neural network, a Generative adversarial network (GAN), or other variants of Deep Neural Networks (“DNN”) (e.g., a multi-label n-binary DNN classifier or multi-class DNN classifier). These algorithms can be implemented using various machine learning libraries and frameworks such as TensorFlow, PyTorch, Keras, and scikit-learn, which provide extensive tools and features to facilitate model building, training, validation, and testing. For example, the ctDNA algorithm 390 described with respect to FIG. 3 could be a random forest algorithm. In some instances, the ctDNA algorithm 390 may be a combination of different algorithms, e.g., a combination of a grid search algorithm and a random forest algorithm.

The learning algorithm is the overall method or procedure used to adjust the model parameters 445 to fit the data. It dictates how the model learns from the data provided during training. This includes the steps or rules that the algorithm follows to process input data and adjust the model's internal parameters (e.g., weights in neural networks) based on the output of the objective function. Examples of learning algorithms include gradient descent, backpropagation for neural networks, and splitting criteria in decision trees.

Various techniques may be employed by training and validation subsystem 415 to train machine learning models 430 using the learning algorithm, depending on the type of model and the specific task. For supervised learning models, where the training data includes both inputs and expected outputs (e.g., ground truth labels), gradient descent is a possible method. This technique iteratively adjusts the model parameters 445 to minimize or maximize an objective function (e.g., a loss function, a cost function, a contrastive loss function, etc.). The objective function is a method to measure how well the model's predictions match the actual labels or outcomes in the training data. It quantifies the error between predicted values and true values and presents this error as a single real number. The goal of training is to minimize this error, indicating that the model's predictions are, on average, close to the true data. Common examples of loss functions include mean squared error for regression tasks and cross-entropy loss for classification tasks.

The adjustment of the model parameters 445 is performed by the optimization function or algorithm, which refers to the specific method used to minimize (or maximize) the objective function. The optimization function is the engine behind the learning algorithm, guiding how the model parameters 445 are adjusted during training. It determines the strategy to use when searching for the best weights that minimize (or maximize) the objective function. Gradient descent is a primary example of an optimization algorithm, including its variants like stochastic gradient descent (SGD), mini-batch gradient descent, and advanced versions like Adam or RMSprop, which provide different ways to adjust learning rates or take advantage of the momentum of changes. For example, in training a neural network, backpropagation may be used with gradient descent to update the weights of the network based on the error rate obtained in the previous epoch (cycle through the full training dataset). Another technique in supervised learning is the use of decision trees, where a tree-like model of decisions is built by splitting the training dataset into subsets based on an attribute value test. This process is repeated on each derived subset in a recursive manner called recursive partitioning. In training a random forest, the set of decision trees can be trained collectively to minimize a Gini impurity or entropy, leading to accurate classification.

In unsupervised learning, where training data does not include labels, different techniques are used. Clustering is one method where data is grouped into clusters that maximize the similarities of data within the same cluster and maximize the differences with data in other clusters. The K-Means algorithm, for example, assigns each data point to the nearest cluster by minimizing the sum of distances between data points and their respective cluster centroids. Another technique, Principal Component Analysis (PCA), involves reducing the dimensionality of data by transforming it into a new set of variables, the principal components, which are uncorrelated and ordered so that the first few retain most of the variation present in all of the original variables. These techniques help uncover hidden structures or patterns in the data, which can be essential for feature reduction, anomaly detection, or preparing data for further supervised learning tasks.

Validating is another phase of developing machine learning models 430 where the model is checked for deficiencies in performance and the hyperparameters 440 are optimized based on validation data provided from the training and validation datasets 410. The validation data helps to evaluate the model's performance, such as accuracy, precision, or recall, to gauge how well the model is likely to perform in real-world scenarios. Hyperparameter optimization, on the other hand, involves adjusting the settings that govern the model's learning process (e.g., learning rate, number of layers, size of the layers in neural networks) to find the combination that yields the best performance on the validation data. One optimization technique is grid search, where a set of predefined hyperparameter values are systematically evaluated. The model is trained with each combination of these values, and the combination that produces the best performance on the validation set is chosen. Although thorough, grid search can be computationally expensive and impractical when the hyperparameter space is large. A more efficient alternative optimization technique is random search, which samples hyperparameter combinations from a defined distribution randomly. This approach can in some instances find a good combination of hyperparameter values faster than grid search. Advanced methods like Bayesian optimization, genetic algorithms, and gradient-based optimization may also be used to find optimal hyperparameters more effectively. These techniques model the hyperparameter space and use statistical methods to intelligently explore the space, seeking hyperparameters that yield improvements in model performance.

An exemplary validation process includes iterative operations of inputting the validation subset of data into the trained algorithm(s) using a validation technique such as K-Fold Cross-Validation, Leave-one-out Cross-Validation, Leave-one-group-out Cross-Validation, Nested Cross-Validation, or the like, to fine tune the hyperparameters and ultimately find the optimal set of hyperparameters. In some instances, a 5-fold cross-validation technique may be used to avoid overfitting the trained algorithm and/or to limit the number of selected features per split to the square-root of the total number of input features. In some instances, training dataset is split into 5 equal-size cohorts (or about equal-size), and every four of the cohorts are used to train an algorithm to generate five models (e.g, cohorts #1, 2, 3, and 4 are used to train and generate model 1, cohorts #1, 2, 3, and 5 are used to train and generate model 2, cohorts #1, 2, 4, and 5 are used to train and generate model 3, cohorts #1, 3, 4, and 5 are used to train and generate model 4, and cohorts #2, 3, 4 and 5 are used to train and generate model 5). Each model is evaluated (or validated) using the unused cohort in the training (e.g., for model 5, cohort #1 is used for validation). The overall performance of the training can be evaluated by an average performance of the five models. K-fold cross-validation provides a more robust estimate of a model's performance compared to a single training/validation split because it utilizes the entire dataset for both training and evaluation and reduces the variance in the performance estimate.

Once a machine learning model has been trained and validated, it undergoes a final evaluation using testing data provided from the training and validation datasets 410, which is a separate subset of the training and validation datasets 410 that generally has not been used during the training or validation phases. This step is crucial as it provides an unbiased assessment of the model's performance in simulating real-world operation. The test dataset serves as new, unseen data for the model, mimicking how the model would perform when deployed in actual use. During testing, the model's predictions are compared against the true values in the test dataset using various performance metrics such as accuracy, precision, recall, and mean squared error, depending on the nature of the problem (classification or regression). This process helps to verify the generalizability of the model its ability to perform well across different data samples and environments highlighting potential issues like overfitting or underfitting and ensuring that the model is robust and reliable for practical applications. The machine learning models 430 are fully validated and tested once the output predictions have been deemed acceptable by user defined acceptance parameters. Acceptance parameters may be determined using correlation techniques such as Bland-Altman method and the Spearman's rank correlation coefficients and calculating performance metrics such as the error, accuracy, precision, recall, receiver operating characteristic curve (ROC), and the like.

### C. Inference Subsystem 425

The inference subsystem 425 is comprised of various components for deploying the machine learning models 430 in a production environment. Deploying the machine learning models 430 includes moving the models from a development environment (e.g., the training and validation subsystem 415, where it has been trained, validated, and tested), into a production environment where it can make inferences on real-world data (e.g., input data 450). This step typically starts with the model being saved after training, including its parameters and configuration such as final architecture and hyperparameters.

Once deployed, the model is ready to receive input data 450 and return outputs (e.g., inferences 455). In some instances, the model resides as a component of a larger system or service (e.g., including additional downstream applications 435). In some instances, the models 430 and/or the inferences 455 can be used by the downstream applications 435 to provide further information. For example, the inferences 455 can be used to determine whether a specific treatment should be administered to a patient. The downstream applications can be configured to generate an output 460. In some instances, the output 460 comprises a report including inferences 455 and information generated by the downstream applications 435.

In an exemplary inference subsystem 425, the input data 450 includes sequencing and variant files generated from one or more biological samples from a patient having been diagnosed a disease (e.g., cancer). The input data 450 may further include clinical data for the same patient that provides information on the type/stage of disease, past, current, and/or future treatment plans, whether the patient has had a recurrence of the disease, and any other information pertinent to the patient. In some instances, the input data 450 comprises clinicopathological risk factors that are associated with distinction of patients whether they are at either a very low risk or a very high-risk of developing a recurrence of the cancer within a certain amount of time (e.g., 3 years). The sequencing and variant files may be generated by performing WGS and variant calling on the one or more biological samples collected from the patient by the sample processing and bioinformatic workflow 300 as described with respect to FIG. 3. The one or more biological samples may be a single non-tissue sample (e.g., a plasma sample, or other samples such as sputum, saliva, cerebral spinal fluid, surgical drain fluid, urine, cyst fluid, etc.) obtained from the patient. The one or more biological samples may also include a tumor sample and a noncancerous sample (e.g., leukocytes or buffy coats, or tissue sample from a part that is known or determined to be cancer-free). In some instances, the one or more biological samples may further include a set of reference samples obtained for noncancerous subjects or donors. Multiple rounds of samples may be collected and used as the input data 450. In some instances, the one or more samples may be collected at any timepoint between pre-surgery and 3 years after surgery. For example, the one or more samples may be collected (i) pre-surgery, (ii) about 3 days to about 65 days post-surgery and before receiving a therapeutic treatment, and/or (iii) about every 6 months up to 3 years post-surgery and after receiving a therapeutic treatment. In some instances, a tumor sample may be collected during the time of surgery. In some instances, a noncancerous sample may also be collected during surgery, or from the non-tissue sample collected at a different time point from the time of surgery.

In some instances, the input data 450 may be preprocessed before inputting into the models 430 to achieve a faster model performance. For example, the input data 450 may be preprocessed by the candidate somatic variant generator 245 processor of the MRD detector platform 215 described with respect to FIG. 2. The input data 450 may be also preprocessed by the sample processing and bioinformatic workflow 300 as described with respect to FIG. 3. The preprocessing may reduce the dimensions of the input data 450 and thus save computing time and resources (e.g., requiring less computer memory) in the inference stage to generate the inferences 455.

To manage and maintain its performance, a deployed model may also be continuously monitored to ensure it performs as expected over time. This involves tracking the model's prediction accuracy, response times, and other operational metrics. Additionally, the model may require retraining or updates based on new data or changing conditions. This can be useful because machine learning models can drift over time due to changes in the underlying data they are making predictions on—a phenomenon known as model drift. Therefore, maintaining a machine learning model in a production environment often involves setting up mechanisms for performance monitoring, regular evaluations against new test data, and potentially periodic updates and retraining of the model to ensure it remains effective and accurate in making predictions.

### V. IDENTIFICATION OF PATIENTS WHO BENEFIT FROM ACT

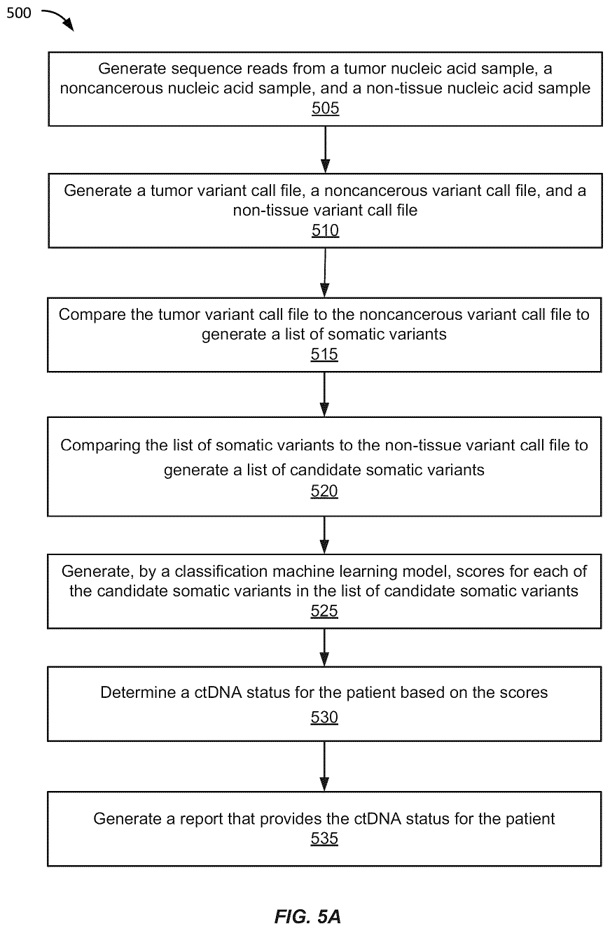

FIG. 5A shows an exemplary workflow 500 for determining the status of a non-tissue sample as ctDNA positive or negative. The processing depicted in FIG. 5A, and also in FIG. 5B, may be implemented in software (e.g., code, instructions, program) executed by one or more processing units (e.g., processors, cores) of the respective systems, hardware, or combinations thereof (e.g., computing environment 200 described with respect to FIG. 2). The software may be stored on a non-transitory storage medium (e.g., on a memory device). The method presented in FIG. 5A and described below is intended to be illustrative and non-limiting. Although FIG. 5A depicts the various processing steps occurring in a particular sequence or order, this is not intended to be limiting.

At block 505, sequence reads from a tumor nucleic acid sample, a noncancerous nucleic acid sample, and a non-tissue nucleic acid sample are generated using whole genome sequencing (WGS). The tumor nucleic acid sample, noncancerous nucleic acid sample, and the non-tissue nucleic acid sample may be obtained from a same patient at a same or different time point. For example, the tumor, noncancerous, and non-tissue samples may be collected at different time points during treatment for the patient, e.g., samples may be collected (i) pre-surgery (ii) during surgery, and (iii) about 3 days to about 65 days post-surgery before receiving a therapeutic treatment (e.g., adjuvant chemotherapy (ACT)). The patient may have previously been diagnosed with a cancer and undergone surgery to remove one or more tumors. The patient has preferably been diagnosed with the cancer can be a colon cancer; however, other cancer types may be considered (e.g., a head and neck cancer, a lung cancer, a breast cancer, a melanoma cancer, or the like etc.). It is can be unknown whether the patient has a low or high-risk of cancer recurrence after surgery and thus, whether a secondary therapeutic option is beneficial. In several embodiments, the preferred secondary therapeutic treatment option is an adjuvant chemotherapy (ACT), however, other secondary therapeutic options may be considered. In some instances, the non-tissue nucleic acid sample comprises cell-free nucleic acid extracted from a plasma sample, and the plasma sample is isolated by adding an anticoagulant to a blood sample and centrifuging the blood sample at sufficient speed to separate the plasma from the blood cells.

In some instances, the non-tissue samples comprise nucleic acids, such as cell free DNA, that are released by cells undergoing apoptosis or necrosis. In addition, the non-tissue sample may also comprise ctDNA in extremely low abundance (e.g., often present at levels of less than 0.10% of total cell free DNA). Depending on when during treatment and/or how the patient responds to surgery, the non-tissue sample may be ctDNA+ or ctDNA−. Optionally, from the non-plasma fraction, noncancerous samples may be acquired, for example as blood cells, white blood cells, the buffy coated fraction, etc. In addition to or instead of, a noncancerous sample may be collected during the time of surgery with the tumor sample. In this instance, the noncancerous sample may be any bodily tissue or fluid containing nucleic acid that is considered to be cancer-free. The tumor sample may be collected during the time of surgery as tissue, cells, plasma, blood, cell free DNA, circulating tumor DNA, or any combination thereof.

At block 510, a tumor variant call file, a noncancerous variant call file, and a non-tissue variant call file are generated. The generation may be performed by analyzing the sequence reads corresponding respectively to the tumor nucleic acid sample, the noncancerous nucleic acid sample, and the non-tissue nucleic acid sample. The analysis can be performed by the sample processing and computational workflow 300 described with respect to FIG. 3. The v Variant call files, or VCF files, comprise a list of all the detected variants, their properties (e.g., variant type), as well as their quality features for a single sample. The initial variant call files are the result of comparing an experimental sample (e.g., the tumor nucleic acid sample, the noncancerous nucleic acid samples, and the plasma samples) to a reference or “gold standard” genome where the difference/variations identified between the experimental sample and the reference are recoded in the corresponding VCF file.

At block 515, the tumor variant call file is compared to the noncancerous variant call file to generate a list of somatic variants. In some instances, variants in the noncancerous variant call file are treated as “germline variants” that do not have an informative effect in determining the true positive mutations of a non-tissue sample. The “germline variants” will be excluded or removed from the tumor variant call file. The remaining variants in the tumor variant call file are the somatic variants.

At block 520, the list of somatic variants is compared to the non-tissue variant call file to generate a list of candidate somatic variants. In some instances, only a variant that appears in both the list of somatic variants and the non-tissue variant call file will be considered as a candidate somatic variant. In some instances, other criteria or variant files may be used to generate the list of candidate somatic variants. The list of candidate somatic variants may comprise substitutions, small indels, chromosomal rearrangements, copy number variation, microsatellite instabilities, or any combination thereof. In addition, the sets of candidate somatic variants also retain information pertaining to their properties (e.g., variant type) as well as their quality features.

In some instances, a SNP quality control check may also be performed to confirm that the datasets obtained from the tumor, noncancerous, and non-tissue samples are derived from the same patient based on the detected SNPs and their associated allele fractions. This step ensures that a sample swap did not occur at any point in the preparation or analysis of the sample set.

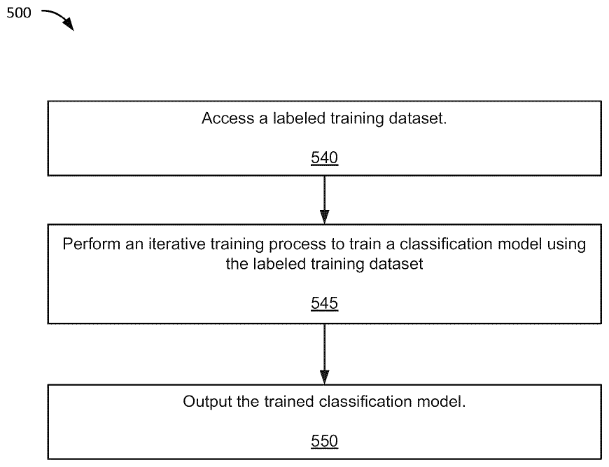

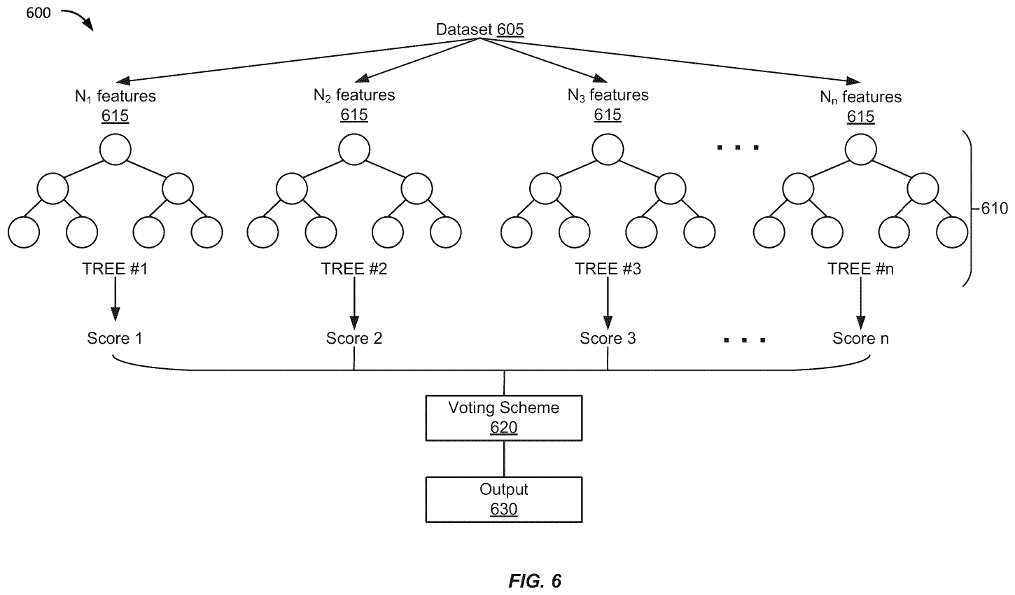

At block 525, scores for each of the candidate somatic variants in the list of candidate somatic variants may be generated using a classification machine learning model. The scores may be generated based on a plurality of classifications generated by the classification machine learning model. In some instances, the scores comprise a variant score for each candidate somatic variant. In various embodiments, the classification machine learning model is a random forest classification model that comprises an ensemble of decision trees (see, e.g., FIG. 6). In some instances, the classification machine learning model is configured to generate the variant scores by filtering the candidate somatic variants through a series of yes/no questions and assigning a variant score (e.g., a confidence/probability score) to each variant. For example, the first candidate somatic variant (e.g., input) along with its corresponding features, are input into the classification model. When the input traverses down tree one corresponding to a first feature, the yes/no question may be, does the value of the input feature meet a threshold value for the first feature. If yes, the input variant may receive a score of 1. This process repeats for each feature for the input variant, until all trees in the ensemble have generated a score. Assuming the random forest comprises 62 trees and if the input variant scored yes (e.g., 1) on 58 of the trees, the final variant score is 58/62=0.94. The final variant score may be compared to a predetermined threshold to determine whether the input variant is removed or not. In some instances, the individual trees making up the forest ensemble may have different relative weights depending how predictive that particular feature is to the overall classification.

In some instances, the classification machine learning model is an ensemble of multiple models that is configured to perform a variant selection before inputting the candidate somatic variants random forest classification model. The variant selection may be performed based on a searching model or a selection model. In some instances, the searching or selection model is also pretrained using a process described with respect to FIG. 4. The candidate somatic variants are searched and a subset of variants is selected using the searching or selection model.

In some instances, the variant scores generated form the classification model can also be used to determine the status (e.g., presence or absence) of ctDNA in the non-tissue sample as well as estimate the level of ctDNA in the non-tissue sample. To determine the status of ctDNA in the non-tissue sample, all the variant scores for the non-tissue sample are summed and divided by the total number of candidate somatic variants to give a normalized variant score. The normalized variant score may be used as the primary measure for detection of cancer (e.g., whether the non-tissue sample is ctDNA+ or ctDNA−). A non-tissue sample is considered ctDNA+ when the normalized variant score is greater than or equal to the maximum normalized variant score plus one standard deviation of the reference cohort variants.

At block 530, a ctDNA status is determined for the non-tissue nucleic acid sample of the patient based on the scores. The ctDNA status can be either positive or negative. The ctDNA status can be determined by taking the total number of distinct overlapping variant reads, where the variant has a scores greater than 0.25, over the sum of (1) distinct overlapping reads per observed variant and (2) the product of the median genome wide distinct overlapping read coverage with the total unobserved candidate somatic variants to give an estimated ctDNA fraction (as a percent). In other words, the estimated ctDNA fraction within the total cfDNA collected from the patient's non-tissue is compared to the ctDNA distribution observed from a reference cohort of healthy (e.g., noncancerous) individuals to determine the positive or negative status.

At block 535, a report is generated to provide the ctDNA status for the patient. In some instances, the report may comprise other information, for example, a configured genome of the patient using the sequence reads, or some or all variants in the tumor variant call file, the noncancerous variant call file, and/or the non-tissue variant call file.

FIG. 5B shows an exemplary workflow for training a classification model, more specifically a random forest classification model.

At block 540, a labeled training dataset is accessed. The labeled training dataset comprises WGS of thousands of ground truth true positive mutations and their associated features from clinical FFPE tissues, cell lines, plasma cases from patient(s) with cancer, or any combination thereof and their corresponding features. In addition, the labeled training dataset can also comprise WGS of thousands of ground truth false positive mutations and their associated features from healthy (e.g., noncancerous) normal FFPE tissues, cells, plasma cases from noncancerous samples, or any combination thereof and their corresponding features are included. The true/false positive mutations may include one or more examples of substitutions, small indels, rearrangements, copy number variation, microsatellite instabilities, or any combination thereof per sample. Further, the sample data can include sequencing results and variant calls generated by diluting samples by different dilution levels to achieve various DNA concentrations and sequencing the diluted samples. For example, biological samples (e.g., tissue samples, noncancerous samples, and/or non-tissue samples) may be diluted at a dilution level of about 0.01, about 0.001, about 5×10⁻⁴, about 2×10⁻⁴, about 1×10⁻⁴, about 5×10⁻⁵, or about 1×10⁻⁵.

### 1. Random Forest Machine Learning Model Training and Implementation

FIG. 6 shows an exemplary illustration of a random forest machine learning model 600 in accordance with various embodiments. For example, the random forest machine learning model 600 may be a classification model implemented within a system, for example as part of the ctDNA predictor 250 of the MRD detector platform 215 described with respect to FIG. 2 and/or as the ctDNA algorithm 390 described with respect to FIG. 3. The random forest machine learning model 600 takes dataset 605 (e.g., variants and their corresponding features) as input, and applies different combinations of features 615 to decision trees 610 to generate scores for each variant. The scores can be later used by a voting scheme 620 of the random forest machine learning model 600 to determine an output 630. In a training phase, the dataset 605 comprises training data. In an inference phase, the dataset 605 comprises the real-world data. For example, the real-world data may comprise patient-specific variants generated before or after a filtering step as shown in FIG. 3.

The dataset 605 may comprise sequencing data corresponding to variants. In some instances, the dataset 605 comprises paired sequencing data and variant data. The paired sequencing data and variant data may be obtained by sequencing nucleic acid at have different sequencing coverage or depth. For example, tissue samples may have a sequencing depth of (e.g., about 80×) due to the high abundance of nucleic acid that may can be isolated and/or sequenced from such the tissue samples. Noncancerous (e.g., normal) samples, such as tissue, cells, white blood cells, buffy coated cells buffy coat, etc., may be sequenced to a different depth (e.g., of about 40×), while other samples (e.g., non-tissue samples or, plasma samples) may achieve be sequenced at a sequencing depth (e.g., about 30×) that is different from the depths above of about 30× due to the limit abundance of nucleic acid material. Differences in sequencing depth may affect the overall quality of sequencing results and variant calling. A same set of sequencing depths may be used in the training phase and the inference phase with regard to obtaining the dataset 605. In some instances, different sets of sequencing depths are used in the training phase and the inference phase. Accordingly, In some instances, the paired sequencing and variant data of the dataset 605 accessed by data generator 405 may also be generated by diluting samples by different dilution factors levels to various DNA concentrations and sequencing the diluted samples. For example, the biological samples (e.g., tissue samples, noncancerous samples, and/or non-tissue samples) may have a DNA concentration of about 0 to about 1×10-10. More specifically, the data samples may have a DNA concentration of diluted at a dilution level of about 0.01, about 0.001, about 5×10⁻⁴, about 2×10⁻⁴, about 1×10⁻⁴, about 5×10⁻⁵, and or about 1×10⁻⁵.

Each decision tree 610 is a decision support tool that uses a binary tree graph to make decisions and/or predict their possible consequences. In training a random forest, each decision tree is constructed independently based on a random subset of the training data and a random subset of the features (“bootstrapping”). When constructing each decision tree 610, instead of considering all features of a data point (e.g., a variant) for each split, a random subset of features (Nᵢ features 615) is generally selected, which helps introduce randomness and diversity among the decision trees in the forest. In addition to randomly selection of Ni features 615, each decision tree is also trained on a bootstrap sample of the training data, which can be a random sample of the same size as the original dataset but with replacement. This means that some samples (e.g., variants) may be included multiple times, while others may be left out in training a specific decision tree. Each decision tree may be seen as embodying a number of yes/no questions to assess the probability whether a variant is a true positive variant that is indicative of a positive ctDNA status. Each tree generates its own variant score independent of the other trees in the ensemble model. Random forest may later use a voting scheme 610 (e.g., majority voting or soft voting) to ensemble the decision trees 610 and determine a final classification, a final score, or a ctDNA status for the sample associated with the dataset 605. The training of the random forest machine learning model 600 and/or the decision trees 610 can be performed using the training and validation subsystem 415 described with respect to FIG. 4. The number of decision trees n may be a hyperparameter that is provided before the training. It should be understood that each decision tree 610 does not need to be a balanced tree with equal number of nodes on the left branch and right branch, and it may have a different depth than the depth as shown in FIG. 4.

Each data point (e.g., a variant with corresponding features) in the dataset 605 traverses down each of the decision trees 610 that make up the random forest model. The random forest machine learning model 600 may comprise at least several hundred decision trees (e.g., n≥500 or n≥1,000) with each one contributes weakly to the classification, but as an ensemble, the random forest machine learning model 600 is a strong classifier. For example, the decision trees 610 may take about 10 features to about 500 features into consideration, with each decision tree takes a different subset of features (e.g., the Nᵢ features 615) into consideration. In some instances, the total number of features the decision trees considered for each variant may be 62. In some instances, the number is at least 62. It should be understood that more or less features may be considered.

In some instances, each decision tree 610 generates a score for each variant in the dataset 605, and the score is a value between [0, 1]. In some instances, the score is a binary score of either 0 or 1 (i.e., a classification score). In some instances, each decision tree 610 is configured to generate a score for all variants in the dataset 605, and the score is either a value between [0, 1] or a binary score of either 0 or 1.

If a feature used for a splitting is missing from the dataset 605, different techniques may be used to fix the missing. For example, surrogate splitting may be used when a feature is missing for a data point in the training subset of data, the decision tree is configured to use another feature that is correlated with the missing feature to make a decision. The surrogate feature is typically the feature that best mimics the split that the missing feature would have caused if it were available. If a suitable surrogate feature is not available, the decision tree may use the most common value of the missing feature in the training data, or it may use a default value. If a feature is missing during the inference phase, imputation may be used to replace the missing value with a substitute value. The substitute value may be configured during the training to be a mean, a median, or a mode of the feature in the training dataset. Surrogate splitting can be used to select another feature that is correlated with the missing feature to make the split. In some instances, the random forest machine learning model may be configured to have a default path to deal with the missing situation.

The scores generated by the decision trees 610 can be ensembled based on a voting scheme 620. In some instances, the voting scheme 620 includes a majority voting. In the majority voting scheme, each decision tree in the random forest generates a classification score of a given variant, and the final classification is the class (e.g., 0 or 1) that receives the most “votes” from each individual tree. In some instances, the voting scheme 620 includes a soft voting, which calculates an average score from all the decision trees and/or selects the class with the highest average probability as the final classification. The final classification may be provided as the output 630. In some instances, the random forest machine learning model is configured to generate a final score for each subject based on all variants in the dataset 605, and the score is normalized final classification across all variants in the dataset 605. The final score can be also provided as a part of the output 630. A part or all input data in the dataset 605 may also be provided as a part of the output 630.

Referring back to FIG. 5B, at box 545, the classification model (e.g., random forest classification model) is trained with some number of trees. The training is an iterative process that starts at the first node of the first tree and comprises initially inputting a portion of the labeled training data into the classification model. The portions of labeled training data are sampled at random with replacement to create a subset of training data (e.g., also known as bootstrapping resampling). The subset may be, for example, about 66% of the total training dataset. At each node: (i) a number of variant features from the portion of the labeled training dataset are selected; (ii) using an objective function, it is determined which of the variant features from the number of variant features provides the best binary split. The best split is based on which feature variant feature minimizes the objective function; (iii) the first node is assigned the determined variant feature; (iv) and the iterative process is repeated at the second and subsequent nodes of the first tree for a number of iteration or epochs until the first tree is generated. This process, steps (i)-(iv) are repeated for the first node of a second and subsequent tree until all the variant features have been assigned to a tree. In other words, the random forest trees are constructed from the parameters/features of the data it is trained on (e.g., variant features). The types of features that may be used include FASTQ quality score, alignment score, read coverage, strand bias, and the like. Further, some optimal number of variant features is preferably discovered. Random forest runtimes are fast, and they can deal with unbalanced and missing data.

For a random forest, generally the number of variant features << number of predictor variables. When running a random forest, when a new input (e.g., variant) is entered into the system, it traverses down all the trees. The result may either be an average or weighted average of all the terminal nodes that are reached. With many predictors, the eligible predictor set will be different from node to node. As the number of variant features goes down, both inter-tree correlation and the strength of individual trees go down.

At block 550, the trained classification model is output that generates variant scores for the variants in the labeled training dataset. The classification model can apply various filtering and scoring techniques to ensure only high confident variants are considered. Further, the filtering and scoring techniques may function as a pass-through criterion with minimum values or ideal ranges to ensure high quality candidate alterations are considered. In other words, the trained classification model, using various filters and thresholds, will robustly remove any false positive variants and low-quality variants.

After the variant scores are generated by the classification model, they can be used to determine the status (e.g., presence or absence) of ctDNA in the non-tissue sample as well as estimate a level of ctDNA in the non-tissue sample. To determine the status of ctDNA, all the variant scores for the non-tissue sample are summed up and divided by the total number of candidate somatic variants to give a normalized variant score. The normalized variant score may be used as the primary measure for detection of cancer (e.g., whether the non-tissue sample is ctDNA+ or ctDNA−). A non-tissue sample is considered ctDNA+ when the normalized variant score is greater than or equal to the maximum normalized variant score plus one standard deviation of the reference cohort variants.

A ctDNA level for the non-tissue sample is determined by taking the total number of distinct overlapping variant reads, where the variant has a scores greater than 0.25, over the sum of (1) distinct overlapping reads per observed variant and (2) the product of the median genome wide distinct overlapping read coverage with the total unobserved candidate somatic variants to give an estimated ctDNA fraction (as a percent). In other words, the estimated ctDNA level represents a proportion of the total cfDNA collected from the patient.

### VI. COMPUTING ENVIRONMENTS: MACHINES, SOFTWARE AND INTERFACES

Certain processes and methods described herein (e.g., mapping, counting, normalizing, range setting, adjusting, categorizing and/or determining sequence reads, counts, levels and/or profiles, ctDNA detection and analysis, and the like) are performed within a computing environment comprising a computer, microprocessor, software, module, other machines such as sequencers, or combinations thereof. The methods described herein typically are computer-implemented methods, and one or more portions or steps of the method are performed by one or more processors (e.g., microprocessors), computers, systems, apparatuses, or machines (e.g., microprocessor-controlled machine). Computers, systems, apparatuses, machines, and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

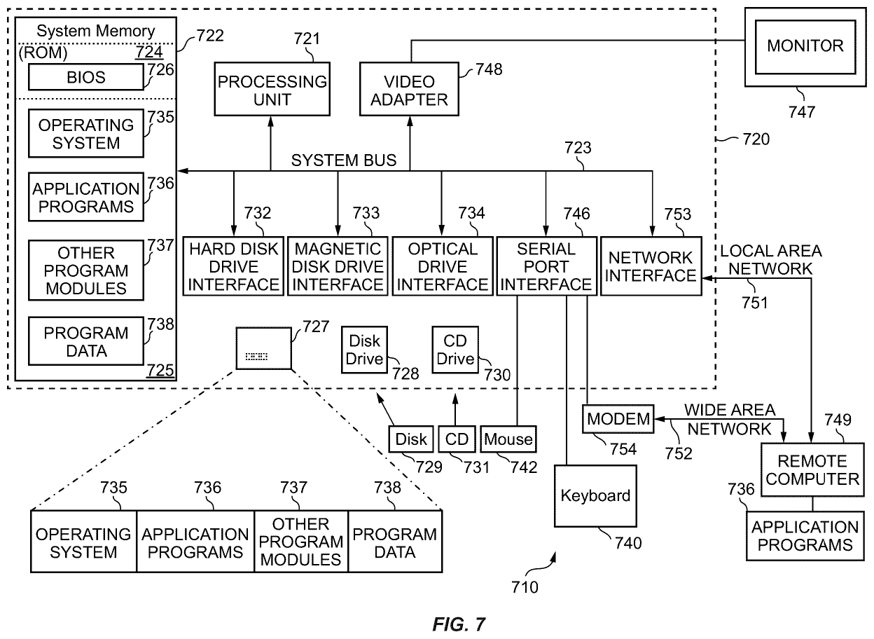

FIG. 7 illustrates a non-limiting example of a computing environment 710 in which various systems, methods, process, and data structures described herein may be implemented. The computing environment 710 is only one example of a suitable computing environment and is not intended to suggest any limitation as to the scope of use or functionality of the systems, methods, and data structures described herein. Neither should computing environment 710 be interpreted as having any dependency or requirement relating to any one or combination of components illustrated in computing environment 710. A subset of systems, methods, and data structures shown in FIG. 7 can be utilized in certain embodiments. Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment 710 includes a computing device 720 (e.g., a computer or other type of machines such as sequencers, photocells, photo multiplier tubes, optical readers, sensors, etc.), including a processing unit 721, a system memory 722, and a system bus 723 that operatively couples various system components including the system memory 722 to the processing unit 721. There may be only one or there may be more than one processing unit 721, such that the processor of computing device 720 includes a single central-processing unit (CPU), or a plurality of processing units, commonly referred to as a parallel processing environment. The computing device 720 may be a conventional computer, a distributed computer, or any other type of computer.

The system bus 723 may be any of several types of bus structures including a memory bus or memory controller, a peripheral bus, and a local bus using any of a variety of bus architectures. The system memory may also be referred to as simply the memory and includes read only memory (ROM) 724 and random access memory (RAM) 725. A basic input/output system (BIOS) 726, containing the basic routines that help to transfer information between elements within the computing device 720, such as during start-up, is stored in ROM 724. The computing device 720 may further include a hard disk drive 727 for reading from and writing to a hard disk, not shown, a magnetic disk drive 728 for reading from or writing to a removable magnetic disk 729, and an optical disk drive 730 for reading from or writing to a removable optical disk 731 such as a CD ROM or other optical media.

The hard disk drive 727, magnetic disk drive 728, and optical disk drive 730 are connected to the system bus 723 by a hard disk drive interface 732, a magnetic disk drive interface 733, and an optical disk drive interface 734, respectively. The drives and their associated computer-readable media provide non-volatile storage of computer-readable instructions, data structures, program modules and other data for the computing device 720. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

A number of program modules may be stored on the hard disk 727, magnetic disk 728, optical disk 730, ROM 724, or RAM 725, including an operating system 735, one or more application programs 736, other program modules 737, and program data 738. A user may enter commands and information into the computing device 720 through input devices such as a keyboard 740 and pointing device (e.g., mouse) 742. Other input devices (not shown) may include a microphone, joystick, game pad, satellite dish, scanner, or the like. These and other input devices are often connected to the processing unit 721 through a serial port interface 746 that is coupled to the system bus 723, but may be connected by other interfaces, such as a parallel port, game port, or a universal serial bus (USB). A monitor 747 or other type of display device is also connected to the system bus 723 via an interface, such as a video adapter 748. In addition to the monitor 747, computers typically include other peripheral output devices (not shown), such as speakers and printers.

The computing device 720 may operate in a networked environment using logical connections to one or more remote computers, such as remote computer 749. These logical connections may be achieved by a communication device coupled to or a part of the computing device 720, or in other manners. The remote computer 749 may be another computer, a server, a router, a network PC, a client, a peer device, or other common network node, and typically includes many or all of the elements described above relative to the computing device 720, although only memory storage devices has been illustrated in FIG. 7. The logical connections depicted in FIG. 7 include a local-area network (LAN) 751 and a wide-area network (WAN) 752. Such networking environments are commonplace in office networks, enterprise-wide computer networks, intranets, and the Internet, which all are types of networks.

When used in a LAN-networking environment, the computing device 720 is connected to the LAN 751 through a network interface or adapter 753, which is one type of communications device. When used in a WAN-networking environment, the computing device 720 often includes a modem 754, a type of communications device, or any other type of communications device for establishing communications over the WAN 752. The modem 754, which may be internal or external, is connected to the system bus 723 via the serial port interface 746. In a networked environment, program modules depicted relative to the computing device 720, or portions thereof, may be stored in the remote memory storage device. It is appreciated that the network connections shown are non-limiting examples and other communications devices for establishing a communications link between computers may be used.

### VII. EXAMPLES

Illustrative examples of the invention are provided in the working examples and further illustrate the advantages and features of the present invention but are not intended to limit the scope of the invention. While these examples are typical of those that might be used, other procedures, methodologies, or techniques known to those skilled in the art may alternatively be used.

### Purpose and Translational Relevance

Surgery followed by adjuvant chemotherapy (ACT) is standard of care practice for patients with stage III colon cancer. ACT decisions for non-metastatic colon cancer are currently based on clinicopathological risk factors. All patients with stage III colon cancer are eligible for ACT, even though more than 50% are cured by surgery alone. Further, of the patients who are administered ACT, only 15-20% benefit from ACT, while all patients are exposed to the risk of developing considerable side effects. Therefore, there is an urgent unmet need to identify those stage III colon cancer patients that are truly at risk of recurrence after surgery and could benefit from ACT. Liquid biopsy circulating tumor DNA (ctDNA) detection after resection of the primary tumor allows patients with micro-metastatic disease who are at high-risk of experiencing disease recurrence to be identified.

CtDNA-based minimal residual disease (MRD) detection is a strong prognostic biomarker for disease recurrence in stage II and III colon cancer. MRD detection post-surgery is technically demanding due to extremely low levels of ctDNA. Tumor-informed WGS approaches hold promise for MRD testing, given the ability to track thousands of tumor-specific mutations without the need for personalized assay development. However, the clinical performance of these methods remains to be fully established. Here, a novel, tumor-informed WGS-based approach for detecting MRD is described herein. The Prospective Dutch ColoRectal Cancer cohort (PLCRC) sub-study PROVENC3 aimed to determine the clinical validity of post surgery ctDNA status to predict recurrence within three years in patients with stage III colon cancer treated with ACT.

The PROVENC3 study determined the clinical validity of a novel whole genome sequencing-based ctDNA detection assay in adjuvant chemotherapy-treated stage III colon cancer patients. Combining ctDNA test results with established clinicopathological risk factors allowed patients to be distinguished into groups that are at either a very low risk or a very high-risk of developing a recurrence within 3 years. These data have broad implications for altering current clinical practice treatment plans and enable the design of ctDNA-guided interventional (de-)escalation trials that aim to improve disease management of patients with stage III colon cancer.

### Brief Overview of Experimental Design

Blood was collected pre-surgery, post-surgery and post-ACT. Tumor-informed plasma ctDNA detection was performed through integrated whole genome sequencing (WGS) analyses of formalin-fixed paraffin-embedded tumor tissue DNA (80×), white blood cell germline DNA (40×) and plasma cell-free DNA (30×).

### Materials and Methods

### 1. PROVENC3 Study Population: PLCRC Infrastructure

Patients, diagnosed with colorectal cancer, aged 18 years or older and mentally competent, were recruited in both academic and non-academic hospitals in the Netherlands for participation in the ongoing Prospective Dutch ColoRectal Cancer cohort (PLCRC, NCT02070146). Informed consent for the collection of long-term clinical and survival data was mandatory for participation in PLCRC. Subsequently, patients were given the option to consent to: 1) filling out questionnaires on 205 health related quality of life, functional outcomes, and workability; 2) biobanking of tumor and normal tissue; 3) collection of blood samples; and 4) to be offered studies conducted within the infrastructure of the cohort. Treatment naïve non-metastatic colorectal cancer (CRC) patients that gave informed consent for PLCRC and for additional blood sampling were included in the observational PLCRC sub-study, MEDOCC (Molecular Early Detection of Colon Cancer). Patients with stage III colon cancer who started adjuvant chemotherapy (ACT) after surgery and for whom post-surgery blood was available were included in the PLCRC-MEDOCC sub-study PROVENC3 in 26 hospitals from 2016 to 2021. One stage III rectal cancer patient treated as colon cancer (cap(ox)/no radiation) was also included in the cohort. Clinical data was collected via the Netherlands Cancer Registry and through site visits by the PLCRC study team.

The PLCRC study was performed in accordance with the Declaration of Helsinki and approved by a medical ethical committee (Central Committee on Research Involving Human Subjects, CCMO: NL47888.041.14). All patients signed written informed consent for study participation and collection of blood and tissue samples for translational research. The PLCRC sub-study PROVENC3 was approved by the institutional review board (IRB) of the Netherlands Cancer Institute, Amsterdam, the Netherlands (protocol CFMPB472).

### 2. Sample Collection and Processing

Formalin-fixed, paraffin embedded (FFPE) tumor blocks were requested through PALGA, the nationwide network and registry of histopathology and cytopathology in the Netherlands. The hematoxylin and eosin (H&E) slides were evaluated by a pathologist, and the “tumor area” was outlined on the slide for macro-dissection. DNA was isolated from FFPE slides using the QIAGEN AllPrep DNA/RNA FFPE kit (QIAGEN, Hilden, Germany) and stored at −20° C. or 4° C. only for short term before shipment. DNA quality and quantity were measured on a Nanodrop One (Isogen, Ijsselstein, The Netherlands) and on a Qubit 3.0 Fluorometer (Molecular Probes, Leiden, The Netherlands) with the use of the Qubit dsDNA High-Sensitivity Assay (Thermo Fisher Scientific, USA).

Blood samples were collected pre-surgery, post-surgery before the start of adjuvant chemotherapy, after completion of adjuvant chemotherapy and every 6 months for up to 3 years. Blood was collected using a cell stabilizing BCT tube (Streck, La Vista, NE) in the participating hospitals and shipped to the Netherlands Cancer Institute. Cell-free plasma and white blood cells (WBC) were separated by centrifugation of the blood for 10 minutes at 1,700×g followed by 10 minutes at 20,000×g, then stored at −80° C. until further processing. Cell-free DNA (cfDNA) was isolated from the available plasma using the QIAsymphony DSP Circulating DNA Kit (QIAGEN, Hilden, Germany) with a fixed elution volume of 60 μL. Genomic DNA was isolated from WBCs using the QIAsymphony DSP DNA Midi Kit (QIAGEN, Hilden, Germany) and 1 mL blood protocol. cfDNA and genomic DNA from WBCs was stored at −20° C. until further processing. The Qubit dsDNA High-Sensitivity Assay (Thermo Fisher, Waltham, MA) was used to quantify DNA yield for next generation sequencing. Samples were de-identified and blinded, then shipped to Personal Genome Diagnostics (Labcorp, Baltimore, MD) for sample testing and analysis. Post surgery ctDNA was evaluated for all patients in the cohort. Pre-surgery ctDNA was evaluated for 18 out of 22 of the post-surgery ctDNA-positive patients with blood available and a random selection of 33 patients from the remaining cohort. Post-ACT ctDNA was evaluated for 13 out of 22 of the post-surgery ctDNA-positive patients with blood available.

### 3. Noncancerous Donor Plasma and Commercial Cell Line Cohorts

All patients provided written informed consent and the studies were performed according to the Declaration of Helsinki. Noncancerous donor plasma samples were obtained under Institutional Review Board approval from Discovery Life Sciences (Alabama, USA). Human tumor and normal cells from previously characterized cell lines were obtained from ATCC (Virginia, USA) (COLO-829, HCC-1187, HCC-1143, HCC-1954) and SeraCare (Massachusetts, USA) (SeraSeq gDNA TMB-mix Score 26). cfDNA was isolated from plasma using the Qiagen Circulating Nucleic Acid kit (Qiagen, Germany) and the concentration was assessed using the Qubit dsDNA High-Sensitivity Assay (Thermo Fisher, USA). Genomic DNA was isolated from cell line samples using the QIAamp DNA Blood Mini Kit (Qiagen, Germany) and the concentration assessed using the Qubit dsDNA Broad Range Assay (Thermo Fisher, USA).

### 4. NGS Analysis of Tumor Tissue Derived and Germline Genomic DNA

Genomic DNA was quantified using the Qubit dsDNA Broad Range Assay (Thermo Fisher, USA) and up to 400 ng of DNA was sheared to a target fragment size of approximately 450 base pairs (bp) using Covaris focused ultrasonication (Covaris, USA). Additionally, genomic DNA derived from FFPE tumor tissue was repaired using the PreCR Repair Mix (New England Biolabs, USA). Whole-genome next-generation sequencing libraries were prepared from fragmented genomic DNA through end-repair, A-tailing, and adapter ligation with the KAPA HyperPrep reagent kit according to the manufacturer's protocol (Roche, USA). Subsequently, these libraries were amplified through 7 cycles of polymerase chain reaction (PCR), pooled, and sequenced with 150 bp paired-end reads using the Illumina NovaSeq6000 platform (Illumina, USA) to a target depth of 80× for tumor samples and 40× for germline samples. After demultiplexing was performed using bcl2fastq (Illumina, USA), FASTQ files were aligned to the GRCh38 human reference genome using BWA-MEM (v0.7.15). PCR duplicates were marked using Novosort (v1.03.01) and base quality score recalibration was performed using GATK BQSR (v4.1.0). The aligned BAM files were subjected to single nucleotide variant (SNV) analyses using MuTect2 (GATK v4.0.5.1), Strelka2 (v2.9.3), and Lancet (v1.0.7). SNVs were annotated as high confidence if they were reported by at least two variant callers.

5. NGS Analysis of Plasma Derived cfDNA and Contrived DNA

cfDNA and contrived DNA obtained from fragmented matched tumor and germline cell lines were quantified using the Qubit dsDNA High-Sensitivity Assay (Thermo Fisher, USA). Whole genome next generation sequencing libraries were prepared from cell-free or contrived DNA using a target of 10 ng of DNA through end-repair, A-tailing, and adapter ligation with custom molecular barcoded adapters. Subsequently, these libraries were amplified through 5 cycles of PCR, pooled, and sequenced with 150 bp paired-end reads using the Illumina NovaSeq6000 platform (Illumina, USA) to a target depth of 30×. After demultiplexing was performed, FASTQ files were quality trimmed using Trimmomatic (v0.33) and aligned to the hg19 human reference genome using BWA-MME2 (v2.2.1). Somatic variant identification was performed using VariantDx (v11.0.0), which has demonstrated high accuracy for somatic mutation detection and differentiating technical artifacts to enable analyses of SNVs.

### 6. Detection of Tumor DNA Through WGS Analysis

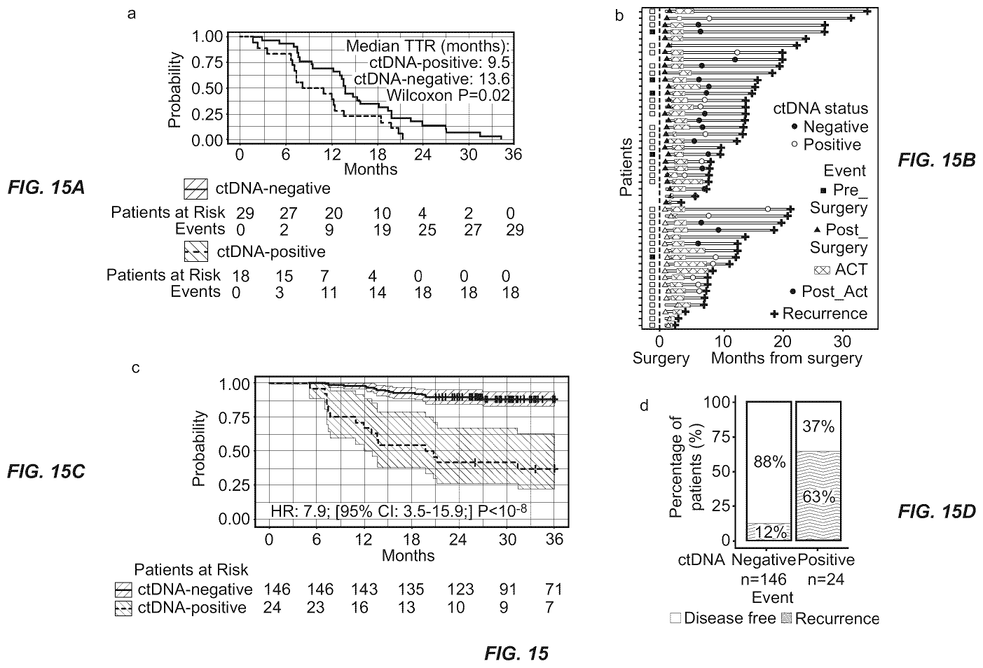

Initially, to ensure that the tumor, germline, and plasma WGS datasets were derived from the same subject, an analysis was performed across 10,000 common single nucleotide polymorphisms. Then, a quality control analysis was performed using Picard (v2.18.14) and required ≥20× sequencing depth with a median insert size ≥150 bp for cfDNA samples, ≥40× sequencing depth for tumor samples, and ≥20× sequencing depth for germline samples. Tumor-specific single nucleotide variants were filtered to a candidate somatic mutation set by removing: (1) variants observed in the 1000 Genomes (Phase 3) or gnomAD (r2.0.1) population databases, (2) variants overlapping 296 the hg19 UCSC simple tandem repeat tracks, (3) positions with <10× depth in the tumor or matched normal, (4) positions with an alternate allele count <4 in the tumor or >1 in the matched germline, and (5) variants with a tumor variant allele frequencies (VAF) <0.05 (more strict filtering was applied to T>C/A>G variants, which were removed if the tumor VAF was <0.20 or the alternate allele count was <10). Additional variant filtering was performed through generation of a blacklist, where variants were further removed if present (1) in >10% of noncancerous donors or (2) any noncancerous donor contained the variant with ≥25% VAF across a cohort of 20 noncancerous donor plasma samples evaluated in quadruplicate (n=80 total). The final candidate tumor-specific variant set was then compared to the matched test sample unfiltered variant results. Candidate tumor specific SNVs identified in the test sample were scored (ranging from 0 to 1) using a random forest machine learning algorithm trained using the caret package (v6.0.90) within the R statistical computing environment (v4.1.1), independently of the PROVENC3 cohort. To avoid overfitting, model training utilized 5-fold cross validation and limited the number of selected variables per split procedure (hyperparameter mtry) to the square-root of the total number of input features. Variants present in properly paired mapped fragments with a random forest score >0.25 were further assessed, requiring an alternate read mapping quality ≥30 and a read-based mutation rate ≤5. The individual variant random forest scores were then aggregated and normalized based on the total number of tumor specific SNVs assessed. The normalized random forest score (NRFS) was then compared to the noncancerous donor cohort, and a cutoff of one standard deviation above the maximum observed NRFS was required to report an individual test sample as having evidence of the tumor-specific variants. An estimated tumor fraction (termed “Aggregate ctDNA VAF”) was then calculated for each positive test sample based on the aggregate variant allele observations observed as a proportion of the total unique coverage of all individual tumor-specific variants assessed. Analytical sensitivity of the tumor-informed WGS approach was assessed using five commercially available cell lines evaluated across a four-log tumor content range and demonstrated a limit of detection (95%) of 0.005% 320 tumor content and limit of detection (50%) of 0.001% tumor content. Furthermore, the observed tumor fraction was also highly correlated with the reference tumor fraction (Pearson correlation coefficient=0.96, p<0.001). Analytical specificity was determined through analysis of 119 noncancerous donor plasma specimens evaluated against 17 reference whole-genome somatic mutation datasets and demonstrated a specificity of 99.6% (2,015/2,023). Finally, analysis of an external contrived reference control sample demonstrated highly reproducible results for the estimated tumor fraction across 24 independent runs evaluated for the PROVENC3 clinical study (n=45, CV=7.2%) See FIG. 15A-15D.

### 7. Statistical Analysis

Differences in baseline characteristics for the groups compared were analyzed using Fisher's exact test for categorical variables, and Mann-Whitney test for continuous variables. Post surgery ctDNA-positive vs post-surgery ctDNA-negative in the complete cohort, the primary outcome measure was “time to recurrence” as defined in Cohen et al. The only events considered were the recurrences. For time-to-event analyses, patients were censored at the last time point with follow up information available without a recurrence being reported, or at 36 months if FU was longer. Patients without an event, but with available follow-up of less than one year were excluded from the analysis. For univariate time-to-event analyses, we used the Kaplan Meier estimator and fitted Cox regression models. The clinicopathological variables evaluated were selected based on clinical relevance: Clinicopathological risk status (Low risk=T1-3N1, High risk=T4 and/or N2), T status determined by the pathology report (T1-3, T4), N status determined by the pathology report (N1, N2), and microsatellite instability (MSI) status determined by next-generation sequencing of the primary tumor (stable, unstable). The change in the hazard ratios was also evaluated after stratifying each of the clinicopathological covariates based on post-surgery ctDNA status (Clinicopathological risk+ctDNA status, T status+ctDNA status, N status+ctDNA status, MSI status+ctDNA status). Kaplan Meier estimator curves were also fitted for these models.

Furthermore, we evaluated whether post-surgery ctDNA status had added and independent predictive value for recurrence in addition to the clinicopathological variables. We fitted several (multivariate) Cox regression models. First, the added value of ctDNA status was determined by fitting multivariate models combining the clinicopathological risk factors and ctDNA status and performing likelihood ratio test among them (LRT 1: Clinicopathological risk vs Clinicopathological risk+ctDNA status, LRT 2: Clinicopathological risk+MSI status vs Clinicopathological risk+MSI status+ctDNA status, LRT 3: T status+N status vs T status+N status+ctDNA status, LRT 4: T status+N status+MSI status vs T status+N status+MSI status+ctDNA status). Second, we evaluated the independent predictive value in the model of each variable by exploring the Hazard Ratios of each variable independently in the two best models resulting from the likelihood ratio tests:(Model 1: Clinicopathological risk+MSI status+ctDNA status, Model 2: T status+N status+MSI status+ctDNA status). All statistical and survival analysis were performed using R package “survival” for survival (R version 4.2.1).

### 8. Machine Learning Model Training

The random forest model was trained using the caret package (v6.0.90) within the R statistical computing environment (v4.1.1). A set of 62 features was provided for model training for 1,000 true positive mutations and 1,000 false positive mutations from the COLO-829 cell line across dilution levels of 0.01, 0.001, 5×10−4, 2×10−4, 1×10−4, 5×10−5, and 1×10−5. To avoid overfitting, model training utilized 5-fold cross validation and limited the number of selected variables per split procedure (hyperparameter mtry) to the square-root of the total number of input features.

The feature set includes: (‘A T count’, ‘AverageQualityScore’, ‘BaseFrom.ToAtoG’, ‘BaseFrom.ToAtoT’, ‘BaseFrom.ToCtoA’, ‘BaseFrom.ToCtoG’, ‘BaseFrom.ToCtoT’, ‘BaseFrom.ToGtoA’, ‘BaseFrom.ToGtoC’, ‘BaseFrom.ToGtoT’, ‘BaseFrom.ToTtoA’, ‘BaseFrom.ToTtoC’, ‘BaseFrom.ToTtoG’, ‘DistinctCoverage’, ‘DistinctNoOlapMuts’, ‘DistinctOlap1Mut’, ‘DistinctOlapMuts’, ‘DistinctOlapReads’, ‘DistinctPairs’, ‘DistMutPairsEORA’, ‘DistMutPairsEORAplusB’, ‘DistMutPairsEORB’, ‘Dust’, ‘DustRaw’, ‘EORMutPct’, ‘F1R2Mut’, ‘F2R1Mut’, ‘Forward’, ‘GCcount’, ‘MaskedMutPct’, ‘MaskedPairs’, ‘MMAvg’, ‘MMTumor’, ‘MutCountCov’, ‘MutMMAvg’, ‘MutPct’, ‘NonMutFwd’, ‘NonMutRev’, ‘NoOlapMut’, ‘NumAlleles’, ‘Olap1Mut’, ‘OlapMuts’, ‘OlapReads’, ‘PolyMut’, ‘PolyN’, ‘PolyNN’, ‘PolyNNN’, ‘ProperPairs’, ‘ProperPairsPct’, ‘Reverse’, ‘RMDScore’, ‘RptMask’, ‘SBFisherLeft’, ‘SBFisherRight’, ‘SBFisherTwotail’, ‘SBMutProportion’, ‘SBNonMutProportion’, ‘SBPropDelta’, ‘TumMutRMSMAPQ’, ‘TumNERDistMean’, ‘TumNERDistSD’, ‘TumRMSMAPQ’).

### Example 2: PROVENC3 Study Overview

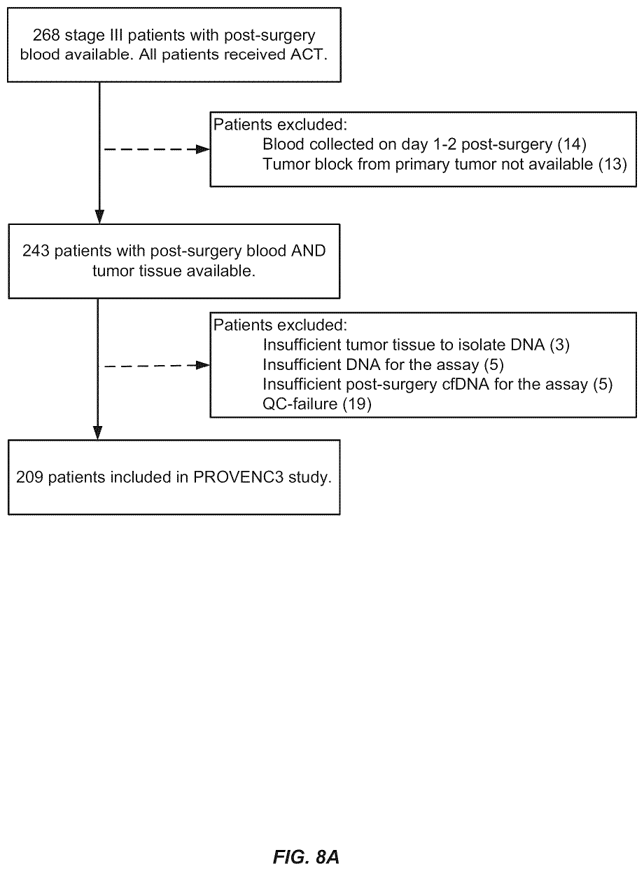

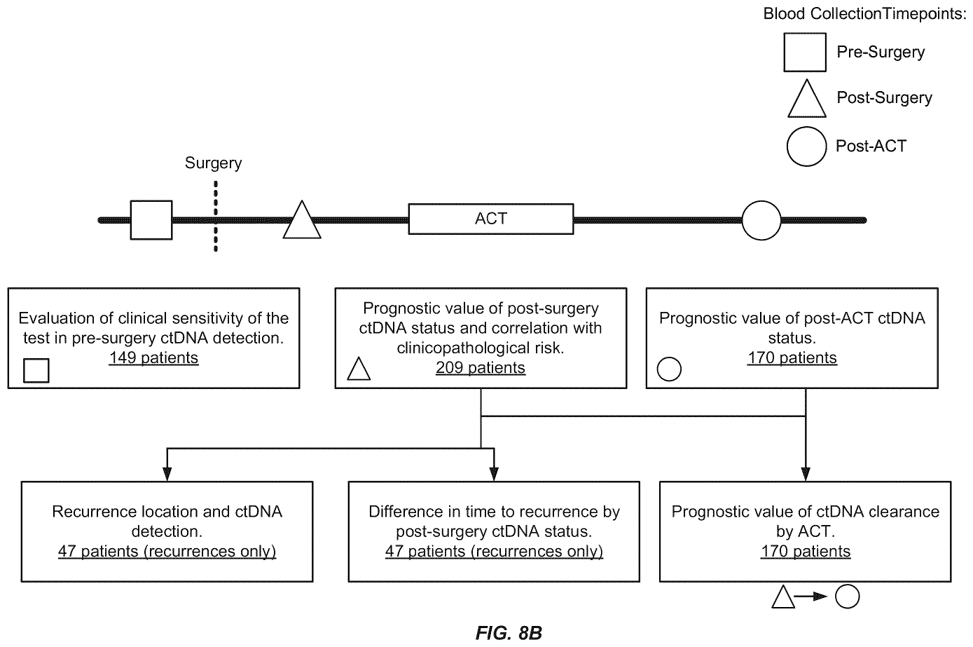

FIG. 8A provides an overview of the PROVENC3 study population along with the main exclusion criteria used to obtain the final patient population used for final analysis. Initially, 268 stage III colon cancer patients who received ACT and had post-surgery blood available were assessed for inclusion. Patients whose blood was collected on day 1 or 2 post-surgery (14) and/or whose primary tumor sample was not available (13) were removed. After the first filtering step, 243 patients remained where post-surgery blood and tumor tissue were available. Of those 243 patients, those whose tumor tissue isolated DNA was insufficient (3), total isolated DNA for the assay was insufficient (5), whose post-surgery cfDNA sample was insufficient (5), or whose variant profiles did not pass quality control (19) were also excluded from the study. As a result, 209 stage III ACT-treated colon cancer patients with a median follow-up of 40 months (interquartile range (IQR) 29-48 months) were included.

FIG. 8B shows an exemplary schematic approach to the PROVENC3 (PROgnostic Value of Early Notification by ctDNA in Colon Cancer stage 3) study. After informed consent, blood was collected pre-surgery and post-surgery. During surgery, a tumor sample was collected for FFPE preparation, and a blood sample was collected post-surgery from 3-65 days. The average (median) day of collection was 13 days, and the interquartile range (IQR) for collection was 4-20 days post-surgery. After surgery, all patients received ACT and post-ACT blood samples were collected every six months for up to 3 years. All tissue and blood samples were sent to a central laboratory (The Netherlands Cancer Institute) and clinical data was collected in the Netherlands Cancer registry by IKNL.

Blood samples collected from 149 patients pre-surgery were used to evaluate the clinical sensitivity of the ctDNA detection test. It was found that 134 out of 149 patients were ctDNA-positive (90%), underscoring the high ctDNA test sensitivity.

Blood samples collected from 209 patients post-surgery were used to determine a prognostic value of post-surgery ctDNA status and were correlated with clinicopathological risk factors to predict the risk of recurrence. In total, 28 out of 209 (13%) patients were ctDNA-positive after surgery. The post-surgery median aggregate ctDNA variant allele frequency (VAF) was 0.035% (range 0.01%-3.13%). As shown in Table 2, none of the evaluated baseline clinicopathological features were significantly associated with post-surgery ctDNA status.

TABLE 2
Summary of baseline clinicopathological
characteristics for the PROVENC3 cohort.

| | ctDNA positive | | ctDNA negative | | Total | | p-value |
|---|---|---|---|---|---|---|---|
| | n | % | n | % | n | % | 0.05 sig |
| | 28 | | 181 | | 209 | 100 | |
| median age (years) | 68 (43-83) | | 63 (32-79) | | | | 0.11 |
| Sex | | | | | | | |
| Female | 9 | 32 | 88 | 49 | 97 | 46 | 0.15 |
| Male | 19 | 68 | 93 | 51 | 112 | 54 | |
| Clinicopathological risk | | | | | | | |
| Low risk | 17 | 61 | 110 | 61 | 127 | 61 | 1 |
| High risk | 11 | 39 | 71 | 39 | 82 | 39 | |
| T status | | | | | | | |
| T1 | 0 | 0 | 4 | 2 | 4 | 2 | |
| T2 | 3 | 11 | 26 | 14 | 29 | 14 | |
| T3 | 18 | 64 | 107 | 59 | 125 | 60 | |
| T4 | 7 | 25 | 44 | 24 | 51 | 24 | |
| T1-3 | 21 | 75 | 137 | 76 | 158 | 76 | 1 |
| T4 | 7 | 25 | 44 | 24 | 51 | 24 | |
| N status | | | | | | | |
| N1a | 6 | 21 | 48 | 27 | 54 | 26 | |
| N1b | 11 | 39 | 69 | 38 | 80 | 38 | |
| N1c | 2 | 7 | 9 | 5 | 11 | 5 | |
| N1m | 0 | 0 | 9 | 5 | 9 | 4 | |
| N2a | 5 | 18 | 26 | 14 | 31 | 15 | |
| N2b | 4 | 14 | 20 | 11 | 24 | 11 | |
| N1 | 19 | 68 | 135 | 75 | 154 | 74 | 0.49 |
| N2 | 9 | 32 | 46 | 25 | 55 | 26 | |
| MSS status | | | | | | | |
| Stable (MSS) | 26 | 93 | 153 | 85 | 179 | 86 | 0.38 |
| Instable (MSI) | 2 | 7 | 28 | 15 | 30 | 14 | |
| Resection | | | | | | | |
| Radical | 24 | 86 | 174 | 96 | 198 | 95 | 0.18 |
| Non radical | 2 | 7 | 4 | 2 | 6 | 3 | |
| UNK | 2 | 7 | 4 | 2 | 6 | 3 | |
| Histology | | | | | | | |
| Adenocarcinoma, NOS | 26 | 93 | 159 | 88 | 185 | 89 | 0.63 |
| Medullary carcinoma, NOS | 0 | 0 | 3 | 2 | 3 | 1 | |
| Mucinous adenocarcinoma | 1 | 4 | 17 | 9 | 18 | 9 | |
| Signet ring cell carcinoma | 1 | 4 | 2 | 1 | 3 | 1 | |
| Differentiation grade | | | | | | | |
| Well differentiated | 0 | 0 | 0 | 0 | 0 | 0 | 1 |
| Moderately differentiated | 22 | 79 | 149 | 82 | 171 | 82 | |
| Poorly differentiated | 4 | 14 | 25 | 14 | 29 | 14 | |
| Undifferentiated | 0 | 0 | 0 | 0 | 0 | 0 | |
| UNK | 2 | 7 | 7 | 4 | 9 | 4 | |
| Tumor location | | | | | | | |
| Left | 13 | 46 | 106 | 59 | 119 | 57 | 0.31 |
| Right | 15 | 54 | 75 | 41 | 90 | 43 | |
| Angioinvasion | | | | | | | |
| Extramural venous invasion | 6 | 21 | 34 | 19 | 40 | 19 | 0.66 |
| Intramural venous invasion | 1 | 4 | 5 | 3 | 6 | 3 | |
| No | 13 | 46 | 95 | 52 | 108 | 52 | |
| UNK | 8 | 29 | 47 | 26 | 55 | 26 | |
| RAS | | 0 | | | | | |
| WT | 13 | 46 | 113 | 62 | 126 | 60 | 0.15 |
| mut | 15 | 54 | 68 | 38 | 83 | 40 | |
| BRAF | | | | | | | |
| WT | 21 | 75 | 146 | 81 | 167 | 80 | 0.46 |
| mut | 7 | 25 | 35 | 19 | 42 | 20 | |

Abbreviations: MSS status (microsatellite stability status); MSS (microsatellite stable); MSI (microsatellite instable); UNK (unknown); WT (wild type); mut (mutant).

The clinicopathological risk factors were based on the patient's tumor pathological stage (T status) and their lymph node pathological state (N status). T status was assessed at stages 1-4. A T1 status indicates the tumor is only in the inner layer of the bowel. A T2 means the tumor has grown into the muscle layer of the bowel wall. A T3 means the tumor has grown into the outer lining of the bowel wall but has not grown through it. T4 means that the tumor has grown into the outler lining of the bowel wall and has spread to other tissue and/or organs. Patients with a clinicopathological risk factor of pT4pT4 are considered at high risk for recurrence while patients with a clinicopathological risk factor of pT1pT1 are considered at low risk of recurrence. N status was assessed at stage 1 and 2. As shown, N1 status is split into 3 stages—N1a, N1b and N1c. N1a means there are cancer cells in 1 nearby lymph node, N1b means there are cancer cells in 2 or 3 nearby lymph nodes, and N1c means the nearby lymph nodes don't contain cancer, but there are cancer cells in the tissue near the tumor. N1m means ______. N2 is split into 2 stages—N2a and N2b. N2a means there are cancer cells in 4 to 6 nearby lymph nodes and N2b means there are cancer cells in more than 7 nearby lymph nodes. Patients with a clinicopathological risk of pN2pN2 are considered high risk for recurrence while patients with a clinicopathological risk factor of 3N1 are considered low risk for recurrence.

As also shown in FIG. 8B, of the 209 patients 47 patients blood samples were used to assess the location of recurrence and to determine if there was a difference in time to recurrence based on post-surgery ctDNA status. Further, blood samples from 170 patients were evaluated for prognostic ctDNA status post-ACT as well as ctDNA clearance by ACT.

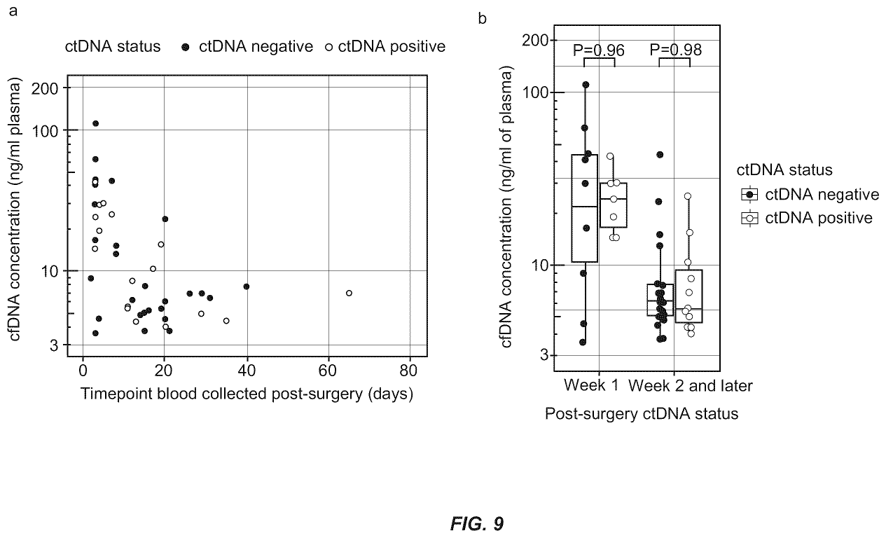

FIG. 9 shows a dot graph (FIG. 9A) and a box and whiskers (FIG. 9B) for post-surgery ctDNA status and cfDNA concentration. ctDNA-positive patients experiencing a recurrence were not underrepresented among blood samples with higher cfDNA levels that were drawn during week 1 (P=0.5). FIG. 9A shows an overview cfDNA concentration (ng/mL of plasma) plotted against the timepoint of post-surgery blood collection for 47 patients that experienced a recurrence. FIG. 9B illustrates that the cfDNA concentration did not differ between ctDNA-positive and ctDNA-negative patients, neither when blood was collected during the first week post-surgery (P=0.96) nor when collected during week 2 or later (P=0.98). The percentage of recurrences was similar for patients with blood collected during week 1 (26%) vs during week 2 and later (26%). The y axis shows the cfDNA concentration on a logarithmic scale. Abbreviations: cfDNA, cell free DNA; ng, nanograms; mL, milliliter.

### Example 3: Analytical Studies Validating ctDNA Detection Assay

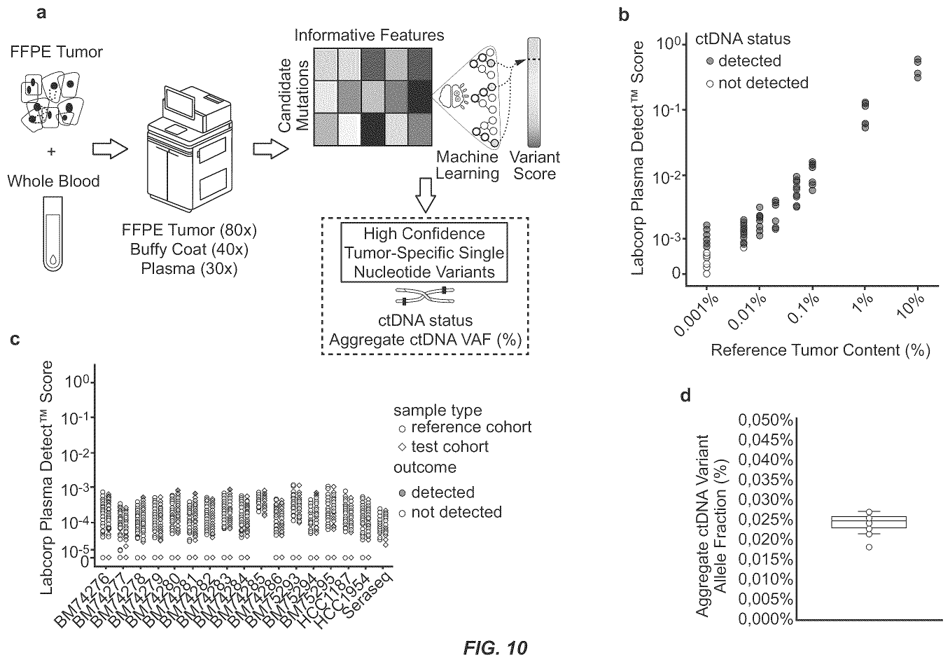

FIG. 10A shows an exemplary schematic of tumor-informed detection of ctDNA. To detect ctDNA, an integrated WGS analyses of patient-matched formalin-fixed paraffin embedded (FFPE) tumor tissue DNA, white blood cell-derived DNA and plasma cell-free DNA (cfDNA) using Labcorp Plasma Detect™ were utilized. Samples were sequenced to a depth of tumor tissue DNA (80×), germline DNA (40×), and plasma cell-free DNA (30×). WGS identified a median of 5,108 high confidence tumor specific single nucleotide variants (IQR 3,776-7,411) per patient, which were utilized for plasma ctDNA detection. Machine learning techniques (e.g., random forest classification) were used to generate variant scores. Further, informative features from candidate somatic variants were used to determine whether the plasma sample was ctDNA+ or ctDNA− and to estimate the level of ctDNA in the plasma sample.

FIG. 10B shows the results of analytical studies verifying the assay workflow described in FIG. 10A. Contrived reference models derived from five commercially available cell lines, including lung cancer (n=1), breast cancer (n=3), and melanoma (n=1) were used. Contrived samples were generated from three cell lines (COLO-829, HCC-1187, and HCC-1143) and evaluated in triplicate at 10%, 1%, 0.10%, 0.05%, 0.02%, 0.01%, 0.005%, and 0.001% tumor content. An additional contrived samples series (HCC-1187, HCC-1954) was generated and evaluated in triplicate at 0.05%, 0.01%, 0.005%, and 0.001% tumor content, along with the external contrived reference control sample (SeraSeq gDNA TMB-mix Score 26) evaluated at 0.05% (n=7), 0.01% (n=2), 0.005% (n=3), and 0.001% (n=3) tumor content to increase the number of datapoints near the expected limit of detection. These analyses demonstrated a limit of detection (95%) of 0.005% tumor content and a limit of detection (50%) of 0.001% tumor content.

FIG. 10C shows the results of the analytical specificity studies for the assay shown in FIG. 10A. The assay was found to have a specificity of 99.6% (2,015/2,023) across 119 noncancerous donor plasma specimens evaluated against 17 reference whole genome somatic mutation datasets.

FIG. 10D shows the results of the analytical reproducibility studies for the assay shown in FIG. 10A. Using the external contrived reference control samples, high reproducibility was observed across 24 independent runs evaluated for the PROVENC3 clinical study (CV=7.2%).

Table 3 below lists analytical study designs for limit of blank (LoB), limit of detection (LoD), and Clinical Confirmation studies. The LoB study was performed using a set of noncancerous donor plasma to determine the specificity of ctDNA detection. The LoD study was performed using cell line titrations to determine the lowest level that ctDNA can be confidently identified. The Clinical Confirmation study was performed using pre-surgical plasma to test the accuracy of ctDNA positivity calling in a set of clinical samples.

TABLE 3
Study Designs

| Study | N | Study Design |
|---|---|---|
| LoB | 120 | n = 40 noncancerous donor plasma samples in |
| | | triplicate |
| LoD (4 samples × | 60 | Cell line titrations in triplicate at 5 levels |
| 5 Levels) | | (0.05%, 0.01%, 0.005%, 0.001%, 0%) |
| Clinical | 14 | Commercial cohort (pre-surgery;- stage II |
| Confirmation | | and IV) 14 pre-surgical Plasma, tumor tissue |
| | | FFPE, and buffy coat (e.g., WBCs) sample sets |

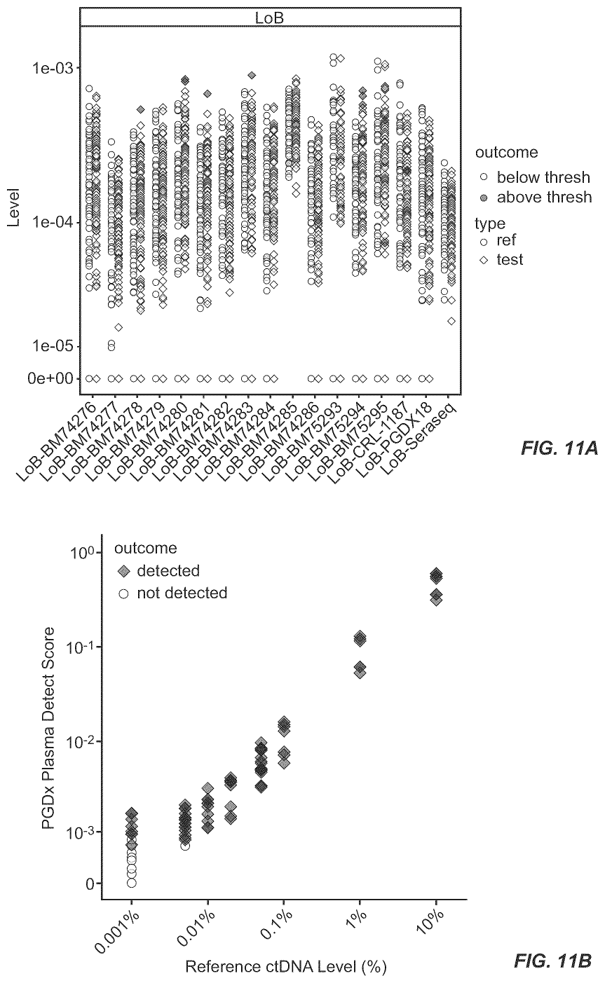

FIG. 11A shows the specificity evaluation of noncancerous donor plasma samples (n=119) against 17 reference somatic mutation datasets in one embodiment of the claimed invention. The reference somatic mutation datasets included 14 clinical FFPE tumor and 3 tumor cell line samples, including head and neck, colorectal, breast, and lung cancers. The reference tumor and normal samples as well as the noncancerous donor plasma cases were prepared, sequenced, and analyzed as detailed in the working example.

FIG. 11B shows results of analytical sensitivity studies for an embodiment of the claimed invention. The assay as claimed demonstrated a high sensitivity for ctDNA detection, with a limit of detection (95%) of 0.005% tumor content utilizing contrived reference models derived from commercially available cell lines including lung cancer, breast cancer, and melanoma. The observed tumor fraction also highly correlated with the reference tumor fraction (Pearson correlation coefficient=0.96, p<0.001). Cell line material was titrated with match normal to levels of 0.05%, 0.01%, 0.005%, 0.001%, and 0%, sheared to 160-170 bp, and subject to a 2-sided bead cleanup to simulate cfDNA. Contrived cell lines were prepared, sequenced, and analyzed as detailed in the working example.

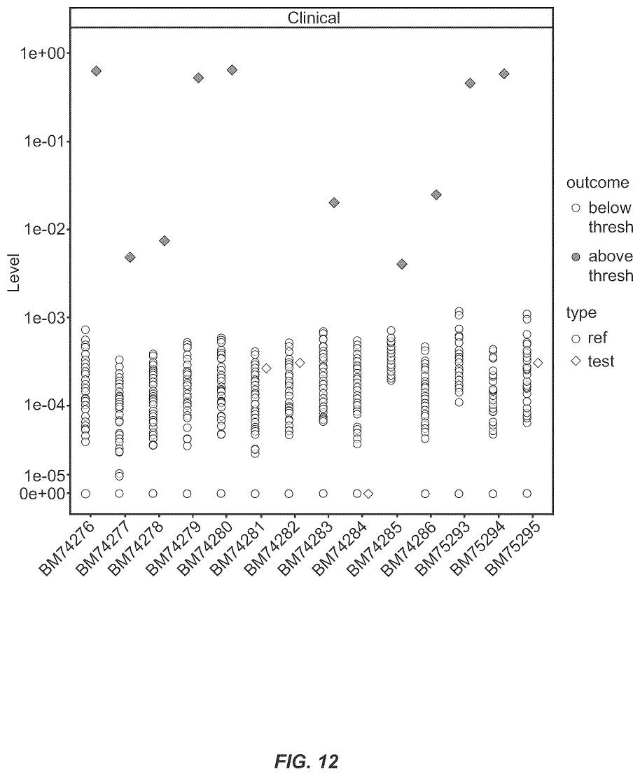

FIG. 12 shows the results of ctDNA detection across multiple solid tumors in an embodiment of the claimed invention. ctDNA was detected in 71% (10/14) of clinical samples (diamonds), significantly above background patient-specific reference levels established across an independent noncancerous donor cohort (circles). These clinical samples were obtained prior to surgical intervention across patients with stage II and IV colorectal and head and neck tumors.

Tumor DNA and matched normal DNA samples from each patient were prepared, sequenced, and analyzed as detailed in the working example.

### Example 4: Detection of ctDNA Post-Surgery

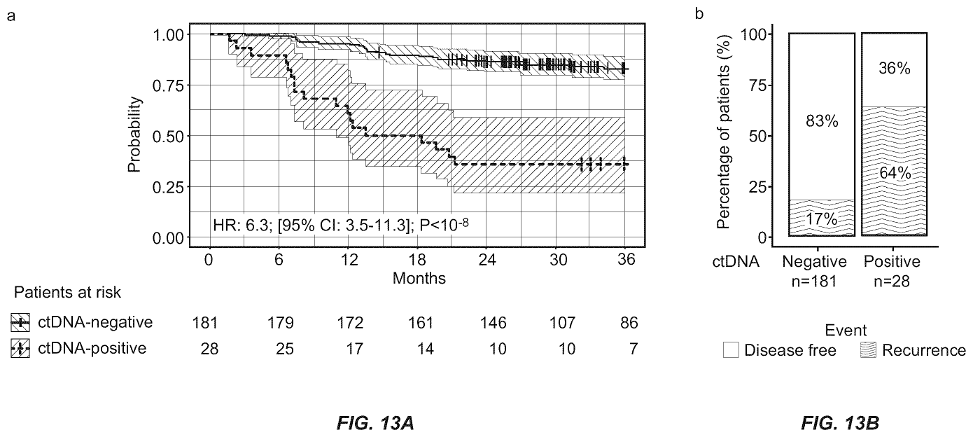

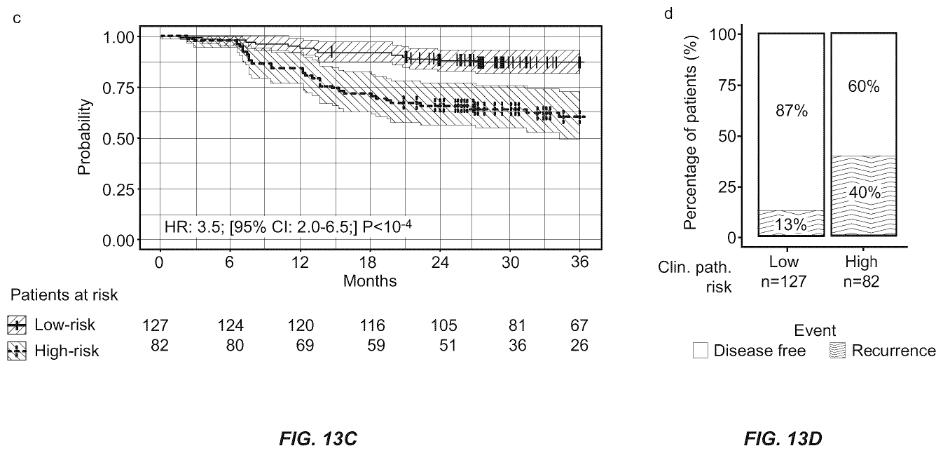

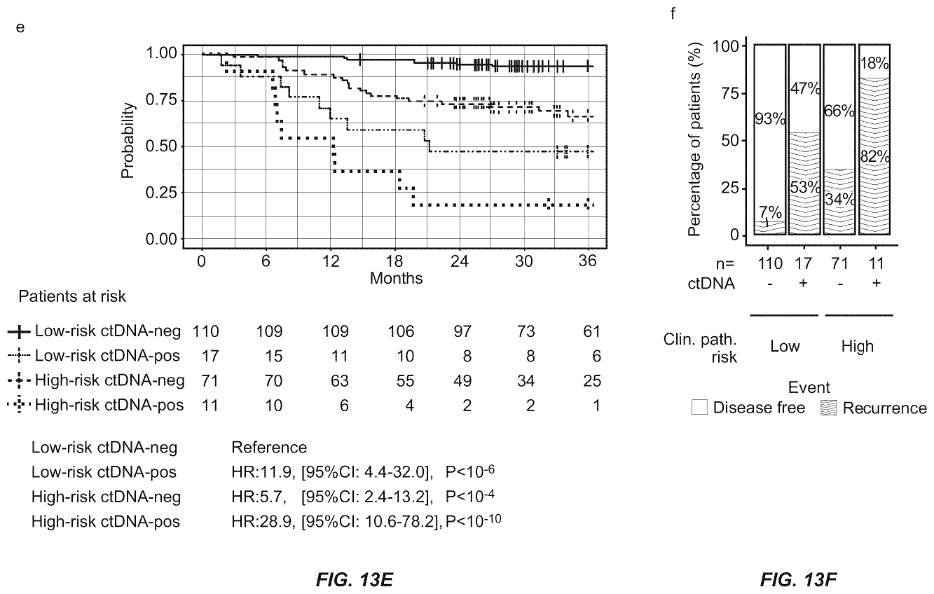

FIG. 13A shows a Kaplan-Meier estimate for time to recurrence (TTR) stratified by post-surgery ctDNA status. Post-surgery ctDNA-positive ACT-treated patients had a higher risk of recurrence compared to patients without detectable ctDNA post-surgery (hazard ratio (FIR) 6.3 [95% confidence interval (CI): 3.5-11.3]; P<10⁻⁸). Median follow-up did not differ between patients who were ctDNA-positive and ctDNA-negative after surgery (P=0.2). FIG. 13B shows the proportion of patients at risk of recurrence after three years. Of the 209 patients assessed, 181 were found to be ctDNA negative while 28 were found to be ctDNA positive. Of the ctDNA negative patients, 83% did not have recurrence and were found disease free after three years. On the other hand, 17% of ctDNA negative patients did show recurrence. Of the ctDNA positive patients, only 36% were disease free, while 64% showed recurrence indicating that patients with a positive ctDNA status post-surgery are at a higher risk of experiencing a recurrence.

Next a prognostic value of ctDNA in the context of an established clinicopathological risk stratification factor for recurrence in stage III colon cancer was assessed. High-risk patients have a risk factor of (pT4pT4 and/or pN2pN2) and low-risk patients have a risk factor of (pT1pT1-3N1), where “T” refers to tumor status and “N” refers to lymph node status. FIG. 13C shows a Kaplan-Meier estimate for TTR stratified by clinicopathological risk (HR 3.5 [95% CI: 2.0-6.5]; P<10⁻⁴. Table 4 shows univariate analyses for post-surgery ctDNA status and clinicopathological risk factors. FIG. 13D shows the proportion of patients at risk of recurrence after three years. 127 patients were found to have low risk of recurrence where 83% of low-risk patients did not experience a recurrence while 13% did. 82 patients were found to have a high-risk of recurrence. Of those, 60% did not experience recurrence while 40% did experience a recurrence of disease.

TABLE 4
Univariate cox regression analyses for Post-surgery
ctDNA status and clinicopathological risk

| | | % | | | |
|---|---|---|---|---|---|
| Risk | | Pa- | | | |
| variable | Groups | tients | HR | 95% CI | p. value |
| Post-surgery | ctDNA-negative | 181 | 6.26 | 3.46-11.31 | 1.24e−09 |
| ctDNA | ctDNA-positive | 28 | | | |
| status | | | | | |
| Clinico- | Low risk | 127 | 3.54 | 1.94-6.49 | 4.05e−05 |
| patholog- | High risk | 82 | | | |
| ical risk | | | | | |
| T status | T1-3 | 158 | 3.00 | 1.69-5.34 | 0.000187 |
| | T4 | 51 | | | |
| N status | N1 | 154 | 3.21 | 1.81-5.69 | 6.72e−05 |
| | N2 | 55 | | | |
| MSS | Stable (MSS) | 179 | 0.38 | 0.12-1.20 | 0.104 |
| status | Unstable (MSI) | 30 | | | |

As shown in FIG. 13E and Table 5, multivariable analysis defined four groups. according to the combination of clinicopathological risk and post-surgery ctDNA status. Recurrence risk of clinicopathological high-risk patients was further increased when patients were ctDNA-positive, while recurrence risk of clinicopathological low-risk patients was further decreased when patients were ctDNA-negative. Consequently, there is a profound survival difference between clinicopathological high risk ctDNA-positive patients and clinicopathological low-risk ctDNA-negative patients (three-year risk of recurrence 82% versus 7%) as shown in FIG. 13F (HR 28.9 [95% CI: 10.6-78.2]; P<10⁻¹⁰).

TABLE 5
Univariate cox regression analyses per individual clinicopathological
risk variable including ctDNA status.

| Risk variable | Groups | ctDNA status | HR | 95% CI | p-value |
|---|---|---|---|---|---|
| Clinicopathological | Low risk | ctDNA- | Reference | | |
| risk + Post- | | negative | | | |
| surgery ctDNA status | | ctDNA- | 11.88 | 4.42-31.95 | 9.34e−07 |
| | | positive | | | |
| | High risk | ctDNA- | 5.65 | 2.41-13.24 | 6.64e−05 |
| | | negative | | | |
| | | ctDNA- | 28.86 | 10.65-78.26 | 3.92e−11 |
| | | positive | | | |
| T status + | T1-3 | ctDNA- | Reference | | |
| Post-surgery | | negative | | | |
| ctDNA status | | ctDNA- | 6.51 | 2.99-14.21 | 2.45e−06 |
| | | positive | | | |
| | T4 | ctDNA- | 3.30 | 1.59-6.83 | 0.00135 |
| | | negative | | | |
| | | ctDNA- | | 15.29-101.36 | |
| | | positive | 39.37 | | 2.68e−14 |
| N status + | N1 | ctDNA- | Reference | | |
| Post-surgery | | negative | | | |
| ctDNA status | | ctDNA- | 8.81 | 3.94-19.70 | 1.14e−07 |
| | | positive | | | |
| | N2 | ctDNA- | 4.19 | 2.02-8.73 | 0.000125 |
| | | negative | | | |
| | | ctDNA- | 17.21 | 6.80-43.57 | 1.94e−09 |
| | | positive | | | |
| MSS status + | Unstable | ctDNA- | Reference | | |
| Post-surgery | (MSI) | negative | | | |
| ctDNA status | | ctDNA- | 4.79 | 0.50-46.11 | 0.17 |
| | | positive | | | |
| | Stable | ctDNA- | 1.63 | 0.50-5.36 | 0.42 |
| | (MSS) | negative | | | |
| | | ctDNA- | 10.05 | 2.94-34.40 | 0.000236 |
| | | positive | | | |

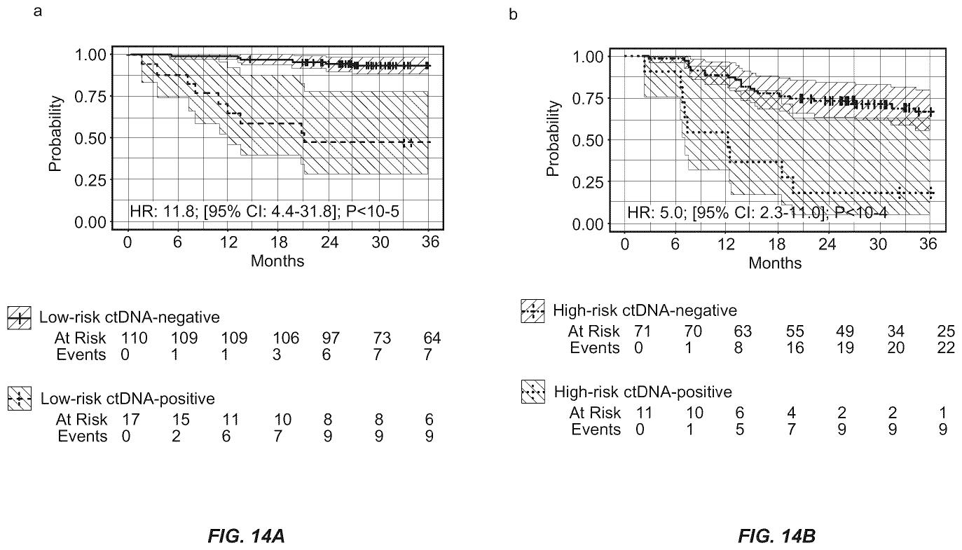

In further support of FIGS. 13E and 13D, FIG. 14 shows Kaplan-Meier estimates for cox regression analyses for clinicopathological low-risk (FIG. 14A) and high-risk (FIG. 14B) groups stratified by post-surgery ctDNA status with confidence intervals. Post-surgery ctDNA status patients with a positive ctDNA status post-surgery are at a higher risk of experiencing a recurrence and are more likely to relapse sooner compared to ctDNA negative patients also at high-risk.

Furthermore, the value of post-surgery ctDNA status added to a multivariable Cox model including clinicopathological risk and MSS status was assessed by performing a likelihood ratio test (LRT) including or excluding ctDNA status. As shown in Table 6, inclusion of ctDNA status significantly improved the model (LRT P<10-7). Multivariate Cox regression models were fitted to include different clinicopathological variables. In addition, four likelihood ratio tests were performed to assess the added value of ctDNA status in each model. It was found that ctDNA status corresponds to the post-surgery timepoint.

TABLE 6
Likelihood Ratio Test for model goodness-of-fit assessment.

| | Predictor combinations in cox | | |
|---|---|---|---|
| | regression model | P-value | Better model |
| LRT | Clinicopathological_Risk | 1.45e−08 | Clinicopathological_Risk + |
| 1 | Clinicopathological_Risk + | | ctDNA status |
| | ctDNA status | | |
| LRT | Clinicopathological_Risk + | 2.23e−08 | Clinicopathological_Risk + |
| 2 | MSS status | | MSI + ctDNA status |
| | Clinicopathological_Risk + | | |
| | MSS + ctDNA status | | |
| LRT | T status + N status | 8.54e−09 | T status + N status + |
| 3 | T status + N status + | | ctDNA status |
| | ctDNA status | | |
| LRT | T status + N status + MSS status | 1.00e−08 | T status + N status + |
| 4 | T status + N status + MSS | | MSI status + ctDNA status |
| | status + ctDNA status | | |

As shown in Table 7, ctDNA status was the strongest independent predictor of recurrence (HR 6.8) in a model that included clinicopathological risk (HR 4.0) and microsatellite stability (MSS) status (HR 0.7, ns).

TABLE 7
Multivariate cox regression model analysis with hazard ratio per variable included.

| | | | Hazard | 95% | p- |
|---|---|---|---|---|---|
| Model | Covariate | Reference | Ratio | CI | value |
| Clinicopathological_Risk + | Clinicopathologcal risk: | Low-risk | 4.02 | 2.20-7.351 | 6.28e−06 |
| MSS status + | High-risk | | | | |
| ctDNA status | MSS status: Stable | Instable | 0.72 | 0.26-2.03 | 0.54 |
| | ctDNA status: ctDNA- | ctDNA- | 6.75 | 3.73-12.22 | 2.75e−10 |
| | positive | negative | | | |
| T status + N status + | T status: T4 | T1-3 | 3.10 | 1.69-5.70 | 0.000263 |
| MSS status + | N status: N2 | N1 | 2.58 | 1.41-4.72 | 0.002033 |
| ctDNA status | MSS status: Stable | Instable | 0.84 | 0.29-2.42 | 0.75 |
| | ctDNA status: ctDNA- | ctDNA- | 7.43 | 4.02-13.74 | 1.6e−10 |
| | positive | negative | | | |

In summary, Tables 4-7 show the effects of tumor (T), node (N) and MSS status as independent risk factors in multivariable models, in which post-surgery ctDNA status remained the strongest predictor of recurrence.

### Time to Recurrence

FIG. 15 shows the estimated time to recurrence based on ctDNA status. FIGS. 16A and 16B show that among the 47 out of 209 (22%) patients who experienced a recurrence, ctDNA-positive patients tended to have a shorter time to recurrence compared to ctDNA-negative patients. Also shown in FIG. 15B is that among the 28 post-surgery ctDNA-positive patients, 7 (25%) remained disease-free for at least 36 months post-surgery, suggesting they may have benefited from ACT treatment. Next, post-ACT ctDNA analysis for 170 out of 209 patients. As shown in FIGS. 16C and 16D, ctDNA-positive patients were at a higher risk of developing a recurrence (HR 7.9 [95% CI: 3.55-15.9]; P<0−8). Further details about post-ACT ctDNA status, ctDNA clearance and recurrence location is provided in Table 2 and Table 4.

### DISCUSSION

Stratification based on clinicopathological risk and post-surgery ctDNA-status can guide shared ACT decisions. De-escalation or withholding of adjuvant treatment in low clinicopathological risk post-surgery ctDNA-negative patients should be evaluated in a clinical trial, together with appropriate MRD surveillance. The clinical sensitivity of ctDNA to detect disease recurrence in the PROVENC3 study indicates that ctDNA is detectable about 6 to 10 months prior to a clinically detected recurrence. This provides opportunities for evaluating interventions in studies designed for this selected patient population.

In conclusion, the PROVENC3 study demonstrates the strong potential of MRD testing by a tumor informed WGS-based plasma ctDNA approach and enables the robust design of clinical practice changing interventional ctDNA-guided studies that improve disease management of patients with stage III colon cancer.

### VIII. ADDITIONAL CONSIDERATIONS

Implementation of the techniques, blocks, steps, and means described above can be done in numerous ways. For example, these techniques, blocks, steps, and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to, portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

Publications cited herein and the material for which they are cited are hereby specifically incorporated by reference.

1. A computer implemented method comprising:
generating sequence reads from a tumor nucleic acid sample, a noncancerous nucleic acid sample, and a non-tissue nucleic acid sample collected from the same patient, wherein the sequence reads are generated using whole genome sequencing (WGS);
generating a tumor variant call file, a noncancerous variant call file, and a non-tissue variant call file by analyzing the sequence reads corresponding respectively to the tumor nucleic acid sample, the noncancerous nucleic acid sample, and the non-tissue sample;
comparing the tumor variant call file to the noncancerous variant call file to generate a list of somatic variants;
comparing the list of somatic variants to the non-tissue variant call file to generate a list of candidate somatic variants;
generating, by a classification machine learning model, scores for each of the candidate somatic variants in the list of candidate somatic variants, wherein the scores are generated based on a plurality of classifications generated by the classification machine learning model;
determining, based on the scores, a ctDNA status for the patient, wherein the ctDNA status is either positive or negative; and
generating a report that provides the ctDNA status for the patient.

2. (canceled)

3. The computer implemented method of claim 1, wherein the tumor nucleic acid sample is cancer positive tissue, wherein the noncancerous nucleic acid sample is white blood cells, and wherein the non-tissue nucleic acid sample is plasma.

4. (canceled)

5. The computer implemented method of claim 3, wherein the noncancerous nucleic acid sample and the non-tissue nucleic acid sample are collected from the same whole blood sample.

6. The computer implemented method of claim 1, wherein the tumor nucleic acid sample is sequenced to a depth of at least 50×, wherein the noncancerous nucleic acid sample is sequenced to a depth of at least 30×, and wherein the non-tissue nucleic acid sample is sequenced to a depth of at least 20×.

7. The computer implemented method of claim 6, wherein the tumor nucleic acid sample is sequenced to a depth of 80×, wherein the noncancerous nucleic acid sample is sequenced to a depth of 40×, and wherein the non-tissue nucleic acid sample is sequenced to a depth of 30×.

8. The computer implemented method of claim 1, wherein the patient is diagnosed with cancer, received surgery to remove one or more tumors, and received a therapeutic treatment post-surgery.

9. (canceled)

10. The computer implemented method of claim 8, wherein the patient is diagnosed with colorectal cancer, head and neck cancer, lung cancer, breast cancer, or melanoma.

11. (canceled)

12. The computer implemented method of claim 1, wherein the tumor nucleic acid sample, the noncancerous samples, and the non-tissue samples are collected (i) pre-surgery, (ii) during surgery, (iii) about 3 days to about 65 days post-surgery and before receiving a therapeutic treatment, (iv) about every 6 months up to 3 years post-surgery and after receiving the therapeutic treatment, or (v) any combination thereof.

13. The computer implemented method of claim 1, wherein the tumor variant call file and the noncancerous variant call file are filtered using a set of filtering criteria, and wherein the set of filtering criteria include removing:(i) variants annotated as low confidence, (ii) variants annotated as indels, (iii) variants observed in genomic databases, (iv) variants overlapping simple tandem repeat tracks, (v) variants at genomic positions with less than 10× coverage, (vi) variants at genomic positions with an alternate allele count less than 4 in the tumor nucleic acid sample or greater than 1 in the noncancerous nucleic acid sample, (vii) variants with a variant allele frequency less than 0.05, or (viii) any combination thereof.

14. The computer implemented method of claim 1, wherein the list of candidate somatic variants comprises substitutions, small indels, chromosomal rearrangements, copy number variation, microsatellite instabilities, or any combination thereof.

15. The computer implemented method of claim 14, wherein the list of candidate somatic variants includes at least 40,000 to at least 70,000 somatic variants.

16. The computer implemented method of claim 15, wherein each candidate somatic variant on the list of candidate somatic variants has at least 50 corresponding features.

17. The computer implemented method of claim 16, wherein the features comprise quality metrics output from sequencing, alignment, and variant calling.

18. (canceled)

19. The computer implemented method of claim 1, wherein prior to generating the scores, the classification model filters, using a set of noncancerous donor samples, the list of candidate somatic variants to generate a filtered list of candidate somatic variants.

20. The computer implemented method of claim 1, wherein the classification machine learning model is a random forest classifier comprising an ensemble of trees having at least 500 decision trees, wherein:
each of the trees generates a score for an input candidate somatic variant,
the random forest classifier averages the scores generated by each of the trees to determine a final score,
the final score is compared to a predetermined threshold to determine whether a ctDNA status of the non-tissue nucleic acid sample is positive or negative,
the ensemble of trees considers at least 50 features associated with the candidate somatic variants, and
each tree considers a different subset of features from the at least 50 features to make a prediction for the class.

21. (canceled)

22. The computer implemented method of claim 20, wherein the final score is greater than or equal to the predetermined threshold and the ctDNA status is positive, and wherein the final score is less than the predetermined threshold and the ctDNA status is negative.

23. (canceled)

24. (canceled)

25. The computer implemented method of claim 1, wherein the ctDNA status is correlated with clinicopathological risk factors to predict survival rate, wherein the clinicopathological risk factors predict recurrence risk, and wherein the clinicopathological risk factors include depth of tumor invasion and spread of tumor to neighboring lymph nodes.

26. The computer implemented method of claim 25, wherein the correlation between the ctDNA status and the clinicopathological risk factors is included in the report, and wherein the report further describes a recurrence risk and a predicted survival rate of the patient, based on the ctDNA status and clinicopathological risk factors of the patient.

27. (canceled)

28. (canceled)

29. A system comprising:
one or more processors; and
one or more computer-readable media storing instructions which, when executed by the one or more processors, cause the system to perform operations comprising:
generating sequence reads from a tumor nucleic acid sample, a noncancerous nucleic acid sample, and a non-tissue nucleic acid sample collected from the same patient, wherein the sequence reads are generated using whole genome sequencing (WGS);
generating a tumor variant call file, a noncancerous variant call file, and a non-tissue variant call file by analyzing the sequence reads corresponding respectively to the tumor nucleic acid sample, the noncancerous nucleic acid sample, and the non-tissue sample;
comparing the tumor variant call file to the noncancerous variant call file to generate a list of somatic variants;
comparing the list of somatic variants to the non-tissue variant call file to generate a list of candidate somatic variants;
generating, by a classification machine learning model, scores for each of the candidate somatic variants in the list of candidate somatic variants, wherein the scores are generated based on a plurality of classifications generated by the classification machine learning model;
determining, based on the scores, a ctDNA status for the patient, wherein the ctDNA status is either positive or negative; and
generating a report that provides the ctDNA status for the patient.

30. One or more non-transitory computer-readable media storing instructions which, when executed by one or more processors, cause a system to perform operations comprising:
generating sequence reads from a tumor nucleic acid sample, a noncancerous nucleic acid sample, and a non-tissue nucleic acid sample collected from the same patient, wherein the sequence reads are generated using whole genome sequencing (WGS);
generating a tumor variant call file, a noncancerous variant call file, and a non-tissue variant call file by analyzing the sequence reads corresponding respectively to the tumor nucleic acid sample, the noncancerous nucleic acid sample, and the non-tissue sample;
comparing the tumor variant call file to the noncancerous variant call file to generate a list of somatic variants;
comparing the list of somatic variants to the non-tissue variant call file to generate a list of candidate somatic variants;
generating, by a classification machine learning model, scores for each of the candidate somatic variants in the list of candidate somatic variants, wherein the scores are generated based on a plurality of classifications generated by the classification machine learning model;
determining, based on the scores, a ctDNA status for the patient, wherein the ctDNA status is either positive or negative; and
generating a report that provides the ctDNA status for the patient.

31. (canceled)

32. (canceled)

33. (canceled)

34. (canceled)
